SEQUENCE LISTING (11 sequences)

SEQ ID NO: 1 - DNA, 30 bp, Artificial Sequence
  Forward primer
gcatgcgccaaggaaaagtttgagcggacc

SEQ ID NO: 2 - DNA, 31 bp, Artificial Sequence
  Reverse primer
aagcttttactcgatgatcttggcgacgacg

SEQ ID NO: 3 - protein, 396 aa, Burkholderia thailandensis E264
MAKEKFERTKPHVNVGTIGHVDHGKTTLTAAIATVLSAKFGGEAKKYDEIDAAPEEKARGITINTAHIEYETANRHYAHVDCPGHADYVKNMITGAAQMDGAILVCSAADGPMPQTREHILLARQVGVPYIIVFLNKCDMVDDAELLELVEMEVRELLSKYDFPGDDTPIIKGSAKLALEGDKGELGEVAIMNLADALDTYIPTPERAVDGAFLMPVEDVFSISGRGTVVTGRVERGVIKVGEEIEIVGIKATAKTTCTGVEMFRKLLDQGQAGDNVGILLRGTKREDVERGQVLAKPGSITPHTHFTAEVYVLSKDEGGRHTPFFNNYRPQFYFRTTDVTGSIELPKDKEMVMPGDNVSITVKLIAPIAMEEGLRFAIREGGRTVGAGVVAKIIE

SEQ ID NO: 4 - protein, 396 aa, Burkholderia pseudomallei K96243; identical to SEQ ID NO: 3

SEQ ID NO: 5 - protein, 396 aa, Burkholderia mallei ATCC 23344; identical to SEQ ID NO: 3

SEQ ID NO: 6 - protein, 394 aa, Escherichia coli str. K-12 substr. MG1655
MSKEKFERTKPHVNVGTIGHVDHGKTTLTAAITTVLAKTYGGAARAFDQIDNAPEEKARGITINTSHVEYDTPTRHYAHVDCPGHADYVKNMITGAAQMDGAILVVAATDGPMPQTREHILLGRQVGVPYIIVFLNKCDMVDDEELLELVEMEVRELLSQYDFPGDDTPIVRGSALKALEGDAEWEAKILELAGFLDSYIPEPERAIDKPFLLPIEDVFSISGRGTVVTGRVERGIIKVGEEVEIVGIKETQKSTCTGVEMFRKLLDEGRAGENVGVLLRGIKREEIERGQVLAKPGTIKPHTKFESEVYILSKDEGGRHTPFFKGYRPQFYFRTTDVTGTIELPEGVEMVMPGDNIKMVVTLIHPIAMDDGLRFAIREGGRTVGAGVVAKVLS

SEQ ID NO: 7 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

SEQ ID NO: 8 - protein, 396 aa, Burkholderia pseudomallei Pasteur 52237; identical to SEQ ID NO: 3

SEQ ID NO: 9 - protein, 396 aa, Burkholderia pseudomallei 406e; identical to SEQ ID NO: 3

SEQ ID NO: 10 - protein, 396 aa, Burkholderia pseudomallei 1106a; identical to SEQ ID NO: 3

SEQ ID NO: 11 - protein, 396 aa, Burkholderia pseudomallei MSHR346; identical to SEQ ID NO: 3

(19) United States
(12) Patent Application Publication
(10) Pub. No.: US 2014/0004178 A1
(43) Pub. Date: Jan. 2, 2014

(54) OMV VACCINE AGAINST BURKHOLDERIA INFECTIONS

(71) Applicant: Lisa A. Morici, New Orleans, LA (US)

(72) Inventor: Lisa A. Morici, New Orleans, LA (US)

(73) Assignee: THE ADMINISTRATORS OF THE TULANE EDUCATIONAL FUND, New Orleans, LA (US)

(21) Appl. No.: 13/979,037

(22) PCT Filed: Jan. 12, 2012
(86) PCT No.: PCT/US2012/021128

§ 371 Date: Sep. 18, 2013

Related U.S. Application Data
(60) Provisional application No. 61/432,040, filed on Jan. 12, 2011.

(51) Int. Cl. A61K 39/02

(52) U.S. Cl. CPC A61K 39/0208
USPC 424/450; 424/234.1; 424/241.1; 424/236.1

(57) ABSTRACT

The present disclosure relates to vaccine compositions and methods of using the vaccine compositions to provide protection against various Gram-negative bacterial infections, including Burkholderia infections.

### STATEMENT REGARDING FEDERALLY SPONSORED RESEARCH OR DEVELOPMENT

This invention was made with government support under grants U54 AI057156 awarded by National Institute of Allergy and Infectious Diseases (NIAID)/NIH. The government has certain rights in the invention.

### BACKGROUND

1. Field

The present disclosure relates to antibacterial vaccines and to the prevention of infection by a bacterial pathogen by immunization, generally, and to vaccines against the genus Burkholderia, in particular.

2. Description of Related Art

Burkholderia is a genus of proteobacteria probably best-known for its pathogenic members: Burkholderia mallei, responsible for glanders, a disease that occurs mostly in horses and related animals; Burkholderia pseudomallei, causative agent of melioidosis; and Burkholderia cepacia, an important pathogen of pulmonary infections in people with cystic fibrosis (CF). The Burkholderia (previously part of Pseudomonas) genus name refers to a group of virtually ubiquitous gram-negative, motile, obligately aerobic rod-shaped bacteria including both animal/human and plant pathogens as well as some environmentally-important species. Due to their antibiotic resistance and the high mortality rate from their associated diseases, Burkholderia mallei and Burkholderia pseudomallei are considered potential biological warfare agents, targeting livestock and humans.

The bacterium Burkholderia pseudomallei (Gram-negative, facultative intracellular bacillus) is the causative agent of melioidosis, a serious emerging disease responsible for significant morbidity and mortality in Southeast Asia and Northern Australia [Cheng A C, Currie B J (2005) Melioidosis: epidemiology, pathophysiology, and management. Clin Microbiol Rev 18: 383-416.]. Natural infection can occur through subcutaneous inoculation, ingestion, or inhalation of the organism. Clinical manifestations are nonspecific and widely variable, and may include acute septicemia, pneumonia, and chronic infection [Wiersinga W J, van der Poll T (2009) Immunity to Burkholderia pseudomallei. Curr Opin Infect Dis 22: 102-108]. Mortality rates associated with severe B. pseudomallei infection approach 50% and can reach 80-95% in patients with septic shock, despite antibiotic treatment [Leelarasamee A (2004) Recent development in melioidosis. Curr Opin Infect Dis 17: 131-136; Peacock S J (2006) Melioidosis. Curr Opin Infect Dis 19: 421-428]. This is partially due to the innate antimicrobial resistance of B. pseudomallei as well as the intracellular niche of the organism [Cheng A C, Currie B J (2005) Melioidosis: epidemiology, pathophysiology, and management. Clin Microbiol Rev 18: 383-416; Jones A L, Beveridge T J, Woods D E (1996) Intracellular survival of Burkholderia pseudomallei. Infect Immun 64: 782-790]. Thus, preventive measures such as active immunization are needed to reduce the morbidity and mortality associated with B. pseudomallei infection.

Previous immunization strategies that utilized heat-killed or live-attenuated B. pseudomallei, lipopolysaccharide (LPS), capsular polysaccharide (CPS), or protein-based (i.e. Type III secretion system (TTSS-3) or outer membrane proteins) subunits conferred variable degrees of protection against systemic challenge, but have proved ineffective or have not been tested against aerosol infection [Harland D N, Chu K, Haque A, Nelson M, Walker N J, et al. (2007) Identification of a LolC homologue in Burkholderia pseudomallei, a novel protective antigen for melioidosis. Infect Immun 75: 4173-4180; Jones S M, Ellis J F, Russell P, Griffin K F, Oyston P C (2002) Passive protection against Burkholderia pseudomallei infection in mice by monoclonal antibodies against capsular polysaccharide, lipopolysaccharide or proteins. J Med Microbiol 51: 1055-1062; Nelson M, Prior J L, Lever M S, Jones H E, Atkins T P, et al. (2004) Evaluation of lipopolysaccharide and capsular polysaccharide as subunit vaccines against experimental melioidosis. J Med Microbiol 53: 1177-1182; Haque A, Chu K, Easton A, Stevens M P, Galyov E E, et al. (2006) A live experimental vaccine against Burkholderia pseudomallei elicits CD4+ T cell-mediated immunity, priming T cells specific for 2 type III secretion system proteins. J Infect Dis 194: 1241-1248; Hara Y, Mohamed R, Nathan S (2009) Immunogenic Burkholderia pseudomallei outer membrane proteins as potential candidate vaccine targets. PLoS One 4: e6496; Druar C, Yu F, Barnes J L, Okinaka R T, Chantratita N, et al. (2008) Evaluating Burkholderia pseudomallei Bip proteins as vaccines and Bip antibodies as detection agents. FEMS Immunol Med Microbiol 52: 78-87; Breitbach K, Kohler J, Steinmetz I (2008) Induction of protective immunity against Burkholderia pseudomallei using attenuated mutants with defects in the intracellular life cycle. Trans R Soc Trop Med Hyg 102 Suppl 1: S89-94; Stevens M P, Haque A, Atkins T, Hill J, Wood M W, et al. (2004) Attenuated virulence and protective efficacy of a Burkholderia pseudomallei bsa type III secretion mutant in murine models of melioidosis. Microbiology 150: 2669-2676]. In addition, vaccine preparations administered parenterally with aluminum hydroxide adjuvant elicit robust antibody and Type 2 immune responses against B. pseudomallei but are insufficient for complete protection [Bondi S K, Goldberg J B (2008) Strategies toward vaccines against Burkholderia mallei and Burkholderia pseudomallei. Expert Rev Vaccines 7: 1357-1365]. Antibody responses alone are often deficient in providing sterile immunity against intracellular bacterial pathogens Newman M (1995) Immunological and Formulation Design Considerations for Subunit Vaccines; Newman M, editor. New York: Plenum Press. 1-42 p]. An ideal vaccine against B. pseudomallei will likely require the induction of a Type 1 cellular-mediated immune (CMI) response for complete efficacy, as suggested from past immunization studies [Haque A, Chu K, Easton A, Stevens M P, Galyov E E, et al. (2006) A live experimental vaccine against Burkholderia pseudomallei elicits CD4+ T cell-mediated immunity, priming T cells specific for 2 type III secretion system proteins. J Infect Dis 194: 1241-1248; Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951]. Furthermore, the nasal associated lymphoid tissue (NALT) may represent a primary site of invasion by B. pseudomallei [Owen S J, Batzloff M, Chehrehasa F, Meedeniya A, Casart Y, et al. (2009) Nasal-Associated Lymphoid Tissue and Olfactory Epithelium as Portals of Entry for Burkholderia pseudomallei in Murine Melioidosis. J Infect Dis 199: 1761-1770]. Vaccine strategies that target the mucosal surface and induce Type 1 responses may therefore provide enhanced protection against aerosol infection with B. pseudomallei.

### BRIEF SUMMARY

The present disclosure relates to vaccine compositions and methods of using the vaccine compositions to provide protection against Gram-negative infections, and particularly against various Burkholderia infections. Vaccine targets were identified by employing an immunoproteomic approach to identify a number of novel immunoreactive proteins in B. thailandensis that have potential for use as subunit vaccines against inhalational B. pseudomallei infection. B. thailandensis shares 94% identity with B. pseudomallei at the amino acid level and has served as a useful surrogate for B. pseudomallei in multiple studies [Stevens J M, Ulrich R L, Taylor L A, Wood M W, Deshazer D, et al. (2005) Actin-binding proteins from Burkholderia mallet and Burkholderia thailandensis can functionally compensate for the actin-based motility defect of a Burkholderia pseudomallei bimA mutant. J Bacteriol 187: 7857-7862; Kim H S, Schell M A, Yu Y, Ulrich R L, Sarria S H, et al. (2005) Bacterial genome adaptation to niches: divergence of the potential virulence genes in three Burkholderia species of different survival strategies. BMC Genomics 6: 174; West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17; Wiersing a W J, de Vos A F, de Beer R, Wieland C W, Roelofs J J, et al. (2008) Inflammation patterns induced by different Burkholderia species in mice. Cell Microbiol 10: 81-87]. The present disclosure provides a novel role for Burkholderia outer membrane vesicles (OMVs, as a vaccine immunogen and demonstrated herein is its ability to elicit antibody responses in immunized mice. Furthermore, the protective capacity of OMV immunization in a B. pseudomallei lethal aerosol challenge model is presented herein.

The present disclosure provides a composition comprising outer membrane vesicles of Gram-negative bacteria, for use as a vaccine. The composition of the present disclosure further comprises lipopolysaccharide, and lacks added adjuvant. The composition of the present disclosure further comprises outer membrane vesicles wherein the vesicles comprise lipopolysaccharide, and lack added adjuvant.

The outer membrane vesicles may be derived from at least one Burkholderia spp. The at least one Burkholderia spp. may be B. ambifaria, B. andropogonis, B. anthina, B. brasilensis, B. caledonica, B. caribensis, B. caryophylli, B. cenocepacia, B. cepacia, B. cepacia complex, B. dolosa, B. fungorum, B. gladioli, B. glathei, B. glumae, B. graminis, B. hospita, B. kururiensis, B. mallei, B. multivorans, B. oklahomensis, B. phenazinium, B. phenoliruptrix, B. phymatum, B. phytofirmans, B. plantarii, B. pseudomallei, B. pyrrocinia, B. sacchari, B. singaporensis, B. sordidicola, B. stabilis, B. terricola, B. thailandensis, B. tropica, B. tuberum, B. ubonensis, B. unamae, B. vietnamiensis, B. xenovorans, or any combinations thereof.

The present disclosure provides a method of protecting a mammal against infection caused by Gram-negative bacteria, the method comprising administering at least one of the aforementioned compositions of outer membrane vesicles. Preferably, the infection is caused by a Burkholderia spp., and the outer membrane vesicles are derived from a Burkholderia spp., preferably the same species.

The present disclosure provides a method of producing a vaccine against Gram-negative bacteria, in particular a vaccine against various Burkholderia, the method comprising:
a. Growing a culture of Gram-negative bacteria;
b. Optionally subjecting the culture to stress during said growth (e.g., via exposure to different temperatures, different pH, nutrient deprivation, antibiotics, and combinations thereof);
c. Pelleting whole bacteria from said culture by centrifugation to obtain a cell pellet and a supernatant fraction;
d. Harvesting outer membrane vesicles from the supernatant; and
e. Further purifying the outer membrane vesicles by density gradient centrifugation.

In an embodiment, optional step (b) comprises subjecting the culture to oxidative stress during growth.

The present disclosure also provides a vaccine produced by the aforementioned method. The compositions may be administered intraperitoneally (IP), intranasally (IN), subcutaneously (SQ), intramuscularly (IM), transdermally, orally, topically, as an aerosol, or via any other commonly known route of administration. The compositions may be provided as an aerosol, a liquid, a suspension, or any other pharmaceutically-acceptable formulation known to those of ordinary skill in the art.

The compositions may be administered in an amount from about 25 ng to about 25 mg, from about 50 ng to about 20 mg, from about 75 ng to about 15 mg, from about 100 ng to about 10 mg, from about 150 ng to about 7.5 mg, from about 0.2 μg to about 5 mg, from about 0.25 μg to about 2.5 mg, from about 0.5 μg to about 2 mg, from about 0.75 μg to about 1.5 mg, from about 1 μg to about 1 mg, from about 1.5 μg to about 750 μg, from about 2 μg to about 500 μg, from about 2.5 μg to about 250 μg, from about 5 μg to about 150 μg, from about 10 μg to about 100 μg, from about 15 μg to about 75 μg, from about 15 μg to about 50 μg, from about 15 μg to about 35 μg, and preferably about 25 μg of OMVs per immunization.

The present disclosure provides methods of protecting a mammal against infection caused by Gram-negative bacteria, the method comprising administering a composition comprising outer membrane vesicles of at least one Gram-negative bacteria. In an embodiment, said Gram-negative bacteria is a Burkholderia species and the outer membrane vesicles are derived from the Burkholderia species.

The present disclosure also provides methods of protecting a subject against infection caused by at least one species of Burkholderia, the method comprising: administering to the subject an immunogenic composition comprising purified outer membrane vesicles derived from at least one species of Burkholderia; wherein administration of the immunogenic composition provides protection against infection. In an embodiment of the present methods, the immunogenic composition is administered subcutaneously, intranasally, and/or intramuscularly. In an embodiment of the present methods, administration of the immunogenic composition produces protective humoral and cellular immunity to at least one species of Burkholderia. In an embodiment of the present methods, the protective humoral immunity in the subject comprises production of IgG and/or IgA specific to the administered outer membrane vesicles. In an embodiment of the present methods, production of IgG specific to the administered outer membrane vesicles increases by at least about 1-log when the immunogenic composition is subsequently administered. In an embodiment of the present methods, the IgG specific to the administered outer membrane vesicles comprises IgG1 and/or IgG2a. In an embodiment of the present methods, the protective cellular immunity in the subject comprises activation of memory T cells in response to the administered outer membrane vesicles. In an embodiment of the present methods, activation of memory T cells comprises production of interferon-gamma (IFN-γ) by Th1 memory cells. In an embodiment of the present methods, administration of the immunogenic composition provides protection when the subject is subsequently exposed to an aerosol challenge comprising at least one species of Burkholderia. In an embodiment of the present methods, the aerosol challenge comprises a lethal dose of the at least one species of Burkholderia. In an embodiment of the present methods, the subject is protected against infection caused by Burkholderia pseudomallei and/or Burkholderia mallei, and wherein the immunogenic composition comprises purified outer membrane vesicles derived from at least Burkholderia pseudomallei and/or Burkholderia mallei.

The present disclosure also provides methods of inducing an immune response to at least one species of Burkholderia in a subject, said method comprising: administering an immunogenic composition comprising at least one purified outer membrane vesicle derived from at least one species of Burkholderia to a subject in an amount effective to elicit production of antibodies specific to the at least one species of Burkholderia. In an embodiment of the present methods, the immunogenic composition is produced by: (a) growing a culture of Gram-negative bacteria; (b) subjecting said culture to centrifugation, thereby obtaining a cell pellet and a supernatant fraction; (c) harvesting outer membrane vesicles from the supernatant fraction; (d) purifying the outer membrane vesicles harvested from step (c) by gradient centrifugation; and (e) collecting the outer membrane vesicles purified from step (d). In an embodiment of the present methods, the gradient centrifugation of step (d) comprises high-speed centrifugation followed by density-gradient centrifugation.

The present disclosure also provides methods of preventing respiratory infection in a subject wherein the respiratory infection is caused by at least one species of Burkholderia, said method comprising: administering to the subject an immunogenic composition comprising purified outer membrane vesicles derived from at least one species of Burkholderia; wherein administration of the immunogenic composition prevents at least one symptom of said respiratory infection. In an embodiment of the present methods, the immunogenic composition is administered subcutaneously, intranasally, and/or intramuscularly. In an embodiment of the present methods, the respiratory infection is caused by Burkholderia pseudomallei and/or Burkholderia mallei, wherein the immunogenic composition comprises purified outer membrane vesicles derived from at least Burkholderia pseudomallei and/or Burkholderia mallei.

The present disclosure also provides methods of preventing meliodosis in a subject wherein the meliodosis is caused by at least one species of Burkholderia, said method comprising: administering to the subject an immunogenic composition comprising purified outer membrane vesicles derived from at least one species of Burkholderia; wherein administration of the immunogenic composition produces immunity to said at least one species of Burkholderia; and wherein administration of the immunogenic composition prevents at least one symptom of said meliodosis. In an embodiment of the present methods, the immunogenic composition is administered subcutaneously, intranasally, and/or intramuscularly. In an embodiment of the present methods, the immunity in the subject is protective humoral and cellular immunity. In an embodiment of the present methods, the protective humoral immunity in the subject comprises production of IgG and/or IgA specific to the administered outer membrane vesicles when the subject is exposed to at least one species of Burkholderia after administration of the immunogenic composition. In an embodiment of the present methods, the protective cellular immunity in the subject comprises activation of memory T cells in response to the administered outer membrane vesicles. In an embodiment of the present methods, activation of memory T cells comprises production of interferon-gamma (IFN-γ) by CD4+ and/or CD8+ T cells. In an embodiment of the present methods, administration of the immunogenic composition provides protection when the subject is subsequently exposed to an aerosol challenge comprising the at least one species of Burkholderia. In an embodiment of the present methods, the meliodosis is pneumonic meliodosis and/or septicemic meliodosis. In an embodiment of the present methods, the meliodosis is caused by Burkholderia pseudomallei, wherein the immunogenic composition comprises purified outer membrane vesicles derived from at least Burkholderia pseudomallei. In an embodiment of the present methods, the immunogenic composition further comprises at least one adjuvant. In an embodiment of the present methods, the at least one adjuvant is selected from the group consisting of methylated CpG oligodeoxynucleotides (CpG ODN), aluminum hydroxide (alum), MPL-monophosphate lipid A, flagellin, cytokines, and toxins. In an embodiment of the present methods, the toxin is E. coli heat-labile enterotoxin and/or cholera toxin. In an embodiment of the present methods, the at least one adjuvant is an emulsions.

The present disclosure also provides methods of preventing respiratory infection in a subject wherein the respiratory infection is caused by at least one species of Burkholderia cepacia complex, said method comprising administering to the subject an immunogenic composition comprising purified outer membrane vesicles derived from the at least one species of Burkholderia cepacia complex; wherein administration of the immunogenic composition produces immunity to said at least one species of Burkholderia cepacia complex; and wherein administration of the immunogenic composition prevents at least one symptom of said respiratory infection. In an embodiment of the present methods, the immunity in the subject is protective humoral and/or cellular immunity. In an embodiment of the present methods, the respiratory infection is rapidly fatal pulmonary infection. In an embodiment of the present methods, the subject is afflicted with cystic fibrosis. In an embodiment of the present methods, the respiratory infection is caused by Burkholderia cenocepacia and/or Burkholderia multivorans, wherein the immunogenic composition comprises purified outer membrane vesicles derived from Burkholderia cenocepacia and/or Burkholderia multivorans.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 depicts B. thailandensis whole cell lysate separated by two-dimensional gel electrophoresis.

FIG. 2 depicts immunogenicity of EF-Tu during infection and immunization using protein detection methods.

FIG. 3 presents data showing EF-Tu is present in B. pseudomallei outer membrane vesicles.

FIG. 4 depicts data showing EF-Tu-specific IgG and IgA concentrations in sera and BAL from immunized mice.

FIG. 5 depicts Th1 and Th2 cytokine responses to rEF-Tu in restimulated splenocytes from immunized mice.

FIG. 6 depicts data of bacterial burden in lungs of EF-Tu immunized and challenged mice.

FIG. 7 depicts B. pseudomallei OMV-specific serum IgG in immunized mice.

FIG. 8 presents Western blot data showing no cross-reactivity of EF-Tu-specific antibody with mammalian tissue.

FIG. 9 presents data showing that EF-Tu is not capable of providing full protection against infection in immunized mice.

FIG. 10 presents data showing that B. pseudomallei OMV provide significant protection against infection in immunized mice.

FIG. 11 presents EF-Tu protein alignment of B. thailandensis E264 (SEQ ID NO:3), B. pseudomallei K96243 (SEQ ID NO:4), B. mallei ATCC 23344 (SEQ ID NO:5), E. coli str. K-12 substr. MG1655 (SEQ ID NO:6), and Homo sapiens (SEQ ID NO:7).

FIG. 12 presents EF-Tu protein alignment of different strains of B. pseudomallei: (B. pseudomallei K96243, which is SEQ ID NO:4; B. pseudomallei Pasteur 52237, which is SEQ ID NO:8; B. pseudomallei 406e, which is SEQ ID NO:9; B. pseudomallei 1106a, which is SEQ ID NO:10; and B. pseudomallei MSHR346, which is SEQ ID NO:11).

FIG. 13 presents characterization of B. pseudomallei OMVs. (13A) Cryo-transmission electron micrograph of B. pseudomallei OMVs. Purified OMVs (0.8 mg/ml) were diluted 1:10 in filtered sterile water for imaging. Image was taken using a JEOL 2010 Transmission Electron Microscope. Bar indicates 100 nm. (B) Western blot demonstrating the presence of capsular polysaccharide (CPS) in B. pseudomallei OMVs. Ten μg of two separate vaccine batches of Bp OMVs (1 and 2) were probed with monoclonal antibody 3C5 specific for B. pseudomallei CPS [33]. B. thailandensis (Bth), which lacks capsule, and B. pseudomallei 1026b (Bp) whole-cell lysates were used as negative and positive controls, respectively.

FIG. 14 presents OMVs shed by broth-grown B. pseudomallei contain immunoreactive antigens. 14(A) SDS-PAGE and Coomassie stain of 5 mg purified OMVs. (14B) OMVs probed with pre-immune serum from a rhesus macaque or (14C) with convalescent serum obtained from the macaque 6 weeks post-infection with 1×106 cfu B. pseudomallei 1026b (1:100 dilution; 2oantibody=goat anti-monkey IgG—HRP conjugated, 1:1000 dilution). MW=molecular weight protein ladder

FIG. 15 presents serum IgG responses to B. pseudomallei OMVs are specific and do not require exogenous adjuvant. Mean reciprocal endpoint titers for B. pseudomallei OMV specific serum IgG are shown for pre-immune sera, and sera obtained 3 weeks after two (1st boost) and three (2nd boost) administrations of 2.5 μg of B. pseudomallei or E. coli OMVs without exogenous adjuvant. Treatment groups (n=5 mice per group) are naïve=non-treated; Ec IN=E. coli OMV-immunized intranasally; B. pseudomallei IN=B. pseudomallei OMV-immunized intranasally; and B. pseudomallei SC=B. pseudomallei OMV-immunized subcutaneously. Asterisks indicate statistical difference of final endpoint titers compared to pre-immune titers within groups (*P<0.05,***P<0.001 using a two-way ANOVA with Bonferroni's post-test).

FIG. 16 presents antibodies directed against multiple proteins are induced by OMV immunization. B. pseudomallei OMVs were probed with pooled sera obtained from (16A) naïve and (16B) OMV SC-immunized mice (n=5 per group) (1:100 dilution; 2o antibody=goat anti-mouse IgG—HRP conjugated, 1:1000 dilution). MW=molecular weight protein ladder

FIG. 17 presents SC immunization with B. pseudomallei OMVs protects mice against lethal aerosol challenge. Mice (n=15 per group) were challenged with 5 LD₅₀ of B. pseudomallei 1026b by small particle aerosol. Composite survival data from two independent experiments is shown through day 14. Mice immunized SC with B. pseudomallei OMVs were significantly protected (P<0.001 using a log-rank Mantel-Cox survival analysis).

FIG. 18 presents B. pseudomallei OMV immunization induces humoral immunity. B. pseudomallei OMV-specific serum IgG (A) and IgA (B) and E. coli OMV specific serum IgG (C) and IgA (D) were measured by ELISA. Microtiter plates were coated with 500 ng/well of purified B. pseudomallei OMVs or E. coli OMVs. Naïve=non-treated; Ec IN=E. coli OMV immunized intranasally; Bp IN=B. pseudomallei OMV-immunized intranasally; and Bp SC=B. pseudomallei OMV-immunized subcutaneously. Horizontal line represents the median value for each group (n=5) (*P<0.05, **P<0.01, ***P<0.001 using a one-way ANOVA with Bonferroni's post-test).

FIG. 19 presents B. pseudomallei OMV immunization induces T cell memory responses. (19A) Splenocytes from individual mice in each group (n=3) were restimulated in triplicate with B. pseudomallei OMVs (2 μg) or ConA (1 μg, not shown) or left unstimulated, and cell culture supernatants were assayed in duplicate on day 3 for IFN-γ cytokine production (**P<0.01, ***P<0.001 using a two-way ANOVA with Bonferroni's post-test).

FIG. 20 presents an exemplary procedure for preparing Burkholderia OMV according to the invention.

FIG. 21 is an illustration of the exemplary OMV immunization strategy employed and described in Example 9.

FIG. 22 demonstrates that mice immunized s.c. with Bps OMVs were significantly protected from pneumonic and septicemic melioidosis. Mice that were immunized with 2.5 μg OMVs s.c., but not i.n., were significantly protected from aerosol challenge. Mice that were immunized s.c. with 5 μg OMVs were significantly protected from i.p. challenge and protection was enhanced by the addition of CpG adjuvant. ** p<0.01; *** p<0.001.

FIG. 23 demonstrates that mice immunized s.c. with Bps OMVs produced significantly higher concentrations of LPS- and CPS-specific serum IgG. Microtiter plates were coated with purified Bth LPS (A) or Bps CPS (B) and serum IgG was measured by ELISA. ** p<0.01; ***p<0.001

FIG. 24 demonstrates that IFN-γ-producing CD8+ T cells are significantly increased in mice immunized s.c. with Bps OMVs. Purified, splenic CD4+ T cells (A) and CD8+ T cells (B) were re-stimulated with Bps OMVs and the frequency of IFN-γ producing cells was enumerated by ELIspot. *** p<0.001

FIG. 25 provides confirmation by Western blot the presence of cross-reactive antigens in Bm and Bps using sera from mice immunized with Bps OMVs.

FIG. 26 illustrates a representative OMV immunization strategy against Bcc, as described in Example 10 herein.

FIG. 27 demonstrates that CpG adjuvant improved OMV vaccine-mediated protection against Bps. Mice (n=10 per group) were challenged with 5 LD₅₀ of Bps K96243 by IP injection. Mice immunized with 5 μg of OMVs (derived from strain 1026b) or 5 μg OMVs admixed with 10 μg CpG ODN were significantly protected compared to control mice (mice that received CpG only or naïve mice) (***P<0.001; **P<0.01 using a log rank Mantel-Cox survival analysis). Note: Two mice in the OMV/CpG group were euthanized due to abscess formation at the site of injection and did not succumb to infection.

FIG. 28 demonstrates that SC immunization with OMVs induced memory CD4+ and CD8+ T cells. Purified, splenic CD4+ and CD8+ T cells from immunized mice (n=5 per group) were re-stimulated with OMVs and the number of IFN-γ producing cells were enumerated by ELIspot. Unstimulated cells and PMA/ionomycin-stimulated cells were used as negative and positive controls respectively. *** P<0.001 using a one-way ANOVA.

FIG. 29 illustrates an exemplary experimental design to evaluate B. pseudomallei OMV vaccine efficacy in non-human primates, as described at Example 11 herein.

FIG. 30 illustrates primates exposed by aerosol to B. pseudomallei 1026b at three target doses (A), with significant bacteria in the blood by +1d PI (B), and in BAL (C) at +1d and +7d PI. Lungs showed signs of hemorrhage from an animal succumbing to disease at +7d PI (D). Animal exposed to approximately <1 log in challenge dose shows less trauma to lung (E). Histopathological analysis indicates focal tracheal necrosis (F), lymphoid hyperplasia (G), and focal inflammation in the liver (H).

FIG. 31 illustrates SDS-PAGE analysis of 2.5 micrograms of B. pseudomallei OMV purified according to exemplary Example 12. Leftmost lane in panels (A)-(F) is a molecular weight protein ladder in which the six predominant blue bands indicate the following molecular weights: 1-250 kilodaltons (kD), 2-150 kD, 3-100 kD, 4-50 kD, 5-20 kD, 6-15 kD. Lanes to the right containing purple bands are the purified OMVs. Panels (A)-(F) refers to varying batches of B. pseudomallei OMV purified according to exemplary Example 12.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

Other than in the operating examples, or where otherwise indicated, all numbers expressing quantities of ingredients, reaction conditions and so forth used in the specification and claims are to be understood as being modified in all instances by the term “about”. Accordingly, unless indicated to the contrary, the numerical parameters set forth in the instant specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the instant disclosure are approximations, the numerical values set forth in the specific examples are reported as precisely as possible. Any numerical values, however, inherently contain certain errors necessarily resulting from the standard deviation found in their respective testing measurements.

It should also be understood that any numerical range recited herein is intended to include all sub-ranges subsumed therein. For example, a range of 1″ to 10″ is intended to include all sub-ranges between and including the recited minimum value of 1 and the recited maximum value of 10, that is, having a minimum value equal to or greater than 1 and a maximum value of equal to or less than 10.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

With respect to the present disclosure, the phrase “effective amount” as used herein is intended to refer to an amount of composition according to the instant disclosure which is sufficient to confer protection against Gram-negative bacterial infection, particularly Burkholderia infection.

Such an amount can vary within a wide range depending on the Gram-negative bacterial organism to be controlled, the immune status of the animal being immunized, the route by which the immunizing composition is administered, and the compounds included in the composition according to the instant disclosure.

With respect to the present disclosure, the phrase “immunogenic composition” as used herein is intended to refer to compositions that elicit, result in, activate an immune response. For example, immunogenic compositions presented herein can eliciting antibodies against at least one species of Burkholderia. In a preferred embodiment, immunogenic compositions presented herein comprise at least one purified outer membrane vesicle derived from at least one species of Burkholderia. With respect to the present disclosure, the term “derived from” as used herein is intended to refer to substances, components, and/or compositions that originate (in whole or in part), grown in, isolated from, and/or comprise substantially similar characteristics with a particular recited organism. For example, immunogenic compositions presented herein comprise at least one purified outer membrane vesicle derived from at least one species of Burkholderia.

The composition of the present disclosure comprises a composition of outer membrane vesicles of Burkholderia, for use as a vaccine. The instant composition further comprises lipopolysaccharide, and lacks adjuvant.

The present disclosure comprises a method of protecting a mammal against infection caused by Burkholderia, the method comprising administering a composition of outer membrane vesicles of Burkholderia.

The composition of the present disclosure comprises a composition for use as a vaccine, produced by the process of a) growing a culture of Gram-negative bacteria; b) optionally subjecting the culture to stress during said growth; c) pelleting whole bacteria from said culture by centrifugation to obtain a cell pellet and a supernatant fraction; d) harvesting outer membrane vesicles from the supernatant; and e) further purifying the outer membrane vesicles by gradient centrifugation. In an embodiment, compositions of the present disclosure are produced by processes wherein step (b) comprises optionally subjecting the culture to oxidative stress during growth.

In one embodiment of the present disclosure, the composition for use as a vaccine, produced by the process of a) Growing a culture of Gram-negative bacteria; further comprises subjecting growing culture of Gram-negative bacteria to oxidative stress during growth, and wherein said oxidative stress comprises ionizing, UV irradiation, oxygen deprivation, and/or chemical agents that generate intracellular oxygen. In an embodiment, compositions of the present disclosure are produced by processes wherein step (b) comprises optionally subjecting the culture to oxidative stress during growth.

Environmental agents such as ionizing, near-UV radiation, or numerous compounds that generate intracellular O2—(redox-cycling agents such as menadione and paraquat) can cause oxidative stress, which arises when the concentration of active oxygen increases to a level that exceeds the cell's defense capacity. Other sources of stress include exposure to temperature, (e.g., 20° C., 25° C., 30° C., 35° C., 40° C., etc., and combinations thereof over time), pH (e.g., about 5 to about 9, about 5.5 to about 8.5, about 6 to about 8, about 6.5 to about 7.5, about 5 to about 6, about 5.5 to about 6.5, about 8 to about 9, and about 7.5 to about 8.5), nutrient deprivation (e.g., limitation of carbon, nitrogen, sulfur, magnesium, vitamins (including B vitamins), etc. and combinations thereof), exposure to antibiotics (e.g., ampicillin, kanamycin, spectinomycin, streptomycin, hygromycin, etc., and combinations thereof), and combinations thereof.

### Vaccine Composition

Vaccines can be developed in different ways, for example by using live bacteria or viruses that have been altered so that they cannot cause disease, killed bacteria or inactivated viruses, toxoids (bacterial toxins that have been made harmless), or purified parts of bacteria or viruses. Vaccines usually contain sterile water or saline, as well as the dead or weakened germ, and other purified components that are included in vaccines because they stimulate the immune system (e.g., adjuvants). Some vaccines are prepared with a preservative or antibiotic (e.g., to prevent bacterial and fungal growth). Some vaccines also are prepared with substances known as stabilizers (e.g., to help the vaccine maintain its effectiveness during storage). Another component of some vaccines is an adjuvant, such as aluminum (to help stimulate the production of antibodies against the vaccine ingredients to make it more effective).

A “vaccine” as referred to herein is defined as a pharmaceutical or therapeutic composition used to inoculate an animal in order to immunize the animal against infection by an organism, typically a pathogenic organism. A vaccine will typically comprise one or more antigens derived from one or more organisms which on administration to an animal will stimulate active immunity and protect that animal against infection with these or related pathogenic organisms. In an embodiment of the invention, immunogenic compositions presented herein comprise adjuvant emulsions. The term “emulsion” as used in the context of the phrase “adjuvant emulsion” herein is intended to refer to emulsion-type adjuvants. Exemplary use of adjuvant emulsions is for optimizing vaccine adjuvant formulation. Emulsion-type adjuvants exhibit various dispersion properties, such as with oil-in-water or water-in-oil types, and can be prepared using emulsifiers with various hydrophilic-hydrophobic balance (HLB) values. The physicochemical properties of the emulsions, including the conductivity and viscosity, and antigen release rates can readily be evaluated to determine immunogenicity-enhancing effect of various well known emulsion adjuvants. See, for example, Yang, Ya-Wun et al., Vaccine, 23(20): 2665-2675 (April 2005), the disclosure of which is incorporated herein by reference. Vaccine compositions that are formulated as pharmaceuticals will typically comprise a carrier. If in solution or in liquid aerosol suspension, suitable carriers can include saline solution, sucrose solution, or other pharmaceutically acceptable buffer solutions. An aerosol formulation will typically additionally comprise a surfactant.

There is currently no effective vaccine against B. pseudomallei, and traditional vaccine attempts have been largely ineffective at preventing the inhalational form of disease in animal models. Therefore, alternative vaccination strategies that incorporate recent advances in adjuvant biology and mucosal immunology deserve investigation. The current approach employs outer membrane vesicles to achieve vaccine protection.

With respect to the present disclosure, the term “prime” as used herein is intended to refer to the first administration of the present immunogenic compositions to a subject. The phrase “single boost” as used herein is intended to refer to the second administration of the present immunogenic compositions to a subject. The single boost is administered after the prime administration. The phrase “second boost” as used herein is intended to refer to the third administration of the present immunogenic compositions to a subject. The second boost is administered after the single boost, which is after the prime administration. It is well understood that the period of time after the prime administration when the single boost and/or second boost is delivered to the subject can vary on the age, health status, and immune status of the subject as well as the particular species of Burkholderia from which the purified OMV's are derived from.

In an embodiment of the invention, the present immunogenic compositions are administered as a prime to a subject. In that embodiment, prime administration of the present immunogenic compositions is sufficient to confer protection of the subject against infection caused by at least one species of Burkholderia.

In another embodiment of the invention, a single boost of the present immunogenic compositions is administered to a subject. In that embodiment, the single boost of the present immunogenic compositions is sufficient to confer protection of the subject against infection caused by at least one species of Burkholderia.

In yet another embodiment of the invention, a second boost of the present immunogenic compositions is administered to a subject. In that embodiment, the second boost of the present immunogenic compositions is sufficient to confer protection of the subject against infection caused by at least one species of Burkholderia.

In yet another embodiment of the invention, a third boost of the present immunogenic compositions is administered to a subject. In that embodiment, the third boost of the present immunogenic compositions is sufficient to confer protection of the subject against infection caused by at least one species of Burkholderia.

Immunoproteomics methods allowed identification of proteins that could be utilized as subunit vaccine antigens and delivered mucosally. Of the 11 proteins identified, three (EF-Tu, AhpC, and DnaK) were previously recognized by Harding et al. [Harding S V, Sarkar-Tyson M, Smither S J, Atkins T P, Oyston P C, et al. (2007) The identification of surface proteins of Burkholderia pseudomallei. Vaccine 25: 2664-2672] using a similar approach with convalescent sera from melioidosis patients. The co-recognition of these particular B. pseudomallei antigens by two independent laboratories reinforces their potential value as vaccine immunogens. One of the three, EF-Tu, was thus selected as the first test antigen since both AhpC and DnaK have received considerable attention elsewhere for related bio-threat agents.

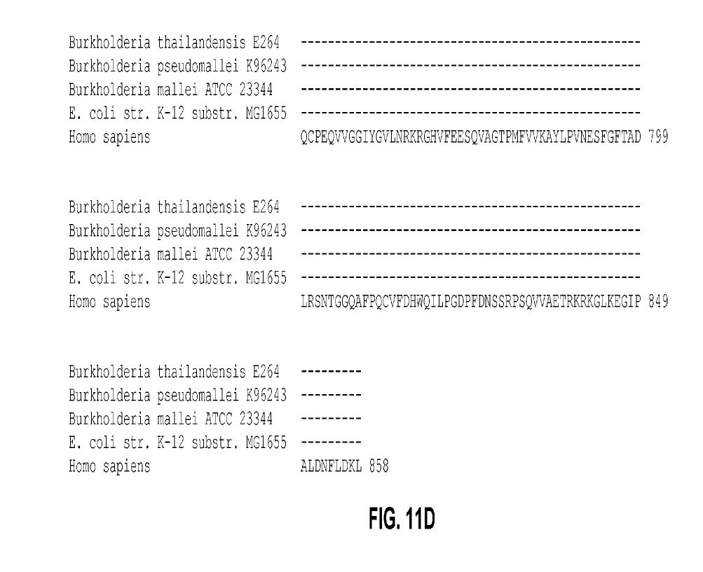

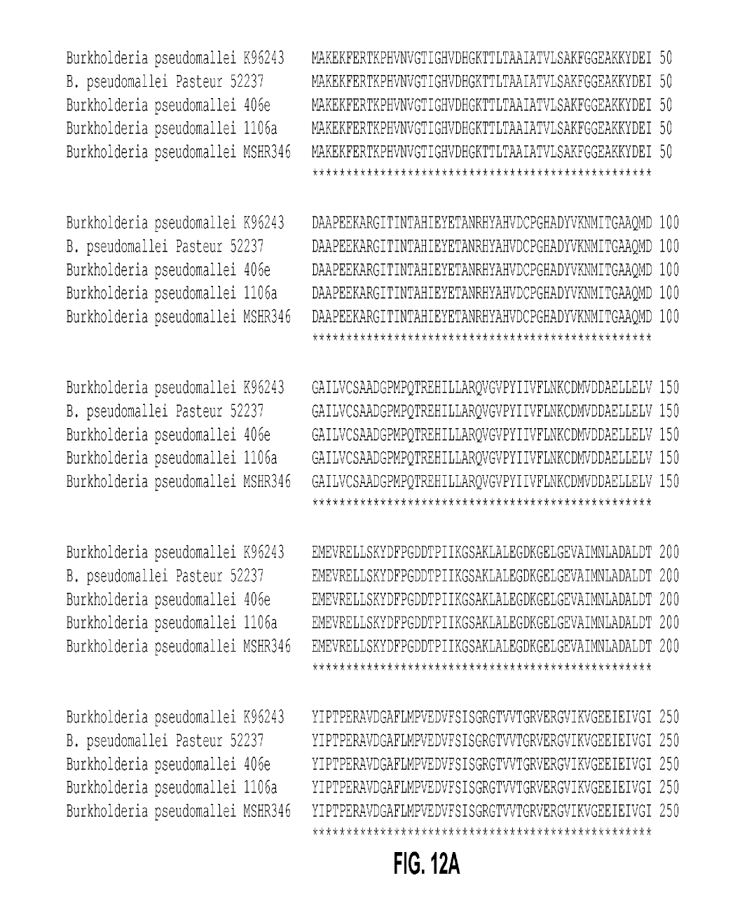

The traditional cytoplasmic role of EF-Tu in protein synthesis would render it an unlikely candidate for a protective subunit vaccine. However, EF-Tu is one of the most abundant and conserved bacterial proteins (100% amino acid identity among B. thailandensis, B. mallei, and five different strains of B. pseudomallei—Table 1, FIG. 11, and FIG. 12—and is a major component of the bacterial membrane cytoskeleton. EF-Tu comprises as much as 5-10% of the cytoplasmic protein in all bacteria investigated, and it may be functionally analogous to actin as it can polymerize into bundle filaments and bind DNase1. Emerging evidence demonstrates that EF-Tu may play a previously under-appreciated role as a bacterial virulence factor. For example, surface-translocated EF-Tu mediates binding to fibronectin and other host proteins for Mycoplasma pneumoniae and Pseudomonas aeruginosa, and EF-Tu can facilitate invasion of host cells by Francisella tularensis via interaction with nucleolin. Furthermore, immunoproteomic-based approaches for antigen discovery against other intracellular bacterial pathogens have identified EF-Tu as an immunodominant protein. Taken together, these studies lend support to the instant observations of immunogenic EF-Tu in the membrane of B. thailandensis, and that reported elsewhere for B. pseudomallei.

### Outer Membrane Vesicles (OMV)

Gram-negative bacteria produce outer membrane vesicles (OMVs) that contain biologically active proteins and perform diverse biological processes. Unlike other secretion mechanisms, OMVs enable bacteria to secrete insoluble molecules in addition to and in complex with soluble material. OMVs allow enzymes to reach distant targets in a concentrated, protected, and targeted form. OMVs also play roles in bacterial survival: Their production is a bacterial stress response and important for nutrient acquisition, biofilm development, and pathogenesis. Key characteristics of OMV biogenesis include outward bulging of areas lacking membrane-peptidoglycan bonds, the capacity to upregulate vesicle production without also losing outer membrane integrity, enrichment or exclusion of certain proteins and lipids, and membrane fission without direct energy from ATP/GTP hydrolysis.

Release of outer membrane (OM) vesicles has been observed for all gram-negative bacteria studied to date. Native vesicles are rounded structures with luminal, periplasmic components bounded by an outer layer of outer membrane proteins (Omps) and lipids. Electron microscopy studies reveal bulging of the OM and subsequent fission of vesicles containing electron-dense material. These biochemical and microscopic observations suggest that OM vesicles are formed from protrusions that are pinched off from the OM in a manner that leads to the inclusion of periplasmic material. The wide variety of strains and diversity of environments for which vesiculation has been observed suggest an important role for vesicle production in gram-negative bacterial growth and survival. Vesicle production varies with growth phase and nutrient availability, and vesicle-associated enzymes may aid in nutrient scavenging. Vesicle-mediated transfer of toxic components to other bacteria can eliminate competing species. In addition, interactions between eukaryotic cells and vesicles from pathogenic bacteria suggest a role for vesicles in pathogenesis. (Journal of Bacteriology, August 2006, p. 5385-5392, Vol. 188, No. 15). These interactions also suggest that OMVs could be useful as immunogenic agents and could confer resistance to bacterial infections.

Although the applicant was not able to demonstrate EF-Tu on the surface of B. thailandensis, EF-Tu was observed in the OMVs shed from B. pseudomallei during in vitro growth. This may partially account for the generation of host antibody against EF-Tu since OMVs have been observed to activate B cells [Amano A, Takeuchi H, Furuta N Outer membrane vesicles function as offensive weapons in host-parasite interactions. Microbes Infect.]

The present disclosure provides OMV purified from Burkholderia and discloses their use in providing immunological protection against Burkholderia infections in mammals.

The present disclosure provides a method of OMV purification, the method comprising growing a culture of Gram-negative bacteria; optionally subjecting the culture to oxidative stress during said growth; pelleting whole bacteria from said culture by centrifugation to obtain a cell pellet and a supernatant fraction; harvesting outer membrane vesicles from the supernatant; and further purifying the outer membrane vesicles by gradient centrifugation.

### Use of Vaccine

The present disclosure provides a method of protecting a mammal against infection caused by Burkholderia, the method comprising administering a vaccine composition comprising outer membrane vesicles (OMVs) of Burkholderia.

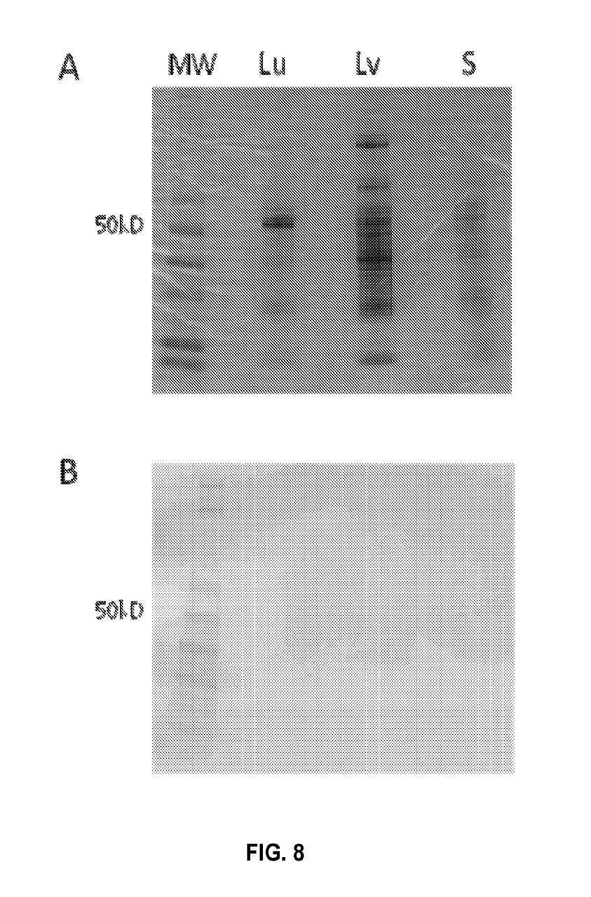

Active immunization of mice with EF-Tu generated high concentrations of antigen-specific IgG that recognized both the recombinant and native forms of EF-Tu. This work represents the first application and evaluation of EF-Tu as a vaccine immunogen for a bacterial pathogen. Like the bacterial antigens flagellin and LPS (both highly-evaluated as vaccine constituents), EF-Tu is abundantly present and highly immunogenic during B. pseudomallei infection in humans Harding S V, Sarkar-Tyson M, Smither S J, Atkins T P, Oyston P C, et al. (2007) The identification of surface proteins of Burkholderia pseudomallei. Vaccine 25: 2664-2672.] and animal models of melioidosis, and thus deserves investigation. Furthermore, bacterial EF-Tu and human EF2 share only 17% identity at the amino acid level and are not functionally interchangeable (FIG. 11A-11D) [Jonak J (2007) Bacterial elongation factors EF-Tu, their mutants, chimeric forms, and domains: isolation and purification. J Chromatogr B Analyt Technol Biomed Life Sci 849: 141-153]. No cross-reactivity of EF-Tu-specific antibody with mammalian tissue was observed by Western blot (FIG. 8). Thus, the potential for bacterial EF-Tu to induce autoimmune disease in vaccinated individuals appears negligible.

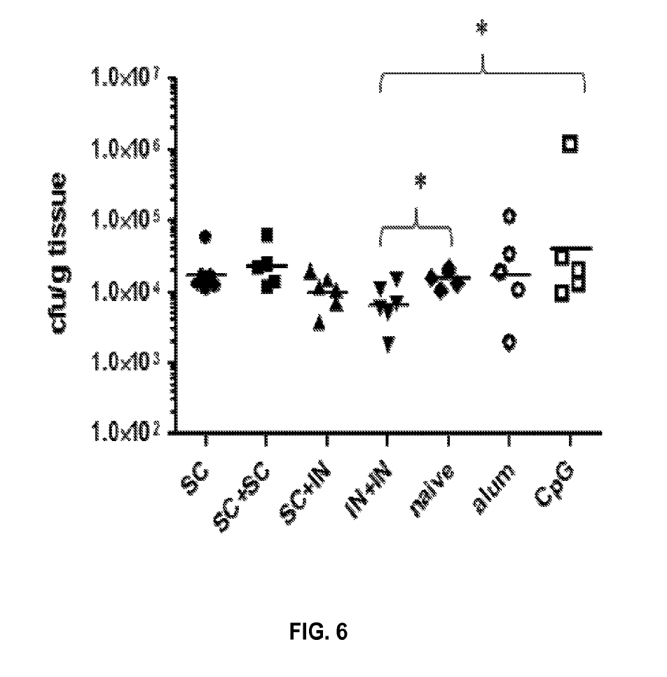

The heterologous and homologous prime/boost immunization studies compared the traditional parenteral route of immunization with aluminum hydroxide as the adjuvant to an intranasal formulation of rEF-Tu admixed with CpG oligodeoxynucleotides (CpG ODN), an adjuvant capable of polarizing the immune response to T-helper 1 cells (Th1) and enhancing mucosal IgA, systemic antibody, and T cell immunity [Freytag L C, Clements J D (2005) Mucosal adjuvants. Vaccine 23: 1804-1813; Klinman D M, Klaschik S, Sato T, Tross D (2009) CpG oligonucleotides as adjuvants for vaccines targeting infectious diseases. Adv Drug Deliv Rev 61: 248-255]. It has been proposed that B. pseudomallei may utilize the nasal-associated lymphoid tissue (NALT) as a portal of entry in murine melioidosis [Owen S J, Batzloff M, Chehrehasa F, Meedeniya A, Casart Y, et al. (2009) Nasal-Associated Lymphoid Tissue and Olfactory Epithelium as Portals of Entry for Burkholderia pseudomallei in Murine Melioidosis. J Infect Dis 199: 1761-1770]. Therefore, the intranasal (i.n.) route of immunization may better prevent mucosal infections through the priming and activation of local antimicrobial immunity. To test this hypothesis, both parenterally and mucosally immunized mice were challenged with 5×105 cfu of B. thailandensis using a nose-only inhalation exposure chamber. Aerosol infection of BALB/c mice with B. thailandensis has previously been demonstrated to be an excellent surrogate model for the acute pneumonic form of disease caused by B. pseudomallei and is capable of reproducing the major lung pathology of murine melioidosis West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17]. Furthermore, there is a direct correlation between lung bacterial burden and disease progression in the murine model [West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17]. The reduced bacterial numbers observed only in the lungs of mice that were immunized mucosally with EF-Tu/CpG suggests that EF-Tu immunization may influence protection and that the route of immunization may be critical. Moreover, the early reduction in bacterial burden in the i.n.+i.n. group cannot exclusively be attributed to the immunoprotective capacity of CpG [Wongratanacheewin S, Kespichayawattana W, Intachote P, Pichyangkul S, Sermswan R W, et al. (2004) Immunostimulatory CpG oligodeoxynucleotide confers protection in a murine model of infection with Burkholderia pseudomallei. Infect Immun 72: 4494-4502] because mice immunized i.n. with CpG ODN 1826 alone had similar, if not slightly higher, numbers of bacteria compared to naïve mice that were challenged (FIG. 6).

Past vaccine attempts against B. pseudomallei failed to confer complete protection despite the induction of a robust antibody response; however, humoral immunity will likely be an essential component of any vaccine against this organism [Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951]. Previous work demonstrated a 0% survival rate in mice immunized with B. pseudomallei-pulsed dendritic cells, though the immunization generated a substantial cell-mediated immune response [Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951]. Protection could be achieved when the mice were boosted with heat-killed bacteria, and correlated with the production of high B. pseudomallei-specific antibody titers [[Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951]. Immunization with EF-Tu yielded high concentrations of antigen-specific IgG in the sera and bronchoalveolar lavage (BAL) by both the parenteral and mucosal immunization regimens. However, EF-Tu-specific IgG levels did not correlate with the observed differences in lung bacterial burdens in immunized mice in the instant study. In addition to IgG, secretory IgA may play a role in protection against inhalational pathogens as previously demonstrated for Bordetella pertussis Watanabe M, Nagai M (2003) Role of systemic and mucosal immune responses in reciprocal protection against Bordetella pertussis and Bordetella parapertussis in a murine model of respiratory infection. Infect Immun 71: 733-738]. Undetectable to very low levels of EF-Tu-specific IgA were observed in the sera of immunized mice regardless of the route of immunization. In contrast, EF-Tu-specific IgA was significantly elevated in the BAL of immunized mice compared to naïve mice, but there was no statistical difference among any of the immunized groups. Therefore, IgA concentrations also may not account for the differences observed in lung bacterial burdens at the time point examined.

Although antibodies contribute to protection against B. pseudomallei [Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951] a robust CMI response is likely required for ultimate clearance of internalized bacteria [Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951; Haque A, Easton A, Smith D, O'Garra A, Van Rooijen N, et al. (2006) Role of T cells in innate and adaptive immunity against murine Burkholderia pseudomallei infection. J Infect Dis 193: 370-379]. Antigen-specific T cells, particularly CD4+ T cells, are important sources of interferon-gamma (IFN-γ) and are essential for host resistance to acute and chronic infection with B. pseudomallei [Haque A, Easton A, Smith D, O'Garra A, Van Rooijen N, et al. (2006) Role of T cells in innate and adaptive immunity against murine Burkholderia pseudomallei infection. J Infect Dis 193: 370-379.]. EF-Tu was recently shown to elicit memory CD4+ T cells in cattle immunized with outer membrane protein preparations of the rickettsial pathogen, Anaplasma marginale [Lopez J E, Beare P A, Heinzen R A, Norimine J, Lahmers K K, et al. (2008) High-throughput identification of T-lymphocyte antigens from Anaplasma marginale expressed using in vitro transcription and translation. J Immunol Methods 332: 129-141]. The instant disclosure corroborates those findings as it demonstrates both Th1 (IFN-γ) and Th2 (IL-5) cytokine production in EF-Tu-restimulated splenocytes that reflected both the adjuvant used and the route of immunization. In other words, the parenteral immunization strategy that incorporated aluminum hydroxide as adjuvant promoted Th2 responses to rEF-Tu, while the mucosal administration of rEF-Tu with CpG polarized the immune response towards Th1. This is also supported by the IgG1:IgG2a ratios observed in the sera and BAL that demonstrated a Th1 polarization in mucosally immunized mice (TABLE 2). It is plausible that the antigen-specific Th1 response elicited by mucosal immunization with rEF-Tu/CpG is responsible for the reduced bacterial burden observed early in the lungs of the i.n.+i.n. group. Considering the predominance of EF-Tu in the bacterial cell, further analysis of EF-Tu-specific CD4+ T memory cells is clearly warranted.

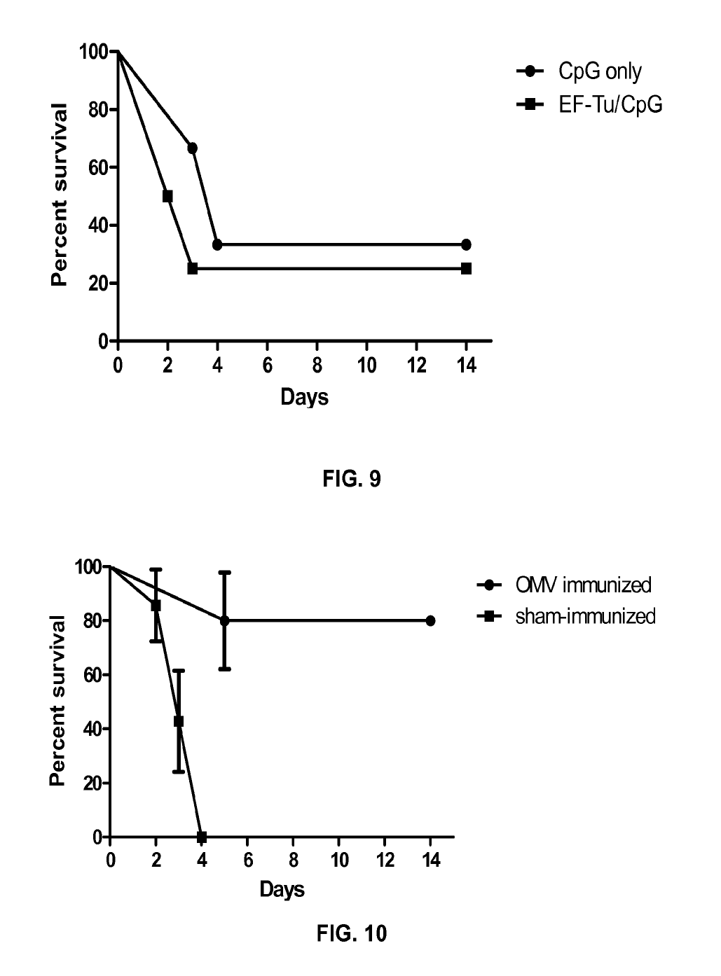

The applicant was able to demonstrate EF-Tu, identified as a candidate immunogen, yields a robust IgG response and some IgA, produces stimulation of Th1 and Th2 cells (as measured by IFN-α, and IL-5, respectively), and reduces bacterial burden (FIG. 6), yet immunization with EF-Tu does not confer protection from B. pseudomallei (FIG. 9).

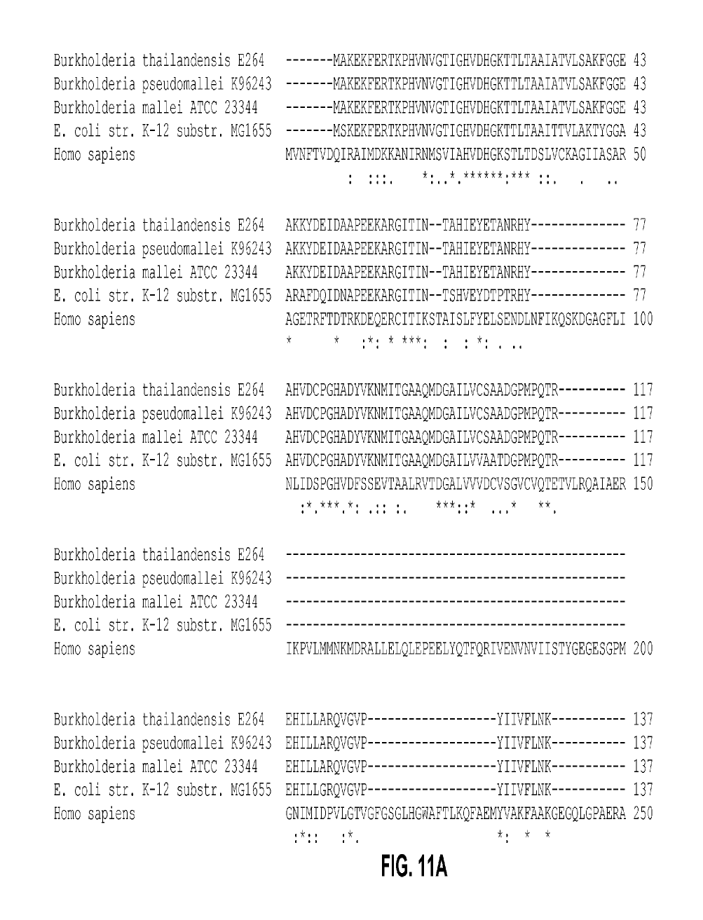

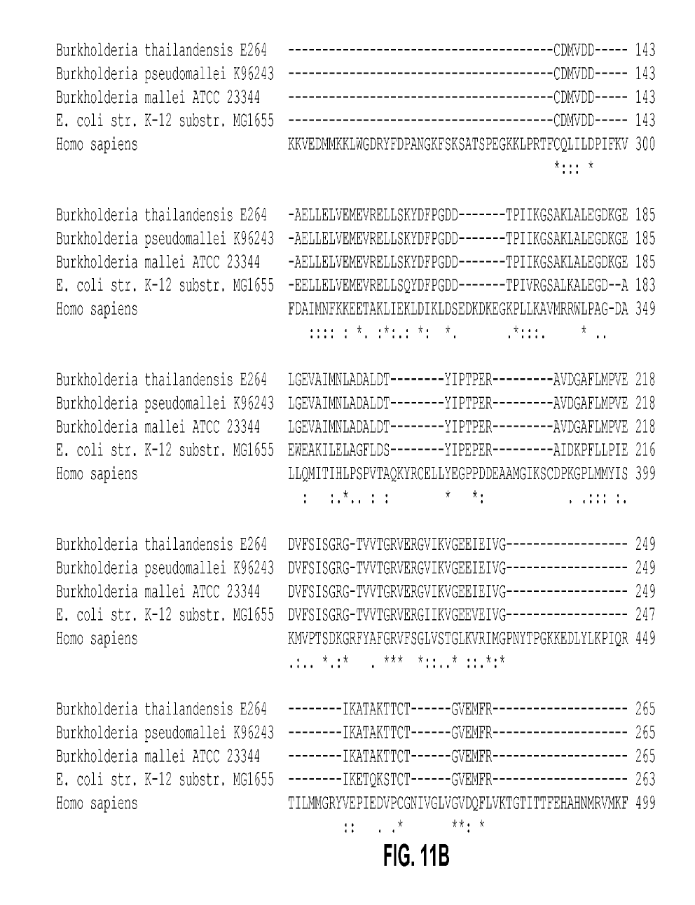

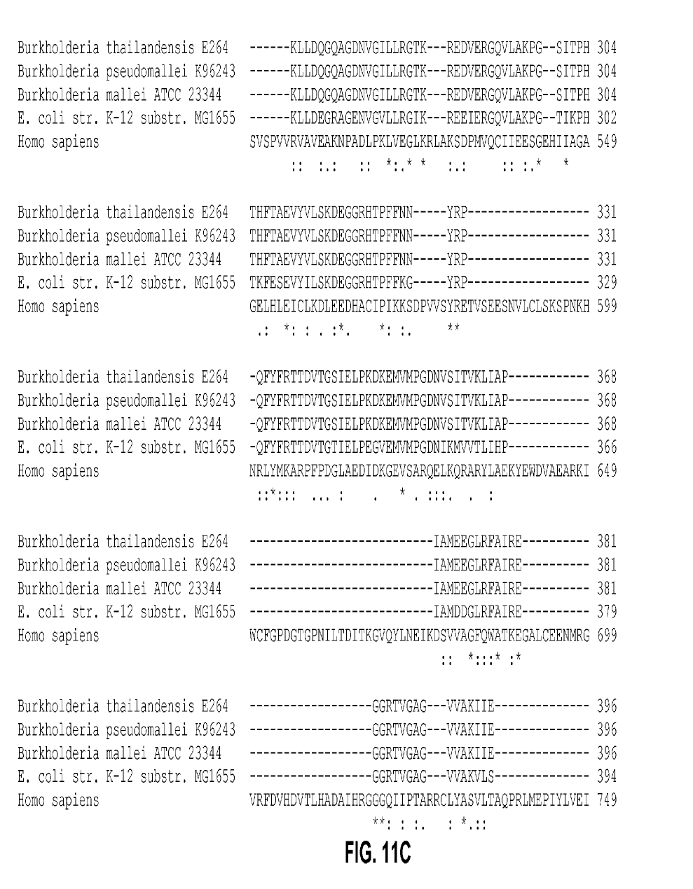

The applicant was able to demonstrate protection of mice immunized subcutaneously with 2.5 μg of purified Bp OMVs resuspended in 100 μL of saline on days 0, 21, and 42 (FIG. 10). Sham-immunized mice received 100 μL saline subcutaneously. All mice were challenged on day 70 with a lethal dose of B. pseudomallei (500 cfu) by aerosol, and survival was monitored for 14 days. One hundred percent (100%) of the sham-immunized mice succumbed to challenge within 4 days, while 80% of the OMV-immunized mice survived until the study endpoint and appeared to have completely recovered as determined by normal behavior/activity and confirmed absence of bacteria in the lungs (FIG. 10).

The lack of adequate treatment and prevention against melioidosis necessitates the development of a vaccine against B. pseudomallei [Bondi S K, Goldberg J B (2008) Strategies toward vaccines against Burkholderia mallei and Burkholderia pseudomallei. Expert Rev Vaccines 7: 1357-1365]. Inhalation of B. pseudomallei is a natural route of infection, and it represents the primary route of exposure in a deliberate biological attack. A B. pseudomallei vaccine should therefore be efficacious against this route of infection. EF-Tu, a protein best recognized for its role in bacterial protein synthesis, was identified as a subunit vaccine candidate against pathogenic Burkholderia. The data presented here indicate that recombinant EF-Tu is immunogenic, inducing antigen-specific antibody and CMI responses, yet FIG. 9 demonstrates that immunization with EF-Tu did not confer complete protection against Burkholderia. Rather, OMV prepared from Burkholderia conferred protection against aerosolized Burkholderia. Burkholderia mallei, the etiologic agent of glanders disease, is a Gram-negative, non-motile, facultative intracellular bacterium. Most known members of the family Burkholderiaceae are resident in the soil; however, B. mallei is an obligate mammalian pathogen. Horses are highly susceptible to infection and are considered to be the natural reservoir for infection, although mules and donkeys are susceptible as well (Neubauer H. et al., J Vet Med B Infect Dis Vet Public Health, 52: 201-205 (2005), the disclosure of which is incorporated herein by reference). Identification of the etiologic agent B. mallei was described in 1882 by isolating an organism from the infected liver and spleen of a horse

Clinically, B. mallei infected solipeds can present with either a chronic (horses) or an acute (mules and donkeys) form. Although eradication has been successful in the United States, glanders is endemic among domestic animals in Africa, Asia, the Middle East and Central and South America. The primary route of equine infection is most likely the consumption of feed or water contaminated with nasal discharges of infected animals, although a cutaneous form also exists, known as farcy. Chronically infected animals present a variety of signs and symptoms dependent on the route of infection including mucopurulent nasal discharge, lung lesions and nodules involving the liver and spleen. Acute infection results in high fever and emaciation, with ulceration of the nasal septum, accompanied by mucopurulent to hemorrhagic discharge. Pathological changes are limited in gut-associated lymphatic tissues, with the majority of pathology occurring in the lungs and airways

In an embodiment of the invention, the present immunogenic compositions are used in methods of protecting a horse, mule, or donkey subject against infection caused by at least one species of Burkholderia, wherein administration of the immunogenic composition provides protection against infection.

In another embodiment of the invention, the present immunogenic compositions are used in methods of inducing an immune response to at least one species of Burkholderia in a horse, mule, or donkey subject, said method comprising administering the immunogenic composition in an amount effective to elicit production of antibodies specific to the at least one species of Burkholderia.

In yet another embodiment of the invention, the present immunogenic compositions are used in methods of preventing respiratory infection in a horse, mule, or donkey subject wherein the respiratory infection is caused by at least one species of Burkholderia, wherein administration of the immunogenic composition prevents at least one symptom of said respiratory infection.

In yet another embodiment of the invention, the present immunogenic compositions are used in methods of preventing meliodosis in a subject wherein the meliodosis is caused by at least one species of Burkholderia, wherein administration of the immunogenic composition produces immunity in the subject when the subject is subsequently exposed to said at least one species of Burkholderia, and wherein administration of the immunogenic composition prevents at least one symptom of said meliodosis.

### Optional Vaccine Components

The present disclosure provides a vaccine composition comprising outer membrane vesicles without additional vaccine components traditionally utilized in immunization strategies. However, components can optionally be added that function to stabilize the composition or provide a balanced immune reaction. These components include but are not limited to lipopolysaccharide (LPS), CpG, aluminum hydroxide adjuvant, and saline.

### Experimental Methods

### Two-Dimensional Gel Electrophoresis

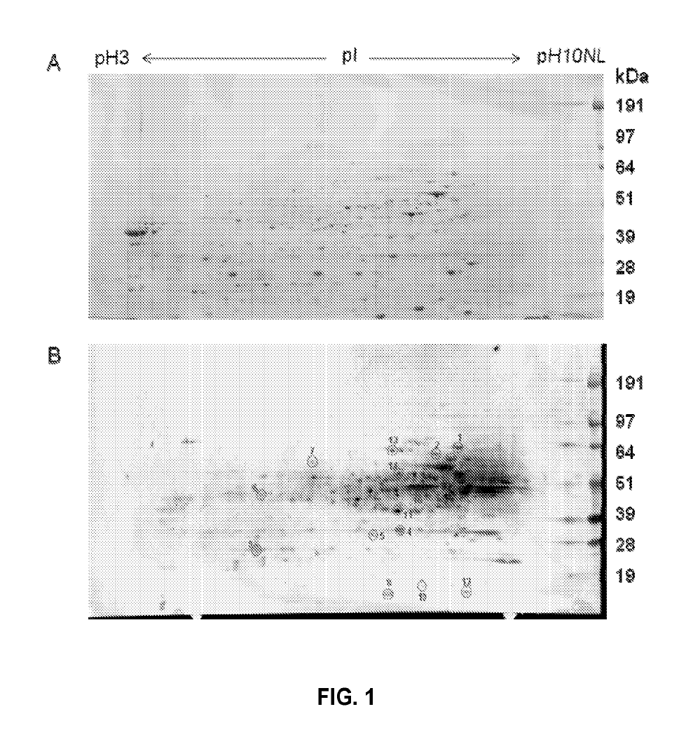

Two-dimensional (2D)-gel electrophoresis was performed using 100 μg of B. thailandensis whole cell lysate solubilized in 7 M urea, 2M thiourea, 4% (w/v) 3-[3-(cholamidopropyl)-dimethylammonio]-1-proanesulphonate (CHAPS), 20% glycerol, 30 mM Tris, pH 8.5. Fifty μg (50 μg) of the crude lysate was used to rehydrate an 18 cm immobilized pH gradient (IPG) strip, pH 3-10 non-linear (NL) overnight. The following day, the proteins in the rehydrated strip were subjected to isoelectric focusing at 50 μA/strip. The strip was then equilibrated 15 min with 20 mg/ml dithiothreitol (DTT) and 25 mg/ml iodoacetamide before loading onto a 12.5% SDS-polyacrylamide gel (Invitrogen). The gel was run for 30 min at 5 Watts/gel and then for 5 hr at 18 Watts/gel. Western blot was performed as described below with a few modifications: the membrane was blocked with 5% skim milk in TBS containing 0.05% Tween 20 (TBST); a 1:200 dilution of polyclonal serum from New Zealand White rabbits that were immunized subcutaneously with irradiated B. mallei ATCC 23344 was used as the primary antibody; and a 1:2000 dilution of a goat anti-rabbit HRP-conjugated antibody was used as the secondary. See, e.g., FIG. 1.

Matrix assisted laser desorption ionization time of flight (MALDI-TOF) mass spectrometry MALDI-TOF analysis was performed on a 4700 Proteomics Analyzer MALDI-TOF-TOF (Applied Biosystems, Foster City, Calif.). An averaged simple mass spectrum and tandem mass spectra from the five most abundant peptides (excluding trypsin autolysis) of each sample were acquired and manually inspected in Data Explorer. Global Proteome Server (Applied Biosystems) was utilized to search the bacteria of Uniprot protein database. One missed cleavage per peptide was allowed, and the fragment ion mass tolerance window was set to 100 ppm. A protein hit with a total score of 75 or higher, with at least one peptide over 20, was considered a likely match. Protein similarities were obtained using Basic Local Alignment Search Tools (BLAST) (http://www.ncbi.nlm.nih.gov/BLAST) and the NCBI non-redundant database.

### Cloning, Expression, and Purification of EF-Tu

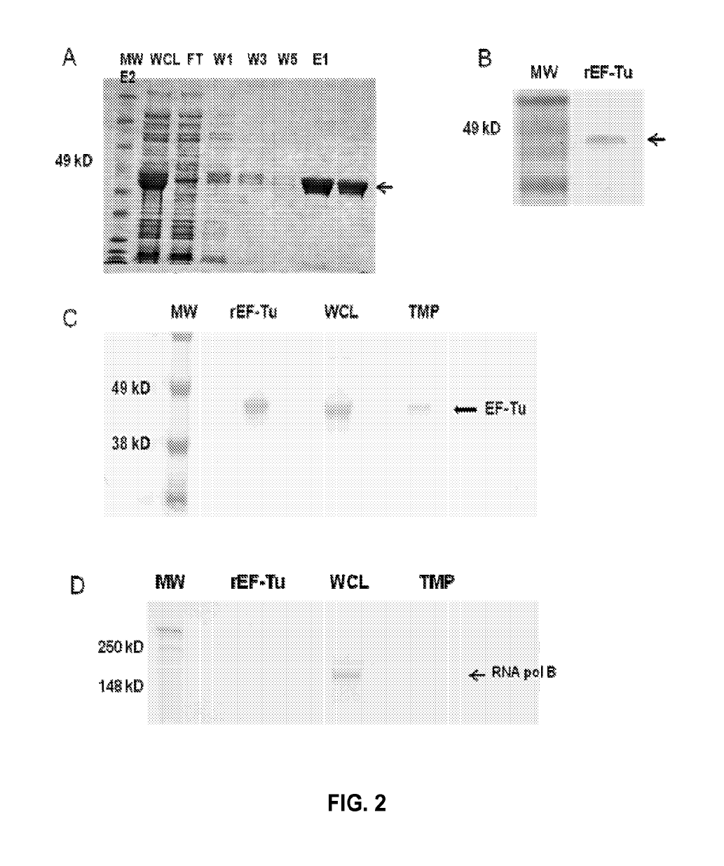

Based on the published genome sequence of B. pseudomallei strain K96243, the complete open reading frame (ORF) of EF-Tu was PCR amplified from B. pseudomallei strain 286 genomic DNA (BEI Resources, Manassas, Va.) using the forward primer 5′-GCATGCGCCAAGGAAAAGTTTGAGCGGACC-3′ (SEQ ID NO:1) and the reverse primer 5′-AAGCTTTTACTCGATGATCTTGGCGACGACG-3′ (SEQ ID NO:2) which produces SphI and HindIII sites (underlined) at the 5′- and 3′-ends of the EF-Tu ORF respectively. The fragment was ligated into the multi-cloning site of the protein expression vector pQE30 (Qiagen, Valencia, Calif.) containing an N-terminal 6×-histidine tag, and transformed into E. coli strain DH-5α for automated sequencing using the pQE forward and reverse sequencing primers (Qiagen). The cloned EF-Tu from strain 286 shares 100% amino acid sequence identity with EF-Tu from B. pseudomallei strain K96243 (Uniprot/Swiss prot # Q63PZ6) and B. thailandensis E264 (Uniprot/Swiss prot # Q2SU25) and 79.4% identity with E. coli K12 (Uniprot/Swiss prot # P0CE48). For over-expression of the EF-Tu protein, the construct was transformed into E. coli strain M15 (Qiagen) and transformants were cultured overnight at 37° C. in Luria-Bertani (LB) broth supplemented with ampicillin (100 μg/ml) and kanamycin (50 μg/ml). A 1:100 dilution was used to inoculate fresh LB broth supplemented with ampicillin (50 μg/ml) and kanamycin (25 μg/ml) and allowed to grow to mid-log phase before induction with 1 mM isopropyl-β-d-thiogalactoside (IPTG) for 4 hr. Cells were harvested by centrifugation and the cell pellet was stored at −80° C. overnight. Cells were resuspended in lysis buffer (50 mM NaH2PO4, 300 mM NaCl, 10 mM imidazole), and sonicated three times for 30 sec. Supernatant containing recombinant EF-Tu (rEF-Tu) protein was collected after centrifugation, and simple batch purification was achieved using Ni-NTA agarose beads (Qiagen) under native conditions. Agarose beads were washed three times with buffer containing 20 mM imidazole, five times with 0.5% amidosulfobetaine-14 (ASB-14) to remove lipopolysaccharide (LPS), five times with 20 mM Tris-HCl, and eluted with 250 mM imidazole. Eluted protein fractions were concentrated by centrifugation (Amicon, MW cutoff 10,000 kDa), and imidazole was removed by buffer exchange with LPS-free water. LPS contamination was determined to be less than 25 EU/ml using the limulus amebocyte lysate (LAL) assay (Lonza, Switzerland). Protein concentration was determined using the Bradford protein assay (BioRad). See, e.g., FIGS. 2A-2D.

Total membrane protein extraction, SDS-PAGE, and Western blot.

A single colony of either B. thailandensis or E. coli was used to inoculate LB broth and incubated overnight. Each culture was freshly diluted 1:100 into LB broth the next morning. The bacterial cells were grown to log-phase and harvested by centrifugation (6,000×g, 10 min, 4° C.). The bacterial pellet was resuspended in 1/50th volume of 4-(2-hydroxyethyl)-1-piperazineethanesulfonic acid (HEPES) buffer (10 mM, pH 7.4). Lysozyme was added at a final concentration of 10 mg/ml and incubated for 20 min at room temperature. The bacterial suspension was sonicated five times (50-Watts) for 30 sec each on ice. Benzonase (Novagen, Gibbstown, N.J.) was added at a final concentration of 1 μg/ml, and the lysate was incubated for 30 min at room temperature. Intact cellular debris was removed by centrifugation (12,000×g, 10 min, 4° C.). A sample of the supernatant consisting of the whole cell lysate was stored at −80° C. until use. The remaining supernatant was centrifuged (50,000×g, 60 min, 4° C.), and the resulting pellet was resuspended in 0.5% Sarkosyl (Sigma) and incubated 30 min at room temperature. The suspension consisting of total membrane proteins (both inner and outer membrane) was aliquoted and stored at −80° C. until use.

Sodium dodecyl sulphate polyacrylamide gel electrophoresis (SDS-PAGE) was performed using a 4-20% polyacrylamide gel (Bio-Rad). rEF-Tu or proteins from whole cell lysate or total membrane fractions of either B. thailandensis or E. coli were separated under reducing conditions, and the proteins were subsequently transferred to nitrocellulose membranes. The membranes were blocked with 1.5% BSA in TBST overnight at 4° C. and then washed twice with TBST. The membranes were then incubated overnight at 4° C. with pooled sera (1:200 dilution) from rEF-Tu immunized mice; pooled sera (1:200 dilution) obtained from mice 2 weeks after intraperitoneal (i.p.) challenge with 107 cfu B. thailandensis strain E264 (American Type Culture Collection (ATCC), Manassas, Va.); pooled (pre-immune) sera (1:200 dilution) from naïve mice; or with a monoclonal antibody (1:1000 dilution) to the β subunit of E. coli RNA polymerase (Neoclone, Madison, Wis.). The RNA polymerase antibody did not recognize B. thailandensis and was therefore used only on E. coli cellular fractions to determine the purity of total membrane preparations. The membranes were subsequently washed three times with TBST and incubated with goat anti-mouse HRP-conjugated secondary antibody (1:1000 dilution) (Thermo Scientific Pierce, Rockford, Ill.) for 1 hr at room temperature. The membranes were washed twice with TBST and developed with Opti-4CN Substrate (BioRad, Hercules, Calif.).

### Outer Membrane Vesicle (OMV) Preparation

OMVs were prepared as previously described [Moe G R, Zuno-Mitchell P, Hammond S N, Granoff D M (2002) Sequential immunization with vesicles prepared from heterologous Neisseria meningitidis strains elicits broadly protective serum antibodies to group B strains. Infect Immun 70: 6021-6031; Bauman S J, Kuehn M J (2006) Purification of outer membrane vesicles from Pseudomonas aeruginosa and their activation of an IL-8 response. Microbes Infect 8: 2400-2408] with minor modifications. B. pseudomallei strain 1026b (BEI Resources) was grown in LB broth at 37° C. until late log phase (16-18 hr). The intact bacteria were pelleted by centrifugation at 6,000×g for 10 min at 4° C., and the supernatant was removed and filtered twice through a 0.22 μm polyethersulfone (PES) filter (Millipore) in order to remove any remaining bacteria or large bacterial fragments. To ensure the supernatant was free of viable bacteria, 1 mL of supernatant was streaked onto PIA agar and incubated 48-72 hrs at 37° C. The remaining filtered supernatant was incubated at 4° C. OMVs were harvested by slowly adding 1.5 M solid ammonium sulfate (Fisher Scientific) while stirring gently and incubated overnight at 4° C. The OMVs were harvested by centrifugation at 11,000×g for 20 min at 4° C. The resulting pellet, consisting of crude vesicles, was resuspended in 45% OptiPrep (Sigma) in 10 mM HEPES/0.85% NaCl, pH 7.4, filter sterilized through a 0.22 μm PES filter and layered at the bottom of a centrifuge tube. An OptiPrep gradient was prepared by slowly layering 40%, 35%, 30%, 25%, and 20% OptiPrep in HEPES-NaCl (w/v) over the crude OMV preparation. Membrane vesicles were collected by ultracentrifugation at 111,000×g for 2 hr at 4° C. Equal fractions were removed sequentially from the top and stored at 4° C. To determine the purity of the fractions, 250 μL of each was precipitated with 20% (w/v) Tri-chloroacetic acid (TCA). The resulting pellet was resuspended in 10 μL phosphate buffered saline (PBS) and 10 μL Laemmli loading buffer (Bio-Rad), boiled for 10 min and loaded onto an SDS-PAGE polyacrylamide gel (4-20% Mini Protean, Bio-Rad) run at 200 V. The working OMV preparation was recovered by pooling the peak fractions (those containing the least amount of insoluble fragments and contaminants) in 50 mM HEPES, pH 6.8 followed by centrifugation at 111,000×g for 2 hr at 4° C. The resulting pellet containing OMVs was resuspended in LPS-free water (Lonza) and stored at −20° C. OMVs were quantified with a Bradford Protein Assay (Bio-Rad). Cryo-Transmission Electron Microscopy was performed using a JEOL 2010 transmission electron microscope to visually confirm the presence of OMVs.

### Animals

Female BALB/c mice 8- to 10-weeks-old were purchased from Charles River Laboratories (Wilmington, Mass.) and maintained 5 per cage in polystyrene microisolator units under pathogen-free conditions. Animals were fed sterile rodent chow and water ad libitum and allowed to acclimate 1 week prior to this study. Mice were euthanized by carbon dioxide overdose.

### Bacterial Challenges

With respect to the present disclosure, the phrase “lethal dose” as used herein is intended to refer to any dosage amount that can cause lethality in a subject. In a preferred embodiment of the invention, the present immunogenic compositions are used to protect a subject against lethal doses of at least one species of Burkholderia. It is well understood that the exact dosage amount depends on a variety of factors, including, the particular species of Burkholderia, the route of infection, and the immune and/or health state of the subject. For instance, it is well understood that aerosol exposure to Burkholderia is more lethal to a human subject than when Burkholderia is ingested in the same human subject. Thus, the lethal dose of aerosolized Burkholderia will be less than the lethal dose for ingested Burkholderia in the same human subject. Likewise, it is well understood than immune compromised subjects will succumb to lower doses of the same Burkholderia in comparison to healthy, non-immune compromised subjects. Exemplary amounts of lethal doses of Burkholderia range from 1 c.f.u. to about 108 c.f.u.

### Bacterial Challenges—Intraperitoneal (i.p.)

Prior to murine challenge, B. thailandensis was freshly grown from frozen glycerol stock in LB broth overnight and freshly diluted 1:100 into LB broth the next morning. The bacteria were grown to log-phase and harvested by centrifugation and diluted into 0.9% NaCl to 1×108 colony forming units (cfu)/ml. Each mouse (N=6) was administered 100 μL of bacteria (107 cfu) via the i.p. route. Mice were monitored for symptoms of illness twice daily for 14 days and survivors were euthanized at the end of study. Blood samples were collected via cardiac puncture following euthanasia. Blood was allowed to clot for 30 min at room temperature and then centrifuged at 2300×g; serum was collected and stored at −80° C. until use.

### Bacterial Challenges—Aerosol

BALB/c mice were challenged with 5×105 cfu (˜LD50) of B. thailandensis or 500 cfu (LD100) of B. pseudomallei using a nose-only inhalation exposure chamber as previously described [West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17]. For determination of lung bacterial burden, mice were euthanized at 24 hr post-challenge.

### EF-Tu Immunizations

BALB/c mice (N=70) were primed subcutaneously (s.c.) on day 0 with 25 μg of purified rEF-Tu in LPS-free water adsorbed 1:1 with aluminum hydroxide adjuvant (Alhydrogel 2%, Brenntag, Germany) in a final volume of 100 μL or intranasally (i.n.) with 25 μg rEF-Tu in LPS-free water admixed with 5 μg CpG oligodeoxynucleotide (ODN) 1826 adjuvant (Coley, Wellesley, Mass.) in a final volume of 9 μL/nostril. Prior to intranasal immunization, mice were anesthetized via the i.p. route with 0.88 mg/kg ketamine/xylazine in saline in a final volume of 100 μL. Mice were boosted on day 21 with the same formulations using a homologous (s.c.+s.c. or i.n.+i.n.) or heterologous (s.c.+i.n.) prime/boost strategy.

### OMV Immunizations

Mice were immunized subcutaneously with 2.5 μg of purified B. pseudomallei (Bp) OMVs resuspended in 100 μL of saline on days 0, 21, and 42. Sham-immunized mice received 100 μL saline subcutaneously on the same schedule. All mice were challenged on day 70 with a lethal dose of B. pseudomallei (500 cfu) by aerosol and survival was monitored for 14 days. One hundred percent (100%) of the sham-immunized mice succumbed to challenge within 4 days, while 80% of the OMV-immunized mice survived until the study endpoint and appeared to have completely recovered as determined by normal behavior/activity and confirmed absence of bacteria in the lungs.

### Analysis of Antibody Response

Blood samples from immunized and naive mice were collected via cardiac puncture following euthanasia for determination of rEF-Tu specific serum antibody concentration. Blood was allowed to clot for 30 min at room temperature and then centrifuged at 2300×g; serum was collected and stored at −80° C. until assayed. Bronchoalveolar lavage (BAL) fluid was collected for determination of rEF-Tu specific BAL antibody concentration. BAL fluid was obtained by exposing the trachea and making a small incision into which an 18-gauge needle was inserted and secured. The lungs were repeatedly lavaged by slowly injecting and withdrawing 1 ml of phosphate buffered saline (PBS) supplemented with Complete protease inhibitor cocktail (Roche Laboratories, Mannheim, Germany). BAL fluid was stored at −80° C. until assayed. The concentrations of serum and BAL fluid rEF-Tu-specific total IgG, IgG1, IgG2a, and IgA were analyzed by enzyme-linked immunosorbent assay (ELISA). Ninety-six-well microtiter plates were coated with 0.5 μg per well of purified rEF-Tu in coating buffer (0.1 M sodium bicarbonate, 0.2M sodium carbonate) and incubated overnight at 4° C. The plates were washed three times with PBS containing 0.05% Tween-20 (PBST). For measurement of IgA, plates were additionally blocked with 2% BSA for 1 hr followed by three washes with PBST. All plates were incubated with two-fold serial dilutions of sera or BAL samples for 2 hr at room temperature. Plates were washed three times with PBST and then incubated with either alkaline phosphatase (AP)-conjugated rat anti-mouse IgG, IgG1, IgG2a (1:300 dilution in PBST) (BD Pharmingen) or AP-conjugated goat-anti-mouse IgA (1:2000) (Invitrogen) for 1 hr at room temperature. At the end of the incubation, the plates were washed three times with PBST and developed with SIGMAFAST p-Nitrophenyl phosphate tablets (Sigma, St. Louis, Mo.) dissolved in diethanolamine buffer (1 mg/ml). After 15-30 min of incubation, reaction solutions were stopped with 2 M NaOH and read at 405 nm using μQuant microplate reader and analyzed with Gen5 software (BioTek, Winooski, Vt.). Antibody concentrations were determined by non-linear regression from a standard curve of mouse myeloma IgG1, IgG2a, and IgA (Sigma) serially diluted as a standard on each ELISA plate [Glynn A, Freytag L C, Clements J D (2005) Effect of homologous and heterologous prime-boost on the immune response to recombinant plague antigens. Vaccine 23: 1957-1965.]. The results obtained are expressed as the mean concentration±standard error of the mean (SEM).

### Antigen Restimulation Assay

Restimulation assays were performed with splenocytes from immunized and naïve mice for analysis of T cell responses. Spleens were removed aseptically and single-cell splenocyte suspensions from each mouse were obtained by passing the spleens through sterile 40 μm cell strainers (Fisher Scientific; Pittsburgh, Pa.). Cells were washed twice with wash buffer (Advanced RPMI 1640 medium supplemented with 1% fetal bovine serum (FBS) and 1% antibiotic-antimycotic) (Invitrogen). Cell pellets were resuspended in wash buffer and layered onto Histopaque-1119 (Sigma) for splenic mononuclear leukocyte isolation by centrifugation at 300×g for 15 min. Leukocytes were recovered at the interface and washed twice with wash buffer and resuspended in Advanced RPMI 1640 medium supplemented with 10% FBS and 1% antibiotic-antimycotic. Cells were plated in a 96-well microtiter plate at 4×105 cells/well. Cell cultures were stimulated with 1 μg of rEF-Tu, 1 μg concanavalin A (ConA) (Sigma), or left unstimulated as negative controls. The cultures were incubated at 37° C. in 5% CO2, and cell culture supernatants from each treatment group were collected after 72 hr and stored at −80° until use.

### CFU Recovery

Lung tissue homogenates were used to determine bacterial burden in aerosol-infected mice. Lungs were aseptically removed, weighed, and individually placed in 1 ml 0.9% NaCl and homogenized with a Power Gen 125 (Fisher Scientific). Ten-fold serial dilutions of lung homogenates were plated on LB agar. Colonies were counted after incubation for 2-3 days at 37° C. and reported as cfu per gram of tissue.

### Statistical Analyses

All analyses were performed using GraphPad Prism version 5.0 (GraphPad Software, Inc., La Jolla, Calif.). Statistical analysis of cytokine production was performed using a two-way ANOVA, and analyses of antibody concentrations and bacterial burdens were performed using the Mann-Whitney test. Values of P<0.05 were considered statistically significant.

### Detailed OMV Purification Protocol

The purpose of this protocol is to extract B. pseudomallei OMV and eliminate other contaminants such as LPS, whole cell bacteria and cellular fragments with the aid of the OptiPrep gradient buffer. Filter sterilization was used to eliminate whole cell bacteria or large bacterial fragments. Ammonium sulfate precipitation was used as the preferred method for precipitating OMVs out of solution. The protocol was adapted from Moe, et al. 2002 (Infect. Immun. Vol. 70 No 11), Bauman and Khuen 2006 (Microbes and Infection 8 2400e2408) and Horstman and Khuen 2000 (J Biol. Chem. Vol. 275 No. 17).

This protocol is intended for a 500 mL culture supernatant, which should yield ˜0.2 mg/mL OMV in a 300 μL to 500 μL total volume. The best yield of OMVs was achieved with a total 1 L culture supernatant.

Day 1: Grow a 5 mL culture of B. pseudomallei (Bp) overnight. Obtain one colony of B. pseudomallei grown on a PIA plate (streaked from glycerol stock) to inoculate 5 mL LB broth. Grow overnight (O/N), 37° C., 233 rpm.

Day 2: Do a 1:100 dilution of the O/N Bt culture into 495 mL of LB broth. Grow for 16 hours to late log phase-early stationary phase (OD ˜6.0), 37° C., 233 rpm.

Day 3: (a) Pellet the whole B. pseudomallei cells by centrifuging 6,000×g (6,300 rpm), 10 min, 4° C.; store the bacterial pellets at −80° C. (as needed) for extraction of WCL, TMP and OMP as previously described; the supernatant contains the OMV; repeat this step (a) one more time to ensure there are no bacteria in the supernatant.

Day 3: (b) Filter the supernatant through a 0.22 μm (sterile filtration) Millipore PES filter (Cat # SCGPU10RE) to remove any remaining bacteria or large bacterial fragments. Repeat once to ensure there are no bacteria in the supernatant.

Day 3: (c) Harvest the membrane vesicles in the filtered supernatant by slowly adding 1.5 M solid ammonium sulfate ((NH2)4SO4) while slowly stirring. Incubate at 4° C. overnight. The vesicles will precipitate along with other contaminants (precipitate is a dark brown color). Obtain 1 mL from Day 3 step (c) and plate onto PIA agar. Incubate O/N, 37° C. There should be no growth. Allow plate to stay in incubator up to 48 hrs (if needed) in order to prove there is no bacterial growth.

Day 4: (a) Make sure there is no growth in the PIA plate. Bacterial growth is an indicator of bacterial contamination. (If no growth, proceed with OMV extraction).

Day 4: (b) Pellet the OMVs by centrifugation at 11,000×g (8,500 rpm), 20 min, 4° C. using an SLA-1500 rotor (Sorvall); gently resuspend the dark brown pellets and the OMV smear along the side of the centrifuge tube in 45% OptiPrep (Sigma) in 10 mM HEPES/0.85% NaCl, pH 7.4 (HEPES-NaCl weight/volume) in a 4 mL total volume.

Day 4: (c) To remove any lumps of the pellet, filter sterilize through a 0.45 um filter (Millipore, 50 ml conical tube filter system) as previously described. This is the crude vesicle preparation.

Day 4: (d) To obtain debris-free OMV preparation: An OptiPrep gradient is prepared as followed: Layer on the bottom of a 26.3 mL centrifuge bottle (Beckman Coulter, 355618) the 4 mL of crude OMV from step d. Then very gently and slowly layer over 4 mL 40%, 4 mL 35%, 6 mL 30%, 4 ml 25%, and 4 ml of 20% OptiPrep in HEPES-NaCl (w/v). The differences in the gradients reflect optimization in separating flagella and other soluble material from the vesicles. Ultracentrifuge the gradients to collect the membrane vesicles, using a Beckman Coulter Ultracentrifuge, Rotor Type 52.1 Ti, 111,000×g (35,000 rpm) for 2 hr, 4° C.

Day 4: (e) Four mL (4 mL) fractions are gently and sequentially removed from the top, and stored in 15 ml tubes at 4° C. (or continue to the next steps: Analysis of OMV Fractions for Purity, and TCA Protocol).

### Analysis of OMV Fractions for Purity

A portion of each OMV fraction (˜1 mL from each fraction from the OMV purification protocol, above) was taken to precipitate the OMVs with 20% Tri-chloroacetic Acid (TCA). The precipitated OMVs can be visualized by western blotting, Coomasie or silver stained gels.

### TCA Protocol

Stock Solutions: 20% (w/v) Tri-chloroacetic acid (TCA); 1) Add 1 volume of 20% TCA to 4 volumes of protein sample (i.e., in 1.5 ml tube with maximum vol., add 125 μl 20% TCA to 1.5 ml sample while working on ice); 2) Incubate 10 min at 4° C.; 3) Spin tube in micro-centrifuge at 13,000 rpm, 5 min, RT; 4) Remove supernatant, leaving pellet intact. Pellet should be formed from whitish, fluffy ppt; 5) Wash pellet with 200 μl cold acetone; 6) Spin tube in micro-centrifuge at 13,000 rpm, 5 min; 7) Repeat steps 4-6 for a total of 2 acetone washes; 8) Dry pellet for 5-10 min to drive off acetone. The white pellet may become translucent; 9) For SDS-PAGE, Resuspend pellet in 20 μL Laemlli loading sample buffer containing beta-mercapto-ethanol (or 100 mM DTI) and boil sample for 7 min. Allow the sample to cool down, do a quick spin and load all 20 μL onto a poly-acrylamide gel as previously described for Coomasie staining; 10) Pick the fractions with the least amount of contaminants or insoluble material. Desired fractions were pooled and concentrated in a 100 kD desalting column (Milipore) by centrifuging at 2300×g, 25 min, 4° C. A final spin was performed using 2 ml LAL LPS-free water to remove any residual Opti-Prep reagent. The final OMV vaccine preparation is in LPS-free water and stored at −20° C. in aliquots to prevent frequent freeze/thaw cycles.

### Example 1

### Identification of EF-Tu as a Potential Vaccine Candidate for B. pseudomallei

An immunoproteomic approach [Rappuoli R (2000) Reverse vaccinology. Curr Opin Microbiol 3: 445-450] was employed to identify novel immunogenic Burkholderia proteins that could be further screened for their ability to elicit both antibody and CMI responses. At that time, antisera against B. pseudomallei was not available. Therefore, pooled antisera from B. mallei-immunized rabbits was used to probe a B. thailandensis whole cell lysate that was separated by 2D-gel electrophoresis (FIG. 1A, showing SYPRO-ruby stained gel). It was the hypothesis that proteins shared by B. mallei, B. pseudomallei, and B. thailandensis could be detected by this approach due to the extensive homology between the three species [Kim H S, Schell M A, Yu Y, Ulrich R L, Sarria S H, et al. (2005) Bacterial genome adaptation to niches: divergence of the potential virulence genes in three Burkholderia species of different survival strategies. BMC Genomics 6: 174]. The immunoblot revealed more than 100 immunoreactive proteins of which we randomly selected 16 spots for identification by MALDI-TOF mass spectrometry (FIG. 1B, showing Western blot performed using rabbit anti-B. mallei polyclonal sera (1:200 dilution), followed by HRP-conjugated goat anti-rabbit IgG (1:2000) and detected with Opti-4CN substrate (BioRad)). None of the selected spots were detected using antisera from naïve rabbits. Eleven proteins were successfully identified and share 96-100% amino acid identity among the three Burkholderia species (Table 1). TABLE 1 shows putative function and percent identity to B. pseudomallei K96243 and B. mallei ATCC 23344 at the amino acid level. Proteins shown in boldface type in TABLE 1 were also identified by Harding et al. using a similar approach; “*” indicates unidentified by mass spectrometry; “Not present” indicates that no known ortholog is annotated in the NCBI genomic database.

TABLE 1
Immunoreactive B. thailandensis proteins identified by MALDI-TOF mass spectrometry

| | | | | | | % identity to | % identity to |
|---|---|---|---|---|---|---|---|
| Sample | | Accession | | Theoretical | Theoretical | B. pseudomallei | B. mallei |
| Number | Protein | Number | Function | MW (kDa) | pI | K96243 | ATCC 23344 |
| 1 | DnaK | 83721009 | heat shock | 69.70 | 4.96 | 98% | 98% |
| | | | protein | | | | |
| 2 | HtpG | 83720569 | heat shock | 70.97 | 5.17 | 99% | 99% |
| | | | protein | | | | |
| 3 | 30S ribosomal protein | 83719745 | translation | 62.26 | 5.08 | 100% | 100% |
| 4 | 3-oxoadipate | 83721125 | metabolism | 33.16 | 9.06 | 98% | 98% |
| | CoA-succinyl transferase | | | | | | |
| 5 | AhpC | 83721537 | peroxidase | 23.81 | 5.61 | 98% | 98% |
| 6 | * | | | | | | |
| 7 | * | | | | | | |
| 8 | hypothetical protein | 83717445 | | 11.87 | 5.64 | not present | not present |
| | BTH_11071 | | | | | | |
| 9 | OmpW | 83719376 | outer membrane | 22.71 | 8.6 | 96% | 96% |
| | | | protein | | | | |
| 10 | cpn10 | 83719093 | heat shock | 10.48 | 5.33 | 99% | 99% |
| | | | protein | | | | |
| 11 | CmaB | 83717262 | translation | 35.36 | 5.44 | 97% | not present |
| 12 | ribosomal protein L7 | 83719193 | translation | 12.52 | 4.9 | 97% | 98% |
| 13 | * | | | | | | |
| 14 | * | | | | | | |
| 15 | * | | | | | | |
| 16 | EF-Tu | 83721154 | translation | 42.86 | 5.36 | 100% | 100% |

Three of the proteins, EF-Tu, AhpC, and DnaK, were previously recognized as potential B. pseudomallei antigens using a similar approach with human convalescent sera [Harding S V, Sarkar-Tyson M, Smither S J, Atkins T P, Oyston P C, et al. (2007) The identification of surface proteins of Burkholderia pseudomallei. Vaccine 25: 2664-2672]. Surprisingly, one of the immunogenic proteins identified by both studies was EF-Tu. EF-Tu is best known for its role in bacterial protein synthesis, functioning as a GTPase to catalyze the transfer of aminoacyl-tRNAs to the ribosome [Yokosawa H, Inoue-Yokosawa N, Arai K I, Kawakita M, Kaziro Y (1973) The role of guanosine triphosphate hydrolysis in elongation factor Tu-promoted binding of aminoacyl transfer ribonucleic acid to ribosomes. J Biol Chem 248: 375-377]. However, compelling evidence supports additional functions for EF-Tu, including roles as a bacterial adhesin and invasin for several pathogenic bacteria [Kunert A, Losse J, Gruszin C, Huhn M, Kaendler K, et al. (2007) Immune evasion of the human pathogen Pseudomonas aeruginosa: elongation factor Tuf is a factor H and plasminogen binding protein. J Immunol 179: 2979-2988; Balasubramanian S, Kannan T R, Baseman J B (2008) The surface-exposed carboxyl region of Mycoplasma pneumoniae elongation factor Tu interacts with fibronectin. Infect Immun 76: 3116-3123; Balasubramanian S, Kannan T R, Hart P J, Baseman J B (2009) Amino acid changes in elongation factor Tu of Mycoplasma pneumoniae and Mycoplasma genitalium influence fibronectin binding. Infect Immun 77: 3533-3541; Barel M, Hovanessian A G, Meibom K, Briand J P, Dupuis M, et al. (2008) A novel receptor-ligand pathway for entry of Francisella tularensis in monocyte-like THP-1 cells: interaction between surface nucleolin and bacterial elongation factor Tu. BMC Microbiol 8: 145].

### Example 2

### Burkholderia EF-Tu is Membrane-Associated and Recognized During Natural Infection

Prior work suggests that B. pseudomallei EF-Tu is present on the bacterial surface and is recognized by convalescent sera from human melioidosis patients [Harding S V, Sarkar-Tyson M, Smither S J, Atkins T P, Oyston P C, et al. (2007) The identification of surface proteins of Burkholderia pseudomallei. Vaccine 25: 2664-2672]. Thus, the hypothesis was that EF-Tu may represent a novel immunoprotective antigen. To determine whether EF-Tu is recognized during infection in the murine model of melioidosis, a group of BALB/c mice (N=6) were infected intraperitoneally (i.p.) with 107 cfu of B. thailandensis and harvested sera from survivors two weeks later. The pooled sera from infected mice recognized the recombinant, purified preparation of EF-Tu (rEF-Tu) (FIGS. 2A and B), while sera from uninfected mice did not (not shown). FIG. 2A is a coomassie stained gel of rEF-Tu affinity purified under native conditions by Ni-NTA agarose batch purification (MW=BenchMark Pre-stained molecular weight ladder; Whole-cell lysate (WCL)=5 μg; Flow-through (FT)=5 μg; Washes 1, 3, and 5 with 20 mM imidazole (W1, W2, W3); Elutions 1 and 2 with 250 mM imidazole (E1, E2)=5 μg). FIG. 2B is a Western blot of 10 μg rEF-Tu probed with pooled sera from BALB/c mice infected i.p. with 107 cfu of B. thailandensis (1o Ab, 1:200; 2o Ab 1:1000; MW=BenchMark Pre-stained molecular weight ladder). This indicates that EF-Tu is expressed during infection and is recognized by host antibody in the mouse model. Furthermore, these observations indicate that host antibody generated to native EF-Tu during bacterial infection cross-reacts with rEF-Tu. To determine if rEF-Tu could induce antibodies that recognize native EF-Tu, a group of BALB/c mice (N=6) were immunized subcutaneously with 25 μg rEF-Tu adsorbed to aluminum hydroxide adjuvant and boosted with the same formulation on day 21. On day 35, sera were collected, pooled, and affinity purified for immunoblot of rEF-Tu, as well as whole cell and total membrane protein fractions of B. thailandensis. Pooled sera from rEF-Tu-immunized mice recognized the 47 kDa recombinant form of EF-Tu, as well as native EF-Tu in the whole cell lysate and total membrane fraction (FIG. 2C: Western blot of 0.5 μg rEF-Tu, 15 μg B. thailandensis whole cell lysate (WCL) and 15 μg B. thailandensis total membrane protein (TMP) fractions probed with pooled antisera from rEF-Tu-immunized mice (1o Ab, 1:200; 2o Ab, 1:1000); MW=SeeBlue Plus2 molecular weight ladder). The bands were excised, digested, and analyzed by MALDI-TOF mass spectrometry to confirm their identity. None of the EF-Tu proteins were detected by Western blot using pooled sera from naïve BALB/c mice (not shown). To rule out cytoplasmic EF-Tu contamination in the membrane fraction, a monoclonal antibody against the β subunit of E. coli RNA polymerase (NeoClone) was used to probe E. coli cellular fractions prepared in exactly the same manner as B. thailandensis. A band at 150 kDa corresponding to the β subunit was observed in the whole cell lysate and was absent in the total membrane preparation (FIG. 2D: Western blot of 0.5 μg rEF-Tu, 15 μg E. coli WCL and 15 μg E. coli TMP fractions probed with monoclonal antibody to E. coli β subunit of RNA Polymerase (1o Ab, 1:1000; 2o Ab, 1:1000); MW=SeeBlue Plus2 molecular weight ladder), indicating that the membrane preparation is free of cytoplasmic contamination.

### Example 3

### Burkholderia EF-Tu is Secreted in Outer Membrane Vesicles

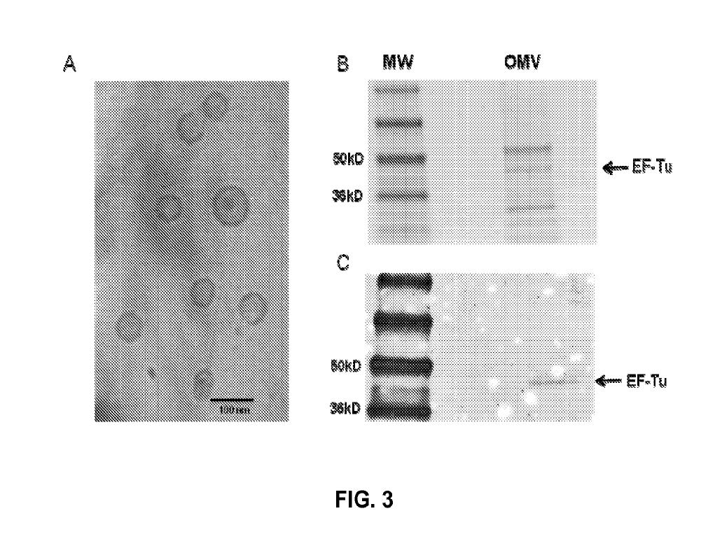

EF-Tu has been demonstrated on the surface of several pathogenic bacteria, including B. pseudomallei and closely-related Pseudomonas aeruginosa [Harding S V, Sarkar-Tyson M, Smither S J, Atkins T P, Oyston P C, et al. (2007) The identification of surface proteins of Burkholderia pseudomallei. Vaccine 25: 2664-2672; Kunert A, Losse J, Gruszin C, Huhn M, Kaendler K, et al. (2007) Immune evasion of the human pathogen Pseudomonas aeruginosa: elongation factor Tuf is a factor H and plasminogen binding protein. J Immunol 179: 2979-2988]. However, attempts to demonstrate EF-Tu on the surface of Burkholderia thailandensis using both immunogold labeling and immunofluorescent microscopy were unsuccessful. EF-Tu lacks a recognizable signal sequence and the mechanism by which EF-Tu is transported to the bacterial surface has remained an enigma. Recent work with bacterial OMVs has demonstrated that OMVs contain numerous virulence factors, including cytoplasmic, periplasmic, and outer membrane constituents [Amano A, Takeuchi H, Furuta N Outer membrane vesicles function as offensive weapons in host-parasite interactions. Microbes Infect]. Therefore, the possibility that EF-Tu, an abundant bacterial protein, might be shed in OMVs was considered. OMVs were prepared from a late logarithmic culture of B. pseudomallei strain 1026b (FIG. 3A: Cryo-transmission electron micrograph of purified OMVs prepared from a late logarithmic culture of B. pseuodomallei strain 1026b, where bar indicates 100 nm; and FIG. 3B: Coomassie-stained gel of OMV preparation (5 μg); MW=SeeBlue plus2 molecular weight ladder) and probed with affinity-purified antibody to EF-Tu. The presence of EF-Tu in B. pseudomallei OMVs was detected (FIG. 3C: Western blot of OMV preparation using affinity purified EF-Tu antibody (1:1000)), which may partially account for the export of EF-Tu from the bacterial cytoplasm.

### Example 4

### Mucosal and Parenteral Immunization with EF-Tu Yields Antigen-Specific IgG and IgA

The ability of rEF-Tu to generate antigen-specific IgG that recognizes the native form of EF-Tu indicates its potential use as a vaccine immunogen. Therefore, a mucosal and parenteral immunization strategy was designed to measure and compare the antibody and CMI responses elicited by rEF-Tu immunization. Groups of BALB/c mice (n=12) were primed either subcutaneously (s.c.) with 25 μg rEF-Tu adsorbed to aluminum hydroxide or intranasally (i.n.) with 25 μg rEF-Tu and 5 μg CpG ODN 1826. CpG ODN is a well-characterized TLR9 ligand that can be administered parenterally or mucosally to drive type 1 immune responses [Freytag L C, Clements J D (2005) Mucosal adjuvants. Vaccine 23: 1804-1813; Klinman D M, Klaschik S, Sato T, Tross D (2009) CpG oligonucleotides as adjuvants for vaccines targeting infectious diseases. Adv Drug Deliv Rev 61: 248-255] and can increase vaccine efficacy against B. pseudomallei [Harland D N, Chu K, Haque A, Nelson M, Walker N J, et al. (2007) Identification of a LolC homologue in Burkholderia pseudomallei, a novel protective antigen for melioidosis. Infect Immun 75: 4173-4180; Chen Y S, Hsiao Y S, Lin H H, Liu Y, Chen Y L (2006) CpG-modified plasmid DNA encoding flagellin improves immunogenicity and provides protection against Burkholderia pseudomallei infection in BALB/c mice. Infect Immun 74: 1699-1705; Elvin S J, Healey G D, Westwood A, Knight S C, Eyles J E, et al. (2006) Protection against heterologous Burkholderia pseudomallei strains by dendritic cell immunization. Infect Immun 74: 1706-1711]. Adjuvant-only (n=5) and naïve mice (n=12) were included as controls. Mice were boosted on day 21 with the same formulations using homologous (s.c.+s.c.; i.n.+i.n.) and heterologous (s.c.+i.n.) prime-boost strategies. Sera and BAL fluid from half (n=6) of the animals in the immunized and naïve groups were harvested on day 35 and assayed for reactivity with rEF-Tu by ELISA.

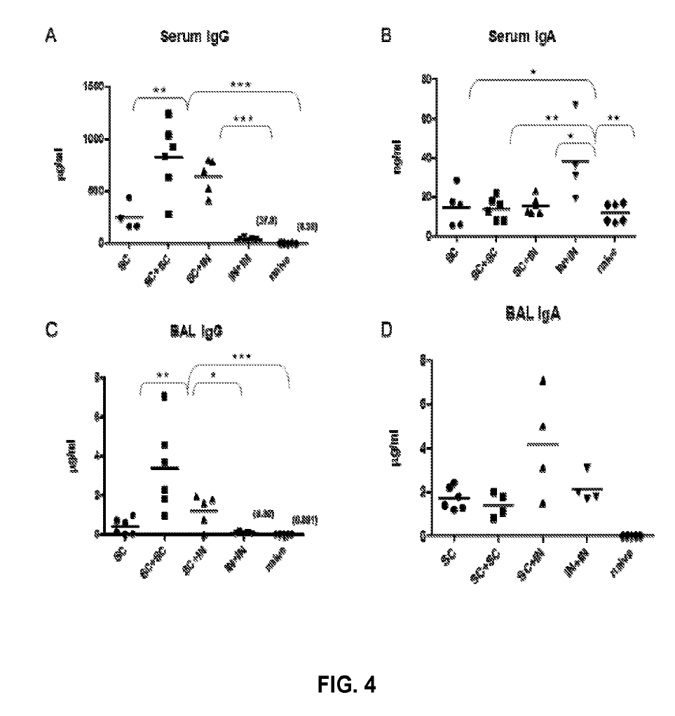

Antigen-specific serum IgG and IgA concentrations were significantly higher in all immunized groups compared to naïve mouse sera (FIGS. 4A and 4B; P<0.001 Serum IgG (A) and IgA (B) measured by ELISA). The s.c.+s.c. mice produced the highest concentrations of EF-Tu-specific serum IgG, while the i.n.+i.n. mice produced the lowest concentrations among the immunized groups. In contrast, induction of EF-Tu-specific serum IgA was only observed in the i.n.+i.n. mice (FIG. 4B). Antigen-specific IgG and IgA in the BAL was significantly higher in all immunized groups compared to BAL from naïve mice (P<0.001). The s.c.+s.c. group produced the greatest concentrations of EF-Tu-specific BAL IgG (FIG. 4C-BAL IgG measured by ELISA). EF-Tu-specific IgA was more than 100-fold higher in the BAL than in the serum of immunized animals regardless of the route of immunization. The median concentration of EF-Tu-specific BAL IgA was highest in the s.c.+i.n. group, although it was not statistically different from the other immunized groups (FIG. 4D-BAL IgA measured by ELISA). Serum IgG (FIG. 4A) and IgA (FIG. 4B) and BAL IgG (FIG. 4C) and IgA (FIG. 4D) were measured by ELISA. SC=subcutaneous immunization with 25 μg rEF-Tu adsorbed 1:1 with aluminum hydroxide adjuvant. IN=intranasal immunization with 25 μg rEF-Tu admixed with 5 μg CpG adjuvant. Horizontal line represents the median value for each group (N=6). Median values are provided in parentheses for IN+IN and naïve groups in panels A and C. (*P<0.05, **P<0.01, ***P<0.001 using the Mann-Whitney test).

In addition, IgG1 and IgG2a in the serum and BAL were assayed to test for any differences in the type 1 and type 2 immune responses elicited in each group. Mice immunized s.c.+s.c. demonstrated IgG1:IgG2a ratios of 5.6 and 140 in the sera and BAL, respectively (TABLE 2).

TABLE 2
Serum and BAL EF-Tu specific IgG1 and IgG2a concentrations (μg/ml)
and ratios

| | Serum | | | BAL | | |
|---|---|---|---|---|---|---|
| Group | IgG1 | IgG2a | ratio | IgG1 | IgG2a | ratio |
| [symbol] C | 124.5 | 13.7 | 9.1 | 0.47 | 0.22 | 22 |
| [symbol] C + SC | 333.7 | 59.6 | 5.6 | 3.7 | 0.02 | 140 |
| [symbol] C + IN | 93.0 | 63.2 | 1.5 | 1.5 | 0.04 | 31 |
| N + IN | 0.28 | 63.3 | 0.004 | 0.07 | 0.009 | 8.4 |

IgG1 and IgG2a were measured by ELISA using sera and BAL from immunized mice (N = 6).
IgG1:IgG2a ratios >1 indicate a type 2 humoral immune response, while ratios <1 indicate a type 1 cellular immune response. [symbol]
indicates data missing or illegible when filed

The predominance of IgG1 is more characteristic of a type 2 immune response [DuBois A B, Freytag L C, Clements J D (2007) Evaluation of combinatorial vaccines against anthrax and plague in a murine model. Vaccine 25: 4747-4754]. Mice immunized s.c.+i.n. and i.n.+i.n. displayed serum IgG1:IgG2a ratios of 1.5 and 0.004, respectively, and demonstrated a shift from IgG1 to IgG2a in the BAL as well (Table 2). These results indicate the generation of a stronger type 1 immune response in the mucosally immunized groups versus those immunized parenterally.

### Example 5

### Th1 and Th2 Cytokine Responses in EF-Tu Restimulated Splenocytes

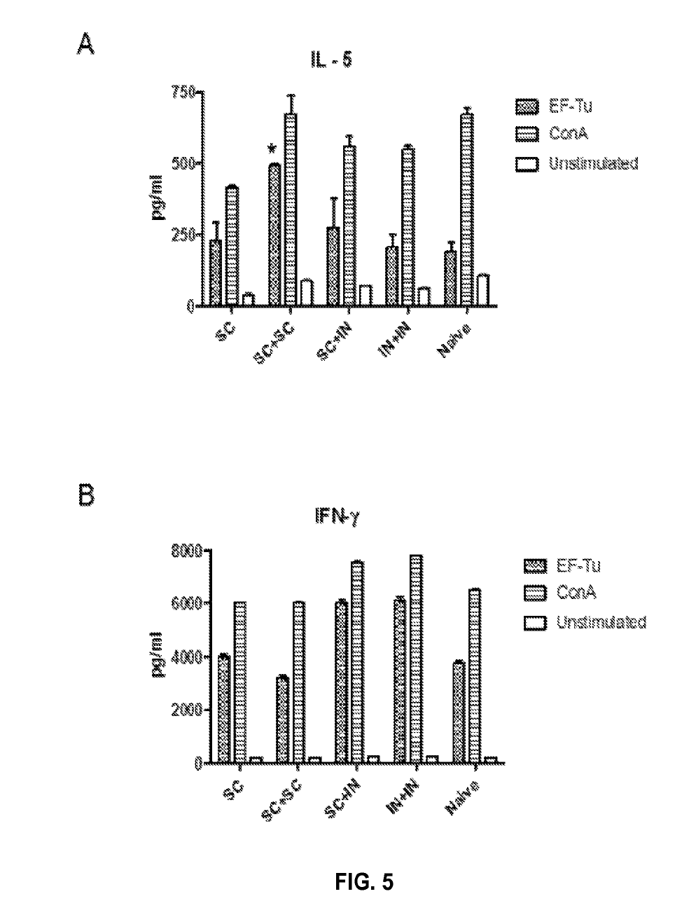

A Th1-driven CMI response, in concert with the production of specific antibodies, is likely essential for vaccine efficacy against B. pseudomallei [Haque A, Chu K, Easton A, Stevens M P, Galyov E E, et al. (2006) A live experimental vaccine against Burkholderia pseudomallei elicits CD4+ T cell-mediated immunity, priming T cells specific for 2 type III secretion system proteins. J Infect Dis 194: 1241-1248; Healey G D, Elvin S J, Morton M, Williamson E D (2005) Humoral and cell-mediated adaptive immune responses are required for protection against Burkholderia pseudomallei challenge and bacterial clearance postinfection. Infect Immun 73: 5945-5951]. To assess antigen-specific T cell responses in rEF-Tu immunized mice, spleens were harvested on day 35 (2 weeks post-immunization) and restimulated in vitro with rEF-Tu. Cell culture supernatants were assayed on day three for IFN-γ and IL-5 production as an indication of Th1 and Th2 responses, respectively. Mice that were immunized s.c+s.c. produced significantly higher levels of IL-5 compared to naïve animals (FIG. 5A; P<0.05) upon restimulation with rEF-Tu. In contrast, mice that received one dose of rEF-Tu s.c. and both mouse groups boosted mucosally (s.c.+i.n. and i.n.+i.n.) produced similar levels of IL-5 compared to naïve mice (FIG. 5A). Both groups that were boosted mucosally (s.c.+i.n. and i.n.+i.n.) produced higher levels of IFN-γ than mice that were immunized parenterally (s.c. only and s.c.+s.c.) and naïve mice (FIG. 5B), although this increase was not statistically significant. For the data of FIGS. 5A and B, splenocytes from individual mice in each treatment group (N=6) were restimulated in triplicate with rEF-Tu (1 μg) or ConA (1 μg) or left unstimulated, and cell culture supernatants were assayed in duplicate on day 3 for IL-5 (FIG. 5A) and IFN-γ (FIG. 5B) cytokine production using a multiplex assay. Error bars represent the standard error of the mean (SEM) for each group (*P<0.05 using a two-way ANOVA).

### Example 6

### Mucosal Immunization with EF-Tu Reduces Bacterial Burden in the Lung

EF-Tu-immunized mice were challenged with B. thailandensis as a preliminary measure of protective capacity in an in vivo test system. B. thailandensis is not considered a human pathogen, however it is lethal in inbred mouse strains (BALB/c and C57Bl/6) at aerosol challenge doses of 1×105 cfu or higher [West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17]. Therefore mice (N=5-6) were challenged in immunized, adjuvant-only, and naïve groups with 5×105 cfu (˜LD50) of B. thailandensis by aerosol on day 35. All mice were sacrificed 24 hr later to assess lung bacterial burdens since there is a direct correlation between lung bacterial burden and disease progression in this acute pneumonia model [West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126; Morici L A, Heang J, Tate T, Didier P J, Roy C J Differential susceptibility of inbred mouse strains to Burkholderia thailandensis aerosol infection. Microb Pathog 48: 9-17]. Mice that were primed s.c. and boosted either s.c. or i.n. (s.c.+s.c., s.c.+i.n.) had similar numbers of bacteria in the lungs compared to control mice (FIG. 6). Significantly lower bacterial burdens in lung tissues were observed in the i.n.+i.n. mice when compared to the adjuvant only (CpG) and naïve groups (P<0.05; FIG. 6). For FIG. 6, lung bacterial burdens (cfu/g tissue) were determined in naïve (N=6), adjuvant-only (N=5), and immunized (N=6) mice 24 hrs post-aerosol challenge with 5×105 cfu (˜LD50) of B. thailandensis. SC=subcutaneously immunized; IN=intranasally immunized. Horizontal line represents the geometric mean for each group. (*P<0.05 using the Mann-Whitney test). Nevertheless, BALB/c mice immunized with EF-Tu/CpG (n=4) were not protected from lethal aerosol challenge with B. pseudomallei (500 cfu), as shown by FIG. 9.

### Example 7

### Subcutaneous and Mucosal Immunizations with B. Pseudomallei OMVs Induce Robust IgG Response

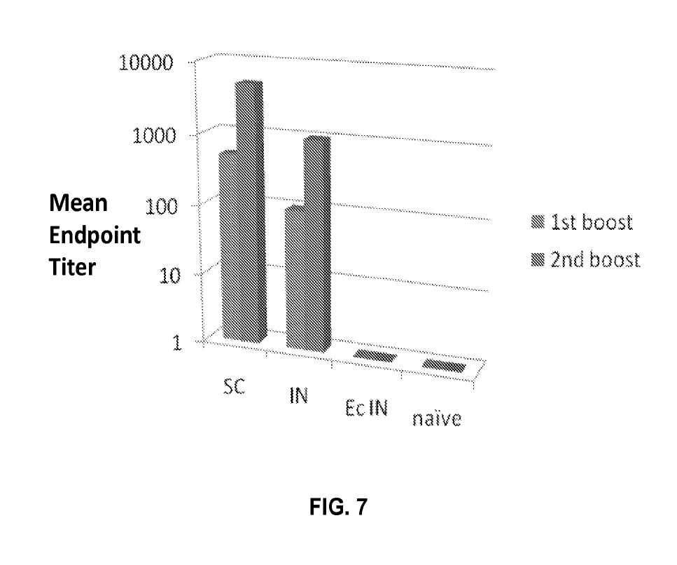

Mice were immunized subcutaneously (SC) or intranasally (IN) with 2.5 μg of purified B. pseudomallei (Bp) OMVs or 2.5 g of E. coli OMV, administered intranasally—“Ec IN” on days 0, 21 (first boost), and 42 (second boost). Naïve mice were not treated. Prior to intranasal immunization, mice were anesthetized via the i.p. route with 0.88 mg/kg ketamine/xylazine in saline in a final volume of 100 μL. As shown in FIG. 7, mice immunized with Bp OMVs either subcutaneously or intranasally demonstrated robust Bp OMV-specific IgG responses after the first boost (left-hand columns) that increased approximately 1 log after the second boost (right-hand columns). In contrast, naïve mice and mice immunized with Ec OMVs did not produce any detectable IgG that recognized Bp OMVs. This demonstrates that antibody production to the Bp OMVs is highly specific and does not appear to cross-react with OMVs from other Gram-negative bacteria such as E. coli.

In order to determine if the OMVs could elicit protection, mice were immunized subcutaneously with Bp OMV or sham (saline only) and were challenged on day 70 with a lethal dose of B. pseudomallei (500 cfu) by aerosol, and survival was monitored for 14 days. One hundred percent (100%) of the sham-immunized mice (n=7) succumbed to challenge within 4 days, while—unexpectedly—80% of the OMV-immunized mice survived until the study endpoint and appeared to have completely recovered as determined by normal behavior/activity and confirmed absence of bacteria in the lungs (FIG. 10).

Thus, OMVs prepared from at least one Burkholderia spp. represent a useful immunogen that may confer protection against Burkholderia infections, and immunization with these OMVs represents a useful method for preventing and possibly preventing Burkholderia infections in animals (including humans).

### Example 8

Burkholderia pseudomallei, and other members of the Burkholderia, are among the most antibiotic resistant bacterial species encountered in human infection. Mortality rates associated with severe B. pseudomallei infection approach 50% despite therapeutic treatment. A protective vaccine against B. pseudomallei would dramatically reduce morbidity and mortality in endemic areas and provide a safeguard for the U.S. and other countries against biological attack with this organism.

This exemplary study investigates the immunogenicity and protective efficacy of B. pseudomallei-derived outer membrane vesicles (OMVs). Vesicles are produced by Gram-negative and Gram-positive bacteria and contain many of the bacterial products recognized by the host immune system during infection. This exemplary study demonstrates that subcutaneous (SC) immunization with OMVs provides significant protection against an otherwise lethal B. pseudomallei aerosol challenge in BALB/c mice. Mice immunized with B. pseudomallei OMVs displayed OMV-specific serum antibody and T-cell memory responses. Furthermore, OMV-mediated immunity appears species-specific as cross-reactive antibody and T cells were not generated in mice immunized with Escherichia coli-derived OMVs. These results provide the first compelling evidence that OMVs represent a non-living vaccine formulation that is able to produce protective humoral and cellular immunity against an aerosolized intracellular bacterium. This vaccine platform constitutes a safe and inexpensive immunization strategy against B. pseudomallei that can be exploited for other intracellular respiratory pathogens, including other Burkholderia and bacteria capable of establishing persistent infection.

### Introduction

The genus Burkholderia encompasses a large group of ubiquitous Gram-negative bacteria pathogenic for both plants and animals. Members of the Burkholderia responsible for human disease include the opportunistic Burkholderia cepacia complex (Bcc), including B. cenocepacia and B. multivorans, which have emerged as significant causes of fatal pulmonary infection in individuals with cystic fibrosis in the United States, Canada, and Europe [1]. Burkholderia mallei, the etiologic agent of glanders, is an obligate mammalian pathogen that primarily infects hoofed animals, but severe human cases have been documented [2]. Lastly, the facultative intracellular bacterium, B. pseudomallei, is the causative agent of melioidosis, an emerging disease responsible for significant morbidity and mortality in Southeast Asia and Northern Australia [3,4]. While most reported cases of B. pseudomallei infection are restricted to these geographic regions, the organism has a much larger global distribution and human cases are likely under-reported [5]. Natural infection with the Burkholderia can occur through subcutaneous inoculation, ingestion, or inhalation of the bacteria. Clinical manifestations can be non-specific, widely variable, and often depend upon the route of inoculation and the immune status of the host [3]. Burkholderia infections are inherently difficult to treat due to their resistance to multiple antibiotics, biofilm formation, and establishment of intracellular and chronic infection in the host. Preventive measures such as active immunization could dramatically reduce the global incidence of disease; however there is currently no commercially available vaccine against any member of the Burkholderia [6].

In recent years, a number of vaccine strategies against B. pseudomallei and B. mallei have been explored due to the potential threat of these organisms as biological warfare agents. No ideal candidate has yet emerged from pre-clinical studies [7]. For B. pseudomallei, inactivated whole-cell preparations and live-attenuated strains are highly immunogenic and demonstrate partial to full protection in murine models [7-10]. However, safety concerns and contraindication for use in immunocompromised individuals limits the utility of such vaccines for human use. Safer, alternative approaches to vaccination include use of purified preparations of lipopolysaccharide (LPS), capsular polysaccharide (CPS), or protein-based subunit vaccines. Studies with B. pseudomallei LPS and CPS have demonstrated high degrees of antibody-mediated short-term protection with both active and passive immunization [11-14]. However, the inability of these T-cell independent antigens to confer sterilizing immunity is problematic. Polysaccharide-protein conjugate vaccines that promote T-cell-dependent immune responses may improve efficacy, but the high cost and technical expertise associated with such vaccines may explain the current absence of active immunization studies in the literature [7]. Protein subunit strategies have yielded variable degrees of protection against systemic B. pseudomallei infection but have proved either ineffective or have not been tested against inhalational challenge [15-18]. Pulmonary infection with B. pseudomallei is highly lethal in humans and animal models and has been particularly difficult to prevent by vaccination thus far [7,19]. A successful vaccine against B. pseudomallei, as with other intracellular bacteria, will likely require the induction of both humoral and cellular-mediated immune (CMI) responses for complete protection and eradication of persistent bacteria [20]. Furthermore, the vaccine must be safe and efficacious against multiple routes of infection.

Reported herein is an immunization approach against B. pseudomallei that utilizes bacteria-derived outer membrane vesicles (OMVs). OMVs are constitutively produced by Gram-negative bacteria both in vivo and in vitro and are often enriched in virulence factors and Toll-like receptor (TLR) agonists [2]-23]. Vesicle production has also been observed in fungi and Gram-positive bacteria highlighting the conservation of this process among microbes, although the mechanisms of secretion likely differ [21]. Use of membrane vesicle-based vaccines is rapidly gaining interest, and vesicle-mediated protection against mucosal and systemic bacterial challenge has been demonstrated for Neisseria meningitides [24], Bordetella pertussis [25], Salmonella typhimurium [26], Vibrio cholerae [27], and more recently Bacillus anthracis [28]. In mouse studies, efficacy of vesicle vaccines has ranged from 33% protection against B. anthracis [28] to nearly 100% protection against V. cholerae [27]. N. meningitidis serogroup B OMVs adsorbed to aluminum adjuvant are approved for human use and provide 80% protective efficacy against severe invasive disease [24]. In this instance, protection is mediated by serum bactericidal antibody directed against Neisseria surface antigens thus promoting bacterial opsonization and complement-mediated killing [29].

This exemplary study demonstrates that immunization with naturally shed B. pseudomallei OMVs provides significant protection against lethal aerosol challenge in a murine model of melioidosis. Membrane vesicles represent an efficacious vaccine platform against other aerosolized intracellular pathogens, including those that establish persistent infection.

### Bacterial Strains and Culture

B. pseudomallei strain 1026b was obtained from BEI Resources. Escherichia coli strain M15 was obtained from Qiagen. Bacteria were cultured from glycerol stocks immediately prior to use and single colonies were selected from freshly streaked LB agar plates. Overnight cultures were diluted 1:100 in fresh LB and incubated with shaking at 37° C. until OD600 reached 0.75 for challenge experiments.

### Outer Membrane Vesicle (OMV) Preparation and Characterization

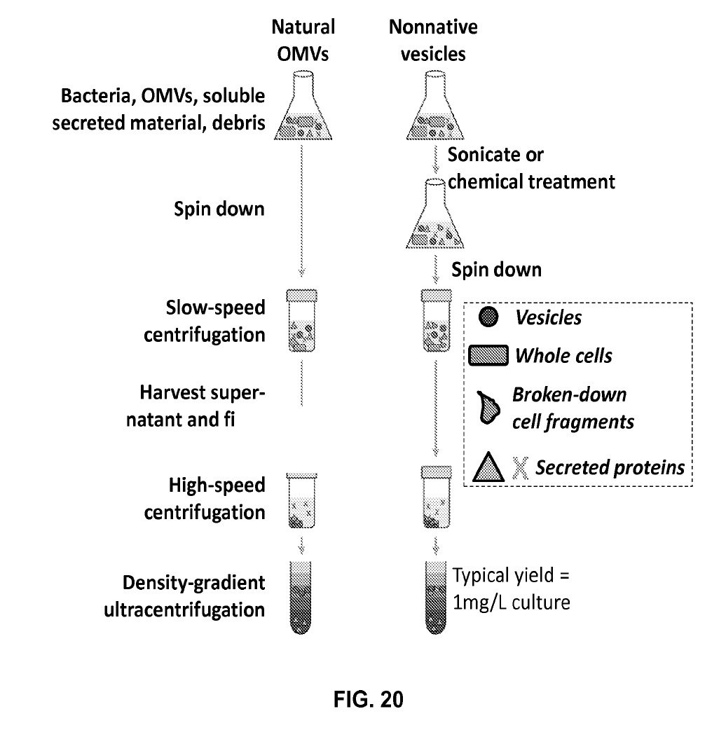

OMVs were purified as previously described for example in Nieves, W. et al., PLoS One, 5(12):314361 (2010), the disclosure of which is incorporated herein by reference. An exemplary procedure for preparing and purifying OMVs according to the invention is illustrated in FIG. 20 and described, for example, in Kulp, A. et al., Annu. Rev. Microbiol., 64: 163-184 (2010), the disclosure of which is incorporated herein by reference. Pooled OMVs were desalted and concentrated using a 100 kDa Amicon desalting column (Millipore) following the manufacturer's protocol. OMVs were then washed and resuspended in LPS-free water. OMVs were quantified with a Bradford Protein Assay (Bio-Rad). Cryo Transmission Electron Microscopy was performed using a JEOL 2010 transmission electron microscope to visually confirm the presence and purity of OMVs. For LC-MS analysis, 100 g of OMVs were separated by SDS-PAGE and the gel bands were manually cut into pieces and rinsed twice with 25 mM ammonium bicarbonate in 50% acentonitrile for 20 min. Proteins were digested with trypsin (1 μg per band) in 25 mM ammonium bicarbonate at 37° C. overnight (16 h). The peptides were extracted by adding 100 μl of extraction buffer (0.1% formic acid in 50% acentonitrile aqueous solution), incubating for 20 min, and collecting the supernatant. This step was repeated once, followed by incubation in 100% acetonitrile. The combined supernatants were dried down in an Eppendorf Vacufuge. Prior to LC-MS analysis, the peptides were resuspended in 10 (1 of 0.1% formic acid/2% acetonitrile. All spectra were acquired on a Thermo-Fisher LTQ-XL linear ion trap mass spectrometer (Waltham, Mass.) coupling with an Eksigent nanoLC 2D (Dublin, Calif.). Peptides were loaded into a Dionex PepMap C18 trap column (300 μm internal diameter×5 mm, 5 μm particle size) and then separated by a New Objective reversed phase C18 Picofrit column/emitter (75 (m id, 10 cm long, 5 (m particle size, Woburn, N.J.). A gradient elution at 250 nl/min starting from 5% to 40% buffer B in 40 min, followed by 40-80% buffer B in 20 min, then 80% buffer B for 10 min. Buffer A is 0.1% formic acid aqueous solution and Buffer B is 0.1% formic acid in acetonitrile. A blank run was inserted between two sample runs to reduce cross contamination. The raw data were searched against Burkholderia pseudomallei K96243 proteome (2009-12-06) downloaded from the Burkholderia Genome Database (http://www.Burkholderia.com). The search engine Bioworks 3.3.1 (Thermo-Fisher) was used with Protein-Prophet and Trans Proteomic Pipeline, as described for example, in Keller A. et al., Mol Syst Biol, 1:0017 (2005) and Keller A. et al., Anal Chem, 74(20): 5383-92 (2002), the disclosures of each of which are hereby incorporated by reference. Protein matches are reported with an error rate of 2.5% predicted by ProteinProphet as the threshold.

### LPS and CPS Determination

The amount of LPS in B. pseudomallei OMVs was determined by capture ELISA. Maxisorp immunoplates (Nunc) were coated overnight at 4° C. with 100 μl of 5 μg/ml of anti-B. pseudomallei LPS monoclonal antibody (Mab) (from J. Prior and S, Ngugi, Dsd, UK) in PBS. After washing with PBS/0.05% Tween 20 (PBST), plates were blocked with 3% skimmed milk in PBS. Plates were then incubated for 1 h at 25° C. with 1:2 dilutions of OMVs or purified B. thailandensis LPS, starting at 400 μg/ml, in 3% milk/PBS/0.05% Tween/0.8% polyvinylpyrrolidone (PVP). The anti-B. pseudomallei LPS Mab was biotinylated using the EZ-link micro sulfo-NHS-LCbiotinylation kit (Thermo-Pierce), following the manufacturer's recommended protocol. Biotinylated anti-B. pseudomallei LPS Mab in 3% milk/PBS/0.05% tween/0.8% PVP was added to plates at a concentration of 1 μg/ml and incubated for 1 h. Plates were washed in PBS/T and then incubated for 1 h at 25° C. with a streptavidinperoxidase polymer conjugate (Sigma), diluted 1:1000 in 3% milk/PBS/0.05% tween/0.8% PVP. Plates were then washed prior to development with 1-step Ultra TMB ELISA reagent (Thermo Scientific). Plates were read at 450 nm after the addition of 2 M H2SO4 to stop the reaction. A standard curve of A450 vs. LPS concentration was plotted and used to determine the LPS content of OMV samples.

The presence of CPS in OMVs was determined by Western blot using monoclonal antibody 3C5, specific for B. pseudomallei CPS as described, for example, in Nuti D. et al., mBio, 2(4), e00136-11 (2011), the disclosure of which is incorporated herein by reference. Ten μg of OMVs, B. pseudomallei 1026b lysate, and B. thailandensis lysate were separated by sodium dodecyl sulphate polyacrylamide gel electrophoresis (SDS-PAGE) using a 7.5% polyacrylamide gel (Bio-Rad). The proteins were transferred to a nitrocellulose membrane and blocked in 1.5% BSA in TBS-T for 1 h. The membrane was incubated with 3C5 IgG3 (1:1000 dilution) overnight at 4° C., washed 3 times with TBS-T, and incubated with goat anti-mouse HRP-conjugated secondary antibody (Pierce, 1:1000 dilution) for 1 h at room temperature. The membrane was washed and developed using Opti-4CN substrate (BioRad).

### Animals

Female BALB/c mice 8- to 10-weeks-old were purchased from Charles River Laboratories (Wilmington, Mass.) and maintained 5 per cage in polystyrene microisolator units under pathogen-free conditions. Animals were fed sterile rodent chow and water ad libitum and allowed to acclimate 1 week prior to use. This study was carried out in strict accordance with the recommendations in the Guide for the Care and Use of Laboratory Animals of the National Institutes of Health (NIH). The protocols were approved by Tulane University Health Sciences Center and Tulane National Primate Research Center Institutional Animal Care and Use Committees.

### Immunizations

Two independent immunization experiments were performed using separately prepared batches of purified OMV. In the first experiment, BALB/c mice (n=10 per group) were primed subcutaneously (SC) on day 0 with 2.5 μg of B. pseudomallei OMVs in a final volume of 100 μl sterile saline, or intranasally (IN) with 2.5 μg B. pseudomallei or E. coli OMVs in a final volume of 7.5 μl/nostril. Prior to IN immunization, mice were briefly anesthetized with Isoflurane (VetOne). Naïve mice did not receive any treatment. Immunized mice were boosted on days 21 and 42 with the same formulations. No adjuvant was added to the OMV preparations. One month after the last immunization, a subset of mice (n=5 per group) was utilized for measurement of antibody responses and separate groups of mice (n=5 mice group) were challenged with B. pseudomallei by aerosol. In the second experiment, BALB/c mice (n=15 per group) were immunized exactly as described above. Five mice per group were utilized to determine immune correlates of protection, and ten mice per group were challenged with B. pseudomallei by aerosol.

### Aerosol Challenges

Mice were challenged with B. pseudomallei strain 1026b via small particle aerosol as previously described, for example, at Morici L. A. et al., Microb Pathog, 48(1): 9-17 (2010), the disclosure of which is incorporated herein by reference. Animal groups were randomized for experimental infection; the animal capacity for each discrete run of the inhalation system was 23; the total number of runs required was three. A dynamic nose-only inhalation exposure system (CH Technologies, Westwood, N.J.) was employed for the exposures. The inhalation apparatus was housed in a Class III biological safety cabinet (GermFree Laboratories, Ormond Beach, Fla.) within a BSL-3 containment laboratory environment. The nose-only system was maintained at 11 lpm total flow during exposures. The aerosols were generated into the central plenum of the chamber using a three-jet collison nebulizer (BGI Inc., Waltham, Mass.). The experimental atmosphere was continuously sampled using an all glass impinger (AGI-4, Ace Glass, Vineland, N.J.) inserted into one of the nose-only ports of the exposure plenum. The impinger contents were cultured immediately after each discrete run of the system and the bacterial colony counts were used to calculate an aerosol concentration (Ca) of B. pseudomallei within the plenum of the nose-only exposure apparatus. The resultant Ca for each run was applied to a calculated breathing rate of the mice to attain a total respiratory volume during exposure. The resulting inhaled dose was expressed in CFU/animal. The mean inhaled dose across all experimental groups was 5.35×103±3.64×103 CFU. Mice were challenged with a target dose of 5LD50 (˜1000 CFU for B. pseudomallei 1026b as determined in pilot experiments). Two naïve mice were included in each exposure run and were euthanized immediately after challenge. Lungs were plated for determination of bacterial CFU to confirm the inoculum.

### CFU Recovery

Lung, spleen, and liver tissue homogenates were used to determine bacterial burden at 14 and 30 days post-infection in mice that survived aerosol challenge. Tissues were aseptically removed, weighed, and individually placed in 1 ml 0.9% NaCl and homogenized with sterile, disposable tissue grinders (Fisher Scientific). Ten-fold serial dilutions of lung homogenates were plated on Pseudomonas isolation agar (PIA). Colonies were counted after incubation for 3 days at 37° C. and reported as CFU per organ.

### Analysis of Antibody Response

Immunized and naïve mice were anesthetized and blood was collected by retro-orbital bleed prior to each immunization. One month after the last immunization, blood samples from immunized and naive mice were collected following euthanasia for determination of antigen-specific serum antibody concentrations. Blood was allowed to clot for 30 min at room temperature and then centrifuged at 2300×g; serum was collected and stored at −80° C. until assayed. The concentrations of serum OMV-specific total IgG, IgG1, IgG2a, and IgA were analysed by enzyme-linked immunosorbent assay (ELISA). Ninety-six-well microtiter plates were coated with 0.5 μg per well of purified B. pseudomallei OMVs in coating buffer (0.1 M sodium bicarbonate, 0.2 M sodium carbonate) and incubated overnight at 4° C. The plates were washed three times with PBS containing 0.05% Tween-20 (PBST). For measurement of IgA, plates were additionally blocked with 2% BSA for 1 h followed by three washes with PBST. All plates were incubated with twofold serial dilutions of sera samples for 2 h at room temperature. Plates were washed three times with PBST and then incubated with either alkaline phosphatase (AP)-conjugated rat anti-mouse IgG, IgG1, IgG2a (1:300 dilution in PBST) (BD Pharmingen) or APconjugated goat-anti-mouse IgA (1:2000) (Invitrogen) for 1 h at room temperature. At the end of the incubation, the plates were washed three times with PBST and developed with SIGMAFAST p-nitrophenyl phosphate tablets (Sigma, St. Louis, Mo.) dissolved in diethanolamine buffer (1 mg/ml). After 15-30 min of incubation, reaction solutions were stopped with 2 M NaOH and read at 405 nm using a μQuant microplate reader and analysed with Gen5 software (BioTek, Winooski, Vt.). The results obtained are expressed as the mean reciprocal endpoint titers for total IgG; concentrations for IgG and IgA; and ratios of IgG1 to IgG2a based upon total concentrations. Endpoint titer is defined as the greatest dilution that yielded an optical density (OD450) greater than three standard deviations above the mean OD450 for pre-immune titers. Concentrations were determined by comparison to a standard curve as previously described, for example, in Nieves W. et al., PLoS One, 5(12):e14361 (2010), the disclosure of which is incorporated herein by reference.

### Antigen Restimulation Assay

Restimulation assays were performed with splenocytes from immunized and naïve mice for analysis of T cell responses. Spleens were removed aseptically and single-cell splenocyte suspensions from each mouse were obtained by passing the spleens through sterile 40 μm cell strainers (Fisher Scientific). Cells were washed twice with Hank's buffered saline solution (HBSS) (ATCC). Cell pellets were resuspended in HBSS and layered onto ACK Lysing buffer (Gibco) for 4 min. Splenic mononuclear leukocyte isolation was achieved by centrifugation at 1500×g for 10 min. Leukocytes were recovered at the interface, washed twice with HBSS, and resuspended in Advanced RPMI 1640 medium (ATCC) supplemented with 10% FBS (Atlanta Biologicals) and 1% antibiotic-antimycotic (Gibco). Cells were plated in a 96-well microtiter plate at 1.5×106 cells/well. Cell cultures were stimulated with 2 μg of B. pseudomallei OMVs, 1 μg ConA (Sigma), or left unstimulated as negative controls. The cultures were incubated at 37° C. in 5% CO2, and cell culture supernatants from each treatment group were collected after 72 h and stored at −80° until use.

### Statistical Analyses

All analyses were performed using GraphPad Prism version 5.0 (GraphPad Software, Inc., La Jolla, Calif.). Statistical analyses were performed using a one-way or two-way ANOVA with Bonferroni's post-test. Values of P<0.05 were considered statistically significant. For survival analysis, the log rank Mantel-Cox test was used.

### Results

(1) B. pseudomallei OMVs contain LPS, CPS, and Protein Antigens

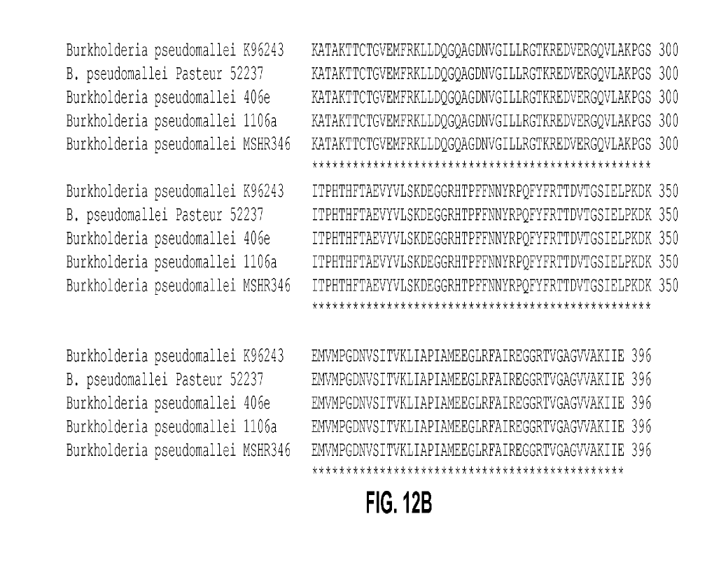

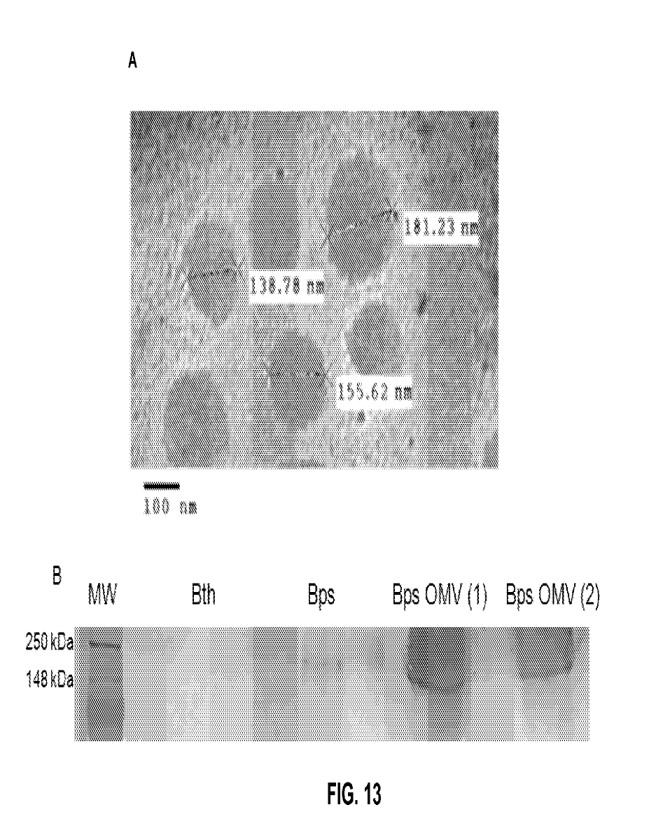

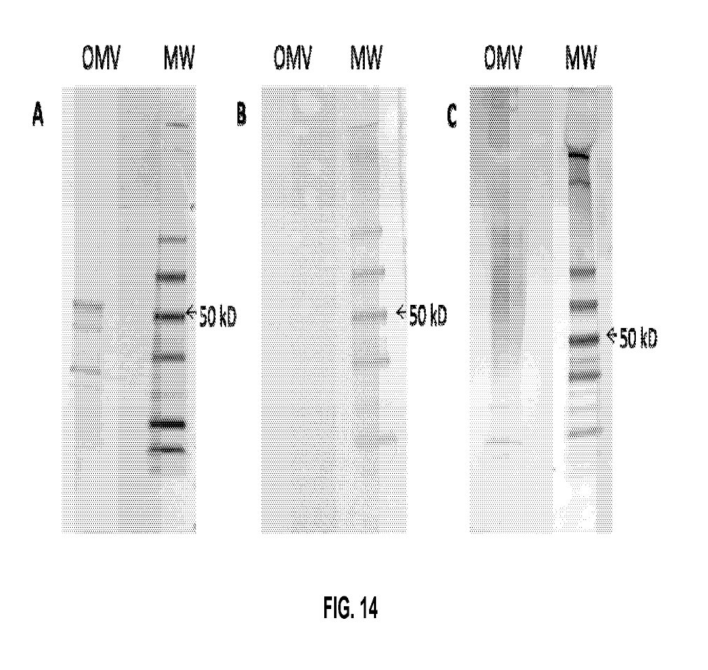

It was previously shown that OMVs are abundantly shed by broth-grown B. pseudomallei and can be harvested from culture supernatants using density gradient ultra-centrifugation, as described, for example, in Nieves W. et al. (2010). Purified B. pseudomallei vesicles range in size from 50 to 250 nm and contain between 1 and 1.5 mg protein per liter of culture (FIG. 13). Using LC-MS analysis, numerous proteins in the OMVs were detected, including 17 putative periplasmic proteins and 12 predicted outer membrane or extracellular proteins (Table 3). Several of the proteins identified were previously characterized immunogenic proteins (Table 3; proteins highlighted in bold). See, for example, Nieves W. et al. (2010) and Harding, S. V. et al., Vaccine, 25(14):2664-72 (2007), the disclosures of which are incorporated herein by reference. While not wishing to be bound by any particular theory, it appears that OMVs shed by broth-grown B. pseudomallei possess similar antigenic cargo as those expressed during infection in vivo. This effect was demonstrated in the present study, which utilized convalescent sera from a rhesus macaque that had been recovered from experimental B. pseudomallei aerosol infection. As shown in FIG. 14, broth-grown bacteria produce OMVs that contain numerous immunoreactive antigens expressed and recognized during B. pseudomallei infection in a non-human primate model of melioidosis. Due to the nature of OMV biogenesis, this study further investigated the ability of OMVs to harbor LPS and CPS, both of which stimulate protective antibody responses against B. pseudomallei. See, for example, Jones S. M. et al., J Med Microbiol, 51(12):1055-62 (2002); Nelson, M. et al., J Med Microbiol, 53(12): 1177-82 (2004); Ngugi, S. A. et al., Vaccine, 28(47):7551-5 (2010), the disclosures of each of which are incorporated herein by reference. Limulus assay confirmed the presence of LPS in the OMVs; OMVs contained 200 μg/ml of LPS as determined by capture ELISA. Using a monoclonal antibody directed against the CPS of B. pseudomallei, as described, for example, in Nuti, D. et al., mBio, 2(4), e00136-11 (2011), this study demonstrated by Western blot that this surface antigen is also abundant in the OMVs (FIG. 13B). The presence of numerous immunoreactive proteins, as well as LPS and CPS, in the B. pseudomallei OMVs is a beneficial property that can be utilized in a potential vaccine.

(2) B. Pseudomallei OMVs Induce Specific Antibody Responses without a Requirement for Adjuvant

OMV biogenesis generates vesicles that contain large quantities of LPS with inherent endotoxicity. Thus, vaccine preparations utilizing OMVs from Gram-negative bacteria will most often require LPS extraction or de-toxification of lipid A prior to administration. See, for example, Koeberling O. et al., J Infect Dis., 198(2):262-70 (2008) and van de Waterbeemd B. et al., Vaccine, 28(30):4810-6 (2010), the disclosures of each of which are incorporated herein by reference. Furthermore, the removal of LPS from OMVs often necessitates the addition of adjuvant to restore OMV immunogenicity. B. pseudomallei LPS is up to 1000-fold less toxic than E. coli LPS, as described, for example, in Utaisincharoen P. et al., Clin Exp Immunol, 122(3): 324-9 (2000) and Matsuura M. et al., FEMS Microbiol Lett, 137(1):79-83 (1996), the disclosures of each of which are incorporated herein by reference. No cytotoxicity was observed in this study in murine macrophages co-cultured with 5 μg of B. pseudomallei OMVs for 72 h. This study exploited the natural adjuvanticity and low toxicity of B. pseudomallei LPS as a native component of the OMV preparation.

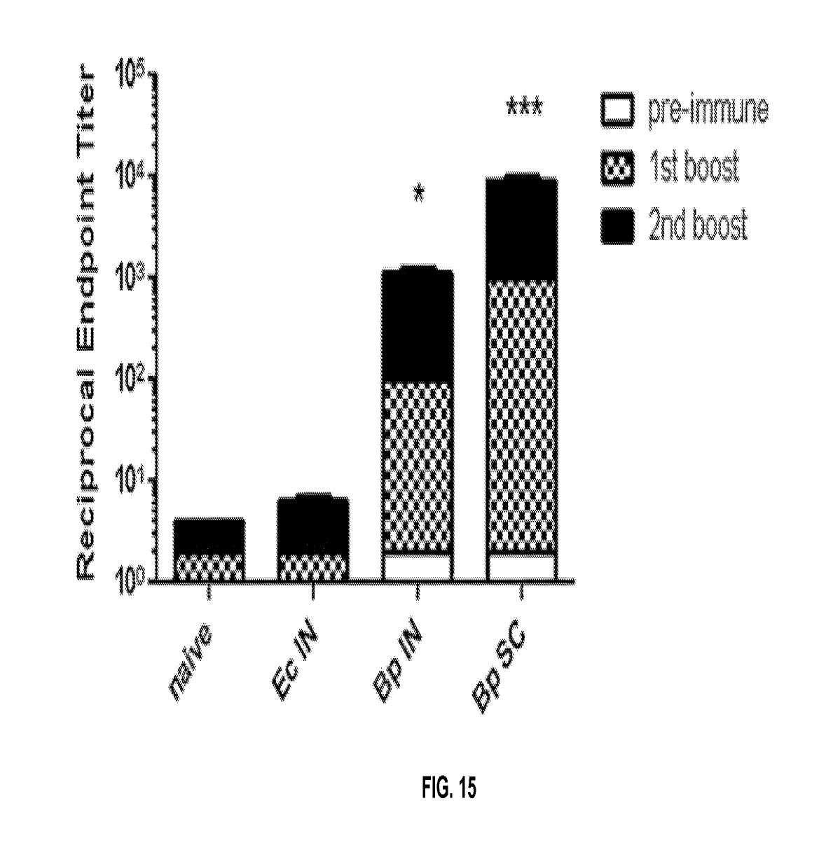

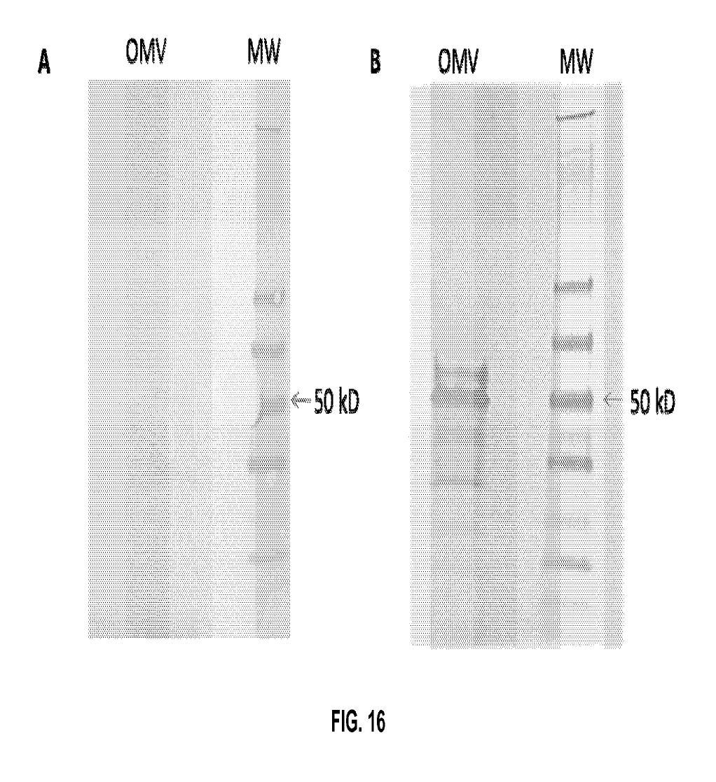

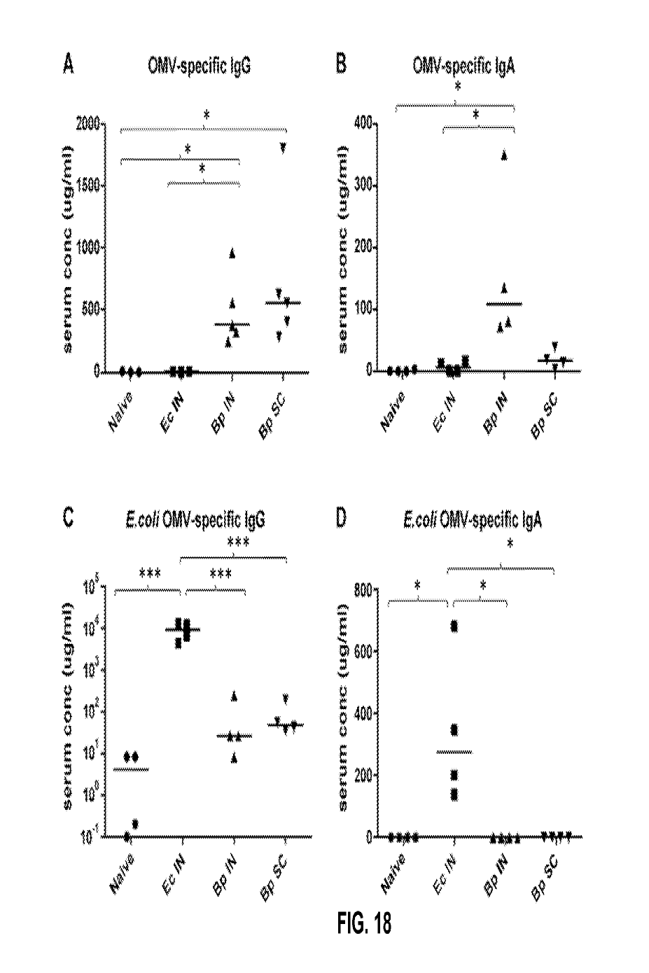

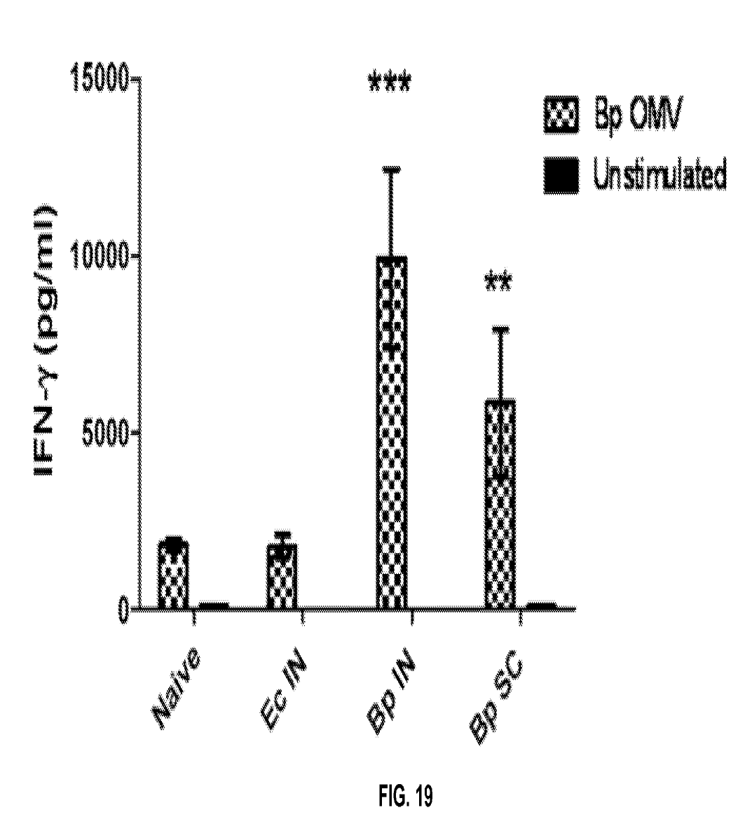

Two groups of mice were immunized with 2.5 μg of B. pseudomallei OMVs by the intranasal (IN) or SC route and boosted on days 21 and 42. In order to examine specificity of the antibody response to the OMVs, we also purified OMVs from a non-pathogenic strain of E. coli as a control antigen. The E. coli OMVs were prepared in exactly the same manner as the B. pseudomallei OMVs and contained LPS. For this reason, mice were immunized with E. coli OMVs by the IN route only due to significant endotoxicity associated with E. coli LPS administered SC, as described, for example, in Schaedler R. W. et al., J Exp Med, 113:559-70 (1961), the disclosure of which is incorporated herein by reference. No additional adjuvant was added to either OMV preparation. B. pseudomallei OMVs administered SC or IN induced high titers of OMV-specific serum IgG after a single boost. Moreover, serum IgG titers increased approximately 1-log after a second boost and were significantly higher than pre-immune titers (FIG. 15). OMV immunization generated IgG responses against multiple protein antigens in the OMV preparation (FIG. 16). Furthermore, the IgG response to B. pseudomallei OMVs appears specific since mice immunized with E. coli OMVs did not generate IgG that recognized B. pseudomallei OMVs. This was not due to immune tolerance because E. coli OMV-immunized mice produced antibodies that recognized their cognate OMVs (FIGS. 18C and 18D). Naïve mice also did not possess antibody that recognized B. pseudomallei OMV antigens (FIG. 15 and FIG. 16).

(3) Immunization with B. Pseudomallei OMVs Provides Significant Protection Against Lethal Aerosol Challenge

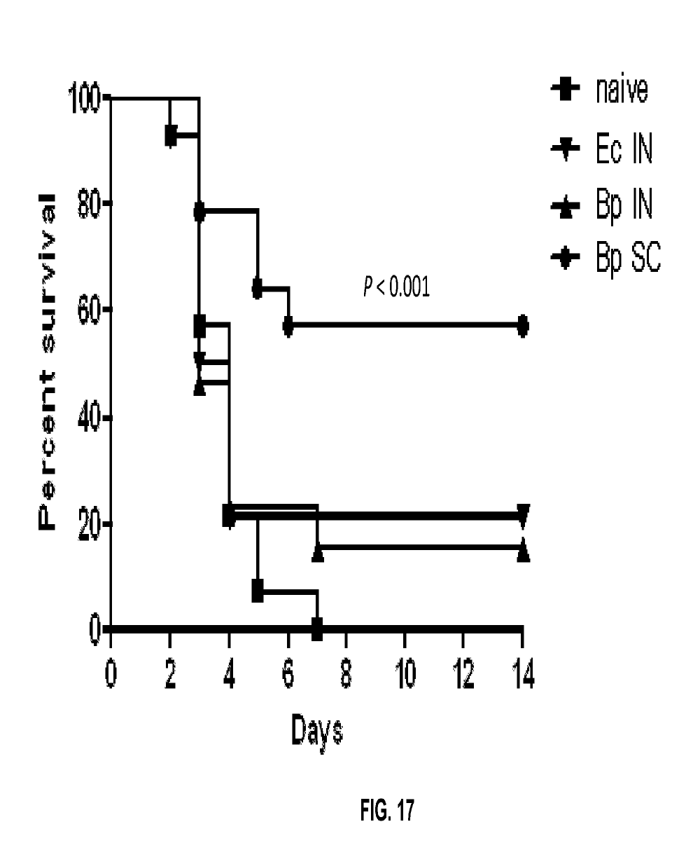

In order to determine if immunization with B. pseudomallei OMVs could provide protection against inhalational infection, groups of mice were immunized as above and challenged by aerosol with virulent B. pseudomallei strain 1026b. Two independent immunization and challenge experiments were performed with two separately prepared batches of OMV vaccine to demonstrate reproducibility. Naïve mice displayed 100% mortality by day 7 (FIG. 17). In contrast, mice immunized SC with B. pseudomallei OMVs were significantly protected against lethal aerosol challenge (P<0.001). No significant protection was observed in mice immunized IN with B. pseudomallei OMVs or E. coli OMVs although a small percentage of animals survived. The composite survival data for a 2 week period is shown since no animal succumbed after day 7. In addition, a portion of surviving animals was euthanized 2 weeks post-challenge for determination of bacterial burden.

(4) B. Pseudomallei OMV Immunization Reduces, but does not Completely Eliminate, Bacterial Persistence

Tissues known to harbor persistent B. pseudomallei (lung, liver, and spleen) were harvested from survivors after 14 and 30 days of observation and plated for determination of bacterial loads. Both groups of B. pseudomallei OMV-immunized mice (SC and IN) demonstrated absence of bacteria in the lungs by 14 days post-aerosol challenge (Table 4). In contrast, the E. coli OMV-immunized mice that survived challenge contained up to 106 CFU in their lungs on day 14. Two out of three B. pseudomallei OMV SC-immunized mice showed no evidence of B. pseudomallei in the spleen, and very low numbers of bacteria were detected in the liver (<30 CFU). As observed in the lung, E. coli OMV-immunized mice had higher numbers of B. pseudomallei in the spleen and liver compared to B. pseudomallei OMV-immunized animals at 14 days post-challenge. At 30 days post-challenge, a similar outcome was observed in that the E. coli OMV-immunized animal had higher CFU in all tissues compared to B. pseudomallei OMV immunized mice. We also noted low numbers of bacteria in the lungs of B. pseudomallei OMV immunized mice that contrasts with the lack of colonization seen at 14 days in these groups. These mice were also colonized with low numbers of bacteria in the spleen and/or liver. Bacterial recolonization of the lung from distant organs might have occurred after an extended period of infection, as B. pseudomallei possesses a tropism for the lung as described, for example, in Cheng A. C. et al., Clin Microbiol Rev, 18(2):383-416 (2005), the disclosure of which is incorporated herein by reference.

### (5) B. Pseudomallei OMV Immunization Induces High Titers of OMV-Specific Serum IgG and IgA

Antibody responses were measured in serum obtained from separate groups of mice one month after the last immunization in order to assess immune correlates of protection. B. pseudomallei OMV-specific serum IgG was significantly higher in the B. pseudomallei OMV SC- and IN-immunized animals than in controls (FIG. 18A). The concentrations of OMV-specific IgG were not significantly different between B. pseudomallei OMV SC- and IN-immunized mice. Furthermore, the concentrations of IgG1 and IgG2a were not significantly different between B. pseudomallei SC- and IN-immunized mice (Table 5). Both B. pseudomallei OMV SC- and IN-immunized groups demonstrated a Type 2 immune response with IgG1:IgG2a ratios equal to 7.5 and 12.2, respectively (Table 5). B. pseudomallei OMV-specific serum IgA was significantly higher in B. pseudomallei OMV IN-immunized mice compared to control groups (FIG. 18B). As noted in our initial immunogenicity studies, antibody responses to B. pseudomallei OMVs were specific since E. coli OMV-immunized mice did not produce antibodies that recognized B. pseudomallei OMVs, although they produced high titers of E. coli OMV-specific serum IgG and IgA (FIGS. 18C and 18D). Conversely, B. pseudomallei OMV-immunized mice did not generate a significant antibody response to E. coli OMVs (FIGS. 18C and 18D).

TABLE 4
B. pseudomallei OMV-immunized mice demonstrate reduced bacterial burdens.

| 14 days p.i. | | | | 30 days p.i. | | | |
|---|---|---|---|---|---|---|---|
| Group (n) | Lung | Liver | Spleen | Group (n) | Lung | Liver | Spleen |
| Ec IN (2) | 7 * 10²-2 * 10⁶ | 2 * 10²-6 * 10³ | 5 * 10²-1 * 10⁴ | Ec IN (1) | 3.5 * 10² | 2.6 * 10³ | 1.2 * 10² |
| Bp IN (1) | 0 | 1 * 10² | 3 * 10¹ | Bp IN (1) | 1 * 10¹ | 3 * 10¹ | 0 |
| Bp SC (3)^{a} | 0 | 1 * 10¹-3 * 10¹ | 3 * 10³ | Bp SC (3)^{b} | 1.3 * 10² | 6 * 10¹-2.9 * 10² | 1 * 10¹-5 * 10¹ |

Tissue bacterial burdens (CFU/organ) were determined in E. coli OMV-immunized (Ec IN), B. pseudomallei OMV Intranasally-immunized (Bp IN), and B. pseudomallei OMV Subcutaneously-immunized (Bp SC) mice at 14 and 30 days post-infection (p.i.). Three mice per group were utilized when possible. Number of mice (n) examined in each group is indicated in parentheses. Range in CFU recovered from replicate mice is reported above. a Only 1 mouse out of 3 was colonized in the spleen, therefore no range is provided. b Only 1 mouse out of 3 was colonized in the lung, therefore no range is provided.

TABLE 5
Mean serum B. pseudomallei OMV-specific IgG1 and
IgG2a concentrations (μg/ml) and IgG1:IgG2a ratios

| Group | IgG1 | IgG2a | Ratio |
|---|---|---|---|
| Naive | ND | 3.7 | — |
| Ec IN | ND | 7.1 | — |
| Bp IN | 413.0 | 33.9 | 12.2 |
| Bp SC | 324.5 | 43.2 | 7.5 |

Ratios >1 indicate a type 2 humoral immune response, while ratios <1 indicate a type 1 cellular immune response.
ND = non-detectable.

(6) Immunization with B. Pseudomallei OMVs Induces T Cell Memory Responses

A Th1-driven CMI response, in concert with the production of specific antibodies, is likely essential for vaccine efficacy against B. pseudomallei. See, for example, Haque, A. et al., J Infect Dis, 194(9):1241-8 (2006) and Healey, G. D. et al., Infect Immun, 73(9):5945-51 (2005), the disclosures of each of which are incorporated herein by reference. To assess antigen-specific T cell responses in OMV-immunized mice, spleens were harvested one month after the last immunization and re-stimulated ex vivo with B. pseudomallei OMVs. Cell culture supernatants were assayed on day three for IFN-γ_production as an indication of a Th1 memory response. Both groups of mice immunized with B. pseudomallei OMVs (SC and IN) produced significantly higher amounts of compared to control groups (FIG. 20). Similar to that observed for antibody responses, T cell memory responses to B. pseudomallei immunization appeared specific as splenocytes from E. coli OMV-immunized mice did not produce IFN-γ upon restimulation with B. pseudomallei OMVs.

### Discussion

The significant morbidity and mortality associated with Burkholderia pulmonary infection in humans calls for the development of a safe and efficacious vaccine against inhalational disease. Furthermore, a vaccine that provides sterile immunity would be especially useful since many members of the Burkholderia cause persistent infection. In this study, it was demonstrated that a naturally derived OMV vaccine provides inherent adjuvanticity, immunogenicity, and protective efficacy against a B. pseudomallei pulmonary challenge. Mice immunized SC with B. pseudomallei OMVs demonstrated nearly 60% survival against aerosol challenge compared to 0% survival in naïve animals. This study presents the best vaccine-mediated protection attained thus far against lethal pneumonic melioidosis in the mouse model. These results suggest membrane vesicles as a promising vaccine strategy against other respiratory pathogens, including those that establish persistent pulmonary infection such as Mycobacterium tuberculosis or the B. cepacia complex. Indeed, it was recently shown that M. tuberculosis produces vesicles that modulate immune responses and enhance bacterial virulence via TLR2 signaling. See, for example, Prados-Rosales R. et al., J Clin Invest, 121(4):1471-83 (2011), the disclosure of which is incorporated herein by reference.

Membrane vesicle-based vaccines offer numerous advantages to traditional vaccine strategies. For example, they are easy and inexpensive to produce—particularly native vesicles that do not require chemical treatment or other artificial modes of preparation. Membrane vesicles are non-viable yet share many of the surface antigens presented by an inactivated or live-attenuated strain without presenting the same safety concerns. Vesicles also contain numerous antigens that can influence immune responses, as described, for example, in Kulp, A. et al., Annu Rev Microbiol, 64:163-84 (2010) and Amano, A. et al., Microbes Infect, 12(11):791-8 (2010), the disclosures of each of which are incorporated herein by reference. This feature could overcome limitations associated with the use of a single antigen (i.e., LPS or protein subunit) and vaccine failure due to antigenic variance among heterogenous bacterial strains, escape mutants, and human leukocyte haplotype (HLA) restriction. See, for example, Sirisinha, S. et al., Microbiol Immunol, 42(11):731-7 (1998); Anuntagool, N. et al., Southeast Asian J Trop Med Public Health, 31(suppl. 1):146-52 (2000); Gal-Tanamy, M. et al., Proc Natl Acad Sci USA, 105(49):19450-5 (2008); Quenee, L. E. et al., Infect Immun., 76(5):2025-36 (2008); and Ovsyannikoa, I. G. et al., J Infect Dis, 193(5):655-63 (2006), the disclosures of each of which are incorporated herein by reference.

Using sensitive LC-MS analysis, numerous protein antigens in the purified vesicles were identified (Table 3).

TABLE 3
Protein composition of B. pseudomallei OMVs as determined by
LC-MS analysis. Proteins highlighted in bold are previously
identified immunogenic proteins [30, 35].

| | | | | |
|---|---|---|---|---|
| Putative | | | | Length |
| Cellular | | Accession | | (amino |
| Location | Gene | No. | Protein | acids) |

Cytoplasmic

| | | | | |
|---|---|---|---|---|
| 1 | BPSL0204 | 53717846 | ATP-dependent protease peptidase subunit | 178 |
| 2 | BPSL0212 | 53717854 | S-adenosylmethionine synthetase | 395 |
| 3 | BPSL0644 | 53718287 | aspartyl-tRNA synthetase | 598 |
| 4 | BPSL0793 | 53718432 | branched-chain amino acid | 307 |
| | | | aminotransferase | |
| 5 | BPSL0869 | 53718508 | ornithine carbamoyltransferase | 309 |
| 6 | BPSL1095 | 53718730 | transaldolase B | 317 |
| 7 | BPSL1207 | 53718843 | polynucleotide | 713 |
| | | | phosphorylase/polyadenylase | |
| 8 | BPSL1542 | 53719176 | cystathionine beta-lyase | 394 |
| 9 | BPSL1743 | 53719357 | arginine deiminase | 418 |
| 10 | BPSL1907 | 53719521 | dihydrolipoamide dehydrogenase | 476 |
| 11 | BPSL2148 | 53719757 | (3R)-hydroxymyristoyl-ACP dehydratase | 169 |
| 12 | BPSL2169 | 53719778 | 2,3,4,5-tetrahydropyridine-2,6-carboxylate | 275 |
| | | | N-succinyltransferase | |
| 13 | BPSL2342 | 53719952 | urocanate hydratase | 562 |
| 14 | BPSL2344 | 53719954 | histidine ammonia-lyase | 507 |
| 15 | BPSL2605 | 53720215 | thioredoxin reductase | 320 |
| 16 | BPSL2891 | 53720499 | putative glutathione S-transferase-like | 202 |
| | | | protein | |
| 17 | BPSL3216 | 53720824 | elongation factor G | 700 |
| 18 | BPSL3246 | 53720854 | N-acetyl-gamma-glutamyl-phosphate | 314 |
| | | | reductase | |
| 19 | BPSL3290 | 53720900 | S-adenosyl-L-homocysteine hydrolase | 473 |
| 20 | BPSS1715 | 53722736 | type II citrate synthase | 433 |
| 21 | BPSS2270 | 53723288 | dihydrolipoamide dehydrogenase | 466 |
| 22 | BPSL2697 | 53720307 | chaperonin GroEL, chromosome 1 | 546 |
| 23 | BPSS0477 | 53721514 | chaperonin GroEL, chromosome 2 | 546 |
| 24 | BPSL2258 | 53719868 | dihydrodipicolinate synthase | 300 |
| 25 | BPSS0580 | 53721616 | hypothetical protein | 411 |
| 26 | BPSL0893 | 53718533 | elongation factor G | 704 |
| 27 | BPSL0965 | 53718607 | leucyl aminopeptidase | 503 |
| 28 | BPSL1931 | 53719544 | hypothetical protein | 96 |
| 29 | BPSS1704 | 53722724 | aspartate-semialdehyde dehydrogenase | 373 |
| 30 | BPSL1413 | 53719049 | glucose-6-phosphate isomerase | 540 |
| 31 | BPSL1744 | 53719358 | ornithine carbamoyltransferase | 336 |
| 32 | BPSL1510 | 53719144 | nucleoside diphosphate kinase | 141 |
| 33 | BPSL1945 | 53719558 | threonyl-tRNA synthetase | 635 |
| 34 | BPSL0631 | 53718274 | YjgF family protein | 128 |
| 35 | BPSL1954 | 53719566 | succinyl-CoA:3-ketoacid-coenzyme A | 214 |
| | | | transferase subunit B | |
| 36 | BPSL2096 | 53719707 | putative hydroperoxide reductase | 183 |
| | | | (AhpC) | |
| 37 | BPSL2305 | 53719915 | oligopeptidase A | 700 |
| 38 | BPSL2390 | 53719996 | bifunctional N-succinyldiaminopimelate- | 411 |
| | | | aminotransferase/acetylornithine | |
| | | | transaminase protein | |
| 39 | BPSS0281 | 53721316 | 4-aminobutyrate aminotransferase | 428 |
| 40 | BPSS1356 | 53722381 | hypothetical protein | 1126 |
| 41 | BPSL0241 | 53717882 | ferredoxin--NADP reductase | 257 |
| 42 | BPSL1591 | 53719221 | hypothetical protein | 367 |
| 43 | BPSL2627 | 53720237 | 6,7-dimethyl-8-ribityllumazine synthase | 174 |
| 44 | BPSL1198 | 53718834 | ketol-acid reductoisomerase | 339 |
| 45 | BPSL2959 | 53720567 | malic enzyme | 770 |
| 46 | BPSL2160 | 53719769 | methionine aminopeptidase | 272 |
| 47 | BPSL3183 | 53720791 | delta-aminolevulinic acid dehydratase | 355 |
| 48 | BPSS0190 | 53721225 | O-acetylhomoserine | 432 |
| 49 | BPSL2953 | 53720561 | transketolase | 676 |
| 50 | BPSL3362 | 53720971 | glycine dehydrogenase | 976 |
| 51 | BPSL2507 | 53720116 | cysteine synthase B | 301 |
| 52 | BPSL3052 | 53720661 | anthranilate phosphoribosyltransferase | 344 |
| 53 | BPSS0044 | 53721083 | 3-oxoadipate CoA-transferase subunit B | 219 |
| 54 | BPSL1290 | 53718925 | thiamine biosynthesis protein | 644 |
| 55 | BPSS1888 | 53722909 | aromatic oxygenase | 419 |
| 56 | BPSS1448 | 53722471 | acyl-coA dehydrogenase | 394 |
| 57 | BPSS1726 | 53722747 | aconitate hydratase | 906 |
| 58 | BPSS0650 | 53721684 | peptidase | 484 |
| 59 | BPSL3124 | 53720731 | hypothetical protein | 249 |
| 60 | BPSL0372 | 53718012 | putative carbonic anhydrase | 257 |
| 61 | BPSL0547 | 53718186 | single-stranded DNA-binding protein | 185 |
| 62 | BPSL2194 | 53719803 | ribonuclease activity regulator protein | 166 |
| 63 | BPSL0118 | 53717759 | DNA topoisomerase III | 870 |
| 64 | BPSL0798 | 53718437 | fructose-1,6-bisphosphate aldolase | 355 |
| 65 | BPSL2192 | 53719801 | malate synthase | 531 |
| 66 | BPSL2612 | 53720222 | glucose-6-phosphate 1-dehydrogenase | 490 |
| 67 | BPSL2952 | 53720560 | glyceraldehyde 3-phosphate dehydrogenase 1 | 337 |
| 68 | BPSS2134 | 53723150 | hypothetical protein | 82 |
| 69 | BPSL2739 | 53720349 | homogentisate 1,2-dioxygenase | 451 |
| 70 | BPSS0081 | 53721120 | hypothetical protein | 140 |

Periplasmic

| | | | | |
|---|---|---|---|---|
| 1 | BPSL0249 | 53717890 | putative periplasmic dipeptide transport | 543 |
| | | | protein | |
| 2 | BPSL1555 | 53719189 | putrescine-binding periplasmic protein | 365 |
| | | | precursor | |
| 3 | BPSL2924 | 53720532 | glutamate/aspartate periplasmic binding | 301 |
| | | | protein precursor | |
| 4 | BPSS0802 | 53721827 | putative extracellular ligand binding protein | 381 |
| 5 | BPSS1103 | 53722131 | periplasmic thiamine binding protein | 477 |
| 6 | BPSS1249 | 53722272 | lipoprotein | 731 |
| 7 | BPSL3089 | 53720698 | peptidase | 722 |
| 8 | BPSL3416 | 53721023 | putative branched-chain amino acid ABC | 399 |
| | | | transporter periplasmic substrate binding | |
| | | | protein | |
| 9 | BPSS1722 | 53722743 | malate dehydrogenase | 328 |
| 10 | BPSL2544 | 53720154 | aminopeptidase N | 901 |
| 11 | BPSS0089 | 53721127 | hypothetical protein | 496 |
| 12 | BPSS0624 | 53721660 | macrolide-specific ABC-type efflux carrier | 654 |
| 13 | BPSS0136 | 53721172 | hypothetical protein | 463 |
| 14 | BPSL2701 | 53720311 | alcohol dehydrogenase | 336 |
| 15 | BPSS1992 | 53723013 | x-prolyl-dipeptidyl aminopeptidase | 645 |
| 16 | BPSL1082 | 53718718 | hypothetical protein | 156 |
| 17 | BPSS0046 | 53721085 | lactone hydrolase | 262 |

Outer Membrane/Extracellular

| | | | | |
|---|---|---|---|---|
| 1 | BPSS0580 | 53721616 | hypothetical protein | 412 |
| 2 | BPSS0767 | 53721793 | hypothetical protein | 108 |
| 3 | BPSL2403 | 53720009 | non-hemolytic phospholipase C precursor | 701 |
| 4 | BPSL2703 | 53720313 | hypothetical protein | 172 |
| 5 | BPSS0493 | 53721529 | chitin-binding protein | 366 |
| 6 | BPSS0564 | 53721600 | thermolysin metallopeptidase | 568 |
| 7 | BPSS0827 | 53721853 | peptidase | 646 |
| 8 | BPSL3297 | 53720907 | Gly/Ala/Ser-rich lipoprotein | 435 |
| 9 | BPSS1993 | 53723014 | serine metalloprotease precursor | 501 |
| 10 | BPSS1260 | 53722282 | hypothetical protein | 605 |
| 11 | BPSS0563 | 53721599 | aminopeptidase | 409 |
| 12 | BPSS1860 | 53722879 | hypothetical protein | 396 |

Several proteins appear to be highly abundant and immunogenic as determined by SDS-PAGE and Western blot, respectively (FIGS. 14A, 14C and 16B).

FIG. 14 demonstrates that OMVs shed by broth-grown B. pseudomallei contained immunoreactive antigens. (14A) SDS-PAGE and Coomassie stain of 5 mg purified OMVs. (14B) OMVs probed with pre-immune serum from a rhesus macaque or (14C) with convalescent serum obtained from the macaque 6 weeks post-infection with 1×10⁶ cfu B. pseudomallei 1026b (1:100 dilution; 2° antibody=goat anti-monkey IgG-HRP conjugated, 1:1000 dilution). MW=molecular weight protein ladder

FIG. 16 demonstrates that antibodies directed against multiple proteins are induced by OMV immunization. B. pseudomallei OMVs were probed with pooled sera obtained from (16A) naïve and (16B) OMV SC-immunized mice (n=5 per group) (1:100 dilution; 2° antibody=goat anti-mouse IgG-HRP conjugated, 1:1000 dilution). MW=molecular weight protein ladder Furthermore, multiple, independent batches of OMVs over a one-year period have been purified, and identical protein and immunogenicity profiles with each preparation was observed, which attests to the reproducibility of the product.

The safety and protective efficacy afforded by an OMV vaccine against N. meningitidis (Nm) serogroup B strains establishes precedence for use of such vaccines in the human population. See, for example, 12 (2009); Oster, P. et al., Vaccine, 23(17-18):2191-6 (2005); Oster, P. et al., Vaccine, 25(16):3075-9 (2007); and Boutriau D. et al., Clin Vaccine Immunol, (B:4:p 1.19, 15 and B:4:p 1.7-2, 4), 14(1):65-73 (2007), the disclosures of each of which are incorporated herein by reference. However, unlike B. pseudomallei OMVs, production of Nm-derived OMVs requires the removal of the extremely toxic lipooligosaccharide which necessitates the addition of aluminum hydroxide adjuvant to the OMV preparation to restore immunogenicity, as described, for example, in van de Waterbeemd, B. et al. (2010). Alum polarizes the immune response towards humoral and Th2 CMI, as described, for example, in Lindblad, E. B. et al., Immunol Cell Biol, 82(5):497-505 (2004), the disclosure of which is incorporated herein by reference, supporting the production of high titers of bactericidal antibody necessary for protection against meningococcus. Both humoral and Th1 CMI are likely essential for protection against B. pseudomallei. Because B. pseudomallei OMVs possess low toxicity yet retain adjuvanticity, B. pseudomallei OMVs in their native form was utilized without extraction of LPS or addition of an exogenous adjuvant. While not wishing to be bound by any particular theory, innate immune recognition of B. pseudomallei OMVs could mimic those to the intact organism since OMVs have been shown to contain LPS, lipoproteins, and CpG DNA and to activate TLRs. See, for example, Kulp, A. et al., Annu Rev Microbiol, 64:163-84 (2010); Amano, A. et al., Microbes Infect, 12(11):791-8 (2010); Deatherage, B. L. et al., Mol Microbiol, 72(6): 1395-407 (2009); and Bergman, M. A. et al., Infect Immun, 73(3):1350-6 (2005), the disclosures of each of which are incorporated herein by reference. Furthermore, the particulate nature of OMVs enables delivery of intrinsic TLR agonists and antigenic cargo to the same antigen presenting cell, which leads to more efficient antigen presentation. See, for example, Blander, J. M. et al., Nature, 440(7085):808-12 (2006), the disclosure of which is incorporated herein by reference. The homologous prime-boost immunization in the present study compared the traditional parenteral route of immunization to intranasal delivery. Because it has been proposed that B. pseudomallei may utilize the NALT as a portal of entry in murine melioidosis, it was expected that the IN route of immunization might better prevent mucosal infections through the priming and activation of local antimicrobial immunity. See, for example, Owen, S. J. et al., J Infect Dis, 199(12):1761-70 (2009), the disclosure of which is incorporated herein by reference.

This study surprising and unexpectedly found that significant protection was observed in mice immunized SC with B. pseudomallei OMVs, but not those immunized IN. These differences in protection could not be attributed to OMV-specific serum IgG responses because the concentrations were not significantly different between the two groups. This study demonstrated that purified OMVs from B. pseudomallei contain both LPS and CPS which may contribute to the protective efficacy of the OMV vaccine. The protective capacity of antibodies directed towards the O-antigen of LPS and CPS of B. pseudomallei has been demonstrated in multiple studies. See, for example, Jones S. M. et al., J Med Microbiol, 51(12):1055-62 (2002); Nelson M. et al., J Med Microbiol, 53(Pt 12):1177-82 (2004); Ngugi, S. A. et al, Vaccine, 28(47):7551-5 (2010); Zhang, S. et al., Clin Vaccine Immunol, 18(5):825-34 (2011), the disclosures of each of which are incorporated herein by reference. Notably, out of 47 monoclonal antibodies generated to protein, glycoprotein, and polysaccharide epitopes of B. pseudomallei, only those directed against LPS and CPS were strongly bactericidal and highly effective in protecting against intranasal B. pseudomallei infection. None of the monoclonal antibodies reacting to bacterial proteins showed prominent opsonic activity, suggesting that protein epitopes were less accessible on intact bacteria, as described, for example, in Zhang S. et al. (2011). Although OMV-specific serum IgG, IgG1, and IgG2a responses were similar for IN- and SC-immunized mice, the LPS- or CPS specific antibody induced by the OMV vaccine could vary between routes of immunization. In support of this, purified LPS from Brucella melitensis administered SC to mice induced higher levels of LPS-specific serum IgG and IgG3 compared to IN delivery and provided superior protection against Brucella infection in the lung. See, for example, Bhattacharjee, A. K. et al., Infect Immun, 74(10):5820-5 (2006), the disclosure of which is incorporated herein by reference. Thus, differences in antibody concentrations or subtypes specific for the LPS and/or CPS sub-components of the OMV could account for the observed differences in vaccine efficacy. These scenarios may help explain differences between resistant and susceptible groups of immunized mice and ultimately provide insight into mechanisms of immunity to B. pseudomallei.

CMI responses are also an essential component of vaccine protection against B. pseudomallei, particularly once the organism establishes intracellular residence, as described, for example, in Haque, A. et al., J Infect Dis, 193(3):370-9 (2006) and Healey, G. D. et al., Infect Immun, 73(9):5945-51 (2005), the disclosures of each of which are incorporated herein by reference. Histological analyses demonstrate B. pseudomallei within macrophages in the lung, liver, and spleen. See, for example, Wong K. T. et al., Pathology, 28(2):188-91 (1996) and Wong K. T. et al., Histopathology, 26(1):51-5 (1995), the disclosures of each of which are incorporated herein by reference. Vaccine-induced sterile immunity has been difficult to achieve, as described, for example, in Sarkar-Tyson M. et al., Clin Ther, 32(8):1437-45 (2010). Despite the small number of animals available for tissue burden assessment, both B. pseudomallei OMV SC- and IN-immunized mice demonstrated a reduction in B. pseudomallei tissue burden compared to control E. coli OMV immunized mice that survived challenge. While not wishing to be bound by any particular theory, this could reflect the significant production of IFN-γ observed in restimulated splenocytes in B. pseudomallei OMV-immunized animals. See, for example, Healey G. D. et Infect Immun, 73(9):5945-51 (2005) and Santanirand, P. et al., Infect Immun, 67(7):3593-600 (1999), the disclosures of each of which are incorporated herein by reference. Antigen-specific T cells, particularly CD4+ T cells, are important sources of IFN-γ, and are essential for host resistance to acute and chronic infection with B. pseudomallei. See, for example, Haque A. et al., J Infect Dis, 193(3):370-9 (2006), the disclosure of which is incorporated herein by reference. Notably, protection could not be attributed to IFN-γ production alone since the IN group succumbed to challenge. The frequency of T cells producing multiple cytokines (IFN-γ, TNF and IL-2), rather than IFN-γ alone, has been shown to correlate with protective vaccine responses against several intracellular pathogens including M. tuberculosis, Leishmania major, and Plasmodium falciparum. See, for example, Lindenstrom, T. et al., J Immunol, 182(12):8047-55 (2009); Darrah P. A. et al, Nat Med, 13(7):843-50 (2007); and Roestenberg M. et al., N Engl J Med, 361(5):468-77 (2009), the disclosures of each of which are incorporated herein by reference. OMVs can deliver virulence factors directly into the host cytoplasm via fusion of OMVs with lipid rafts in the host plasma membrane, as described, for example, in Bomberger J. M. et al., PLoS Pathog, 5(4):e1000382 (2009), the disclosure of which is incorporated herein by reference. Moreover, degradation of OMVs in lysosomal compartments has also been observed. See, for example, Amano A. et al., Microbes Infect, 12(11):791-8 (2010), the disclosure of which is incorporated herein by reference. These features may facilitate antigen presentation of OMV cargo via both MHC Class I and Class II, respectively. Thus, delineation of the role of single and multicytokine-producing CD8+ and CD4+ T-cells in response to the B. pseudomallei OMV vaccine could provide useful insight. See, for example, Lertmemongkolchai, G. et al., J Immunol, 166(2):1097-105 (2001), the disclosure of which is incorporated herein by reference. Inhalation of B. pseudomallei is a natural route of infection, and it represents the primary route of exposure in a deliberate biological attack. A B. pseudomallei vaccine would be efficacious against this route of infection. Immunization with OMVs provided significant protection in the BALB/c mouse model of acute pneumonic melioidosis. This study demonstrates that naturally derived OMVs is a safe, inexpensive, multi-antigen vaccine strategy against B. pseudomallei that promotes both humoral and CMI responses. The approach utilized in this work provides a foundation to further improve the B. pseudomallei OMV vaccine through, for example, optimization studies examining dose, delivery, and adjuvant formulations. Furthermore, the success achieved with non-optimized, native B. pseudomallei OMVs in this study provides an opportunity to extend vesicle-based vaccines to other clinically significant intracellular pathogens that have evaded traditional vaccination efforts.

### REFERENCES

1. Drevinek P, Mahenthiralingam E. Burkholderia cenocepacia in cystic fibrosis: epidemiology and molecular mechanisms of virulence. Clin Microbiol Infect 2010; 16(7):821-30.
2. Whitlock G C, Estes D M, Torres A G. Glanders: off to the races with Burkholderia mallei. FEMS Microbiol Lett 2007; 277(2):115-22.
3. Cheng A C, Currie B J. Melioidosis: epidemiology, pathophysiology, and management. Clin Microbiol Rev 2005; 18(2):383-416.
4. Wiersinga W J, van der Poll T, White N J, Day N P, Peacock S J. Melioidosis: insights into the pathogenicity of Burkholderia pseudomallei. Nat Rev Microbiol 2006; 4(4):272-82.
5. Dance D A. Melioidosis as an emerging global problem. Acta Trop 2000; 74(2-3):115-9.
6. Bondi S K, Goldberg J B. Strategies toward vaccines against Burkholderia mallei and Burkholderia pseudomallei. Expert Rev Vaccines 2008; 7(9):1357-65.
7. Sarkar-Tyson M, Titball R W. Progress toward development of vaccines against melioidosis: a review. Clin Ther 2010; 32(8):1437-45.
8. Atkins T, Prior R G, Mack K, Russell P, Nelson M, Oyston P C, et al. A mutant of Burkholderia pseudomallei, auxotrophic in the branched chain amino acid biosynthetic pathway, is attenuated and protective in a murine model of melioidosis. Infect Immun 2002; 70(9):5290-4.
9. Haque A, Chu K, Easton A, Stevens M P, Galyov E E, Atkins T, et al. A live experimental vaccine against Burkholderia pseudomallei elicits CD4+ T cell-mediated immunity, priming T cells specific for 2 type III secretion system proteins. J Infect Dis 2006; 194(9):1241-8.
10. Barnes J L, Ketheesan N. Development of protective immunity in a murine model of melioidosis is influenced by the source of Burkholderia pseudomallei antigens. Immunol Cell Biol 2007; 85(7):551-7.
11. Jones S M, Ellis J F, Russell P, Griffin K F, Oyston P C. Passive protection against Burkholderia pseudomallei infection in mice by monoclonal antibodies against capsular polysaccharide, lipopolysaccharide or proteins. J Med Microbiol 2002; 51(12):1055-62.
12. Nelson M, Prior J L, Lever M S, Jones H E, Atkins T P, Titball R W. Evaluation of lipopolysaccharide and capsular polysaccharide as subunit vaccines against experimental melioidosis. J Med Microbiol 2004; 53(Pt 12):1177-82.
13. Ngugi S A, Ventura V V, Qazi O, Harding S V, Kitto G B, Estes D M, et al. Lipopolysaccharide from Burkholderia thailandensis E264 provides protection in a murine model of melioidosis. Vaccine 2010; 28(47):7551-5.
14. Zhang S, Feng S H, Li B, Kim H Y, Rodriguez J, Tsai S, et al. In vitro and in vivo studies of monoclonal antibodies with prominent bactericidal activity against Burkholderia pseudomallei and Burkholderia mallei. Clin Vaccine Immunol 2011; 18(5):825-34.
15. Harland D N, Chu K, Haque A, Nelson M, Walker N J, Sarkar-Tyson M, et al. Identification of a LolC homologue in Burkholderia pseudomallei, a novel protective antigen for melioidosis. Infect Immun 2007; 75(8):4173-80.
16. Druar C, Yu F, Barnes J L, Okinaka R T, Chantratita N, Beg S, et al. Evaluating Burkholderia pseudomallei Bip proteins as vaccines and Bip antibodies as detection agents. FEMS Immunol Med Microbiol 2008; 52(1):78-87.
17. Hara Y, Mohamed R, Nathan S. Immunogenic Burkholderia pseudomallei outer membrane proteins as potential candidate vaccine targets. PLoS One 2009; 4(8):e6496.
18. Su Y C, Wan K L, Mohamed R, Nathan S. Immunization with the recombinant Burkholderia pseudomallei outer membrane protein Omp85 induces protective immunity in mice. Vaccine 2010; 28(31):5005-11.
19. Jeddeloh J A, Fritz D L, Waag D M, Hartings J M, Andrews G P. Biodefense-driven murine model of pneumonic melioidosis. Infect Immun 2003; 71(1):584-7.
20. Healey G D, Elvin S J, Morton M, Williamson E D. Humoral and cell-mediated adaptive immune responses are required for protection against Burkholderia pseudomallei challenge and bacterial clearance postinfection. Infect Immun 2005; 73(9):5945-51.
21. Kulp A, Kuehn M J. Biological functions and biogenesis of secreted bacterial outer membrane vesicles. Annu Rev Microbiol 2010; 64:163-84.
22. Amano A, Takeuchi H, Furuta N. Outer membrane vesicles function as offensive weapons in host-parasite interactions. Microbes Infect 2010; 12(11):791-8.
23. Deatherage B L, Lara J C, Bergsbaken T, Rassoulian Barrett S L, Lara S, Cookson B T. Biogenesis of bacterial membrane vesicles. Mol Microbiol 2009; 72(6): 1395-407.
24. Hoist J, Martin D, Arnold R, Huergo C C, Oster P, O'Hallahan J, et al. Properties and clinical performance of vaccines containing outer membrane vesicles from Neisseria meningitidis. Vaccine 2009; 27 (suppl. 2):B3-12.
25. Fransen F, Stenger R M, Poelen M C, van Dijken H H, Kuipers B, Boog C J, et al. Differential effect of TLR2 and TLR4 on the immune response after immunization with a vaccine against Neisseria meningitidis or Bordetella pertussis. PLoS One 2010; 5(12):e15692.
26. Alaniz R C, Deatherage B L, Lara J C, Cookson B T. Membrane vesicles are immunogenic facsimiles of Salmonella typhimurium that potently activate dendritic cells, prime B and T cell responses, and stimulate protective immunity in vivo. J Immunol 2007; 179(11):7692-701.
27. Schild S, Nelson E J, Camilli A. Immunization with Vibrio cholerae outer membrane vesicles induces protective immunity in mice. Infect Immun 2008; 76(10):4554-63.
28. Rivera J, Cordero R J, Nakouzi A S, Frases S, Nicola A, Casadevall A. Bacillus anthracis produces membrane-derived vesicles containing biologically active toxins. Proc Natl Acad Sci USA 2010; 107(44):19002-7.
29. Wedege E, Bolstad K, Aase A, Herstad T K, McCallum L, Rosenqvist E, et al. Functional and specific antibody responses in adult volunteers in New Zealand who were given one of two different meningococcal serogroup B outer membrane vesicle vaccines. Clin Vaccine Immunol 2007; 14(7):830-8.
30. Nieves W, Heang J, Asakrah S, Honer zu Bentrup K, Roy C J, Morici L A. Immunospecific responses to bacterial elongation factor Tu during Burkholderia infection and immunization. PLoS One 2010; 5(12):e14361.
31. Keller A, Nesvizhskii A I, Kolker E, Aebersold R. Empirical statistical model to estimate the accuracy of peptide identifications made by MS/MS and database search. Anal Chem 2002; 74(20):5383-92.
32. Keller A, Eng J, Zhang N, Li X J, Aebersold R. A uniform proteomics MS/MS analysis platform utilizing open XML file formats. Mol Syst Biol 2005; 1:0017, 2005.
33. Nuti D, Crump R, Handayani F, Chantratita N, Peacock S, Bowen R, et al. AuCoin. Identification of circulating bacterial antigens by in vivo microbial antigen discovery. mBio 2011; 2(4), e00136-11.
34. Morici L A, Heang J, Tate T, Didier P J, Roy C J. Differential susceptibility of inbred mouse strains to Burkholderia thailandensis aerosol infection. Microb Pathog 2010; 48(1):9-17.
35. Harding S V, Sarkar-Tyson M, Smither S J, Atkins T P, Oyston P C, Brown K A, et al. The identification of surface proteins of Burkholderia pseudomallei. Vaccine 2007; 25(14):2664-72.
36. Koeberling O, Seubert A, Granoff D M. Bactericidal antibody responses elicited by a meningococcal outer membrane vesicle vaccine with overexpressed factor H-binding protein and genetically attenuated endotoxin. J Infect Dis 2008; 198(2):262-70.
37. van de Waterbeemd B, Streefland M, van der Ley P, Zomer B, van Dijken H, Martens D, et al. Improved OMV vaccine against Neisseria meningitidis using genetically engineered strains and a detergent-free purification process. Vaccine 2010; 28(30):4810-6.
38. Utaisincharoen P, Tangthawornchaikul N, Kespichayawattana W, Anuntagool N, Chaisuriya P, Sirisinha S. Kinetic studies of the production of nitric oxide (NO) and tumour necrosis factor-alpha (TNF-alpha) in macrophages stimulated with Burkholderia pseudomallei endotoxin. Clin Exp Immunol 2000; 122(3): 324-9.
39. Matsuura M, Kawahara K, Ezaki T, Nakano M. Biological activities of lipopolysaccharide of Burkholderia (Pseudomonas) pseudomallei. FEMS Microbiol Lett 1996; 137(1):79-83.
40. Schaedler R W, Dubos R J. The susceptibility of mice to bacterial endotoxins. J Exp Med 1961; 113:559-70.
41. Prados-Rosales R, Baena A, Martinez L R, Luque-Garcia J, Kalscheuer R, Veeraraghavan U, et al. Mycobacteria release active membrane vesicles that modulate immune responses in a TLR2-dependent manner in mice. J Clin Invest 2011; 121(4):1471-83.
42. Sirisinha S, Anuntagool N, Intachote P, Wuthiekanun V, Puthucheary S D, Vadivelu J, et al. Antigenic differences between clinical and environmental isolates of Burkholderia pseudomallei. Microbiol Immunol 1998; 42(11):731-7.
43. Anuntagool N, Aramsri P, Panichakul T, Wuthiekanun V R, Kinoshita R, White N J, et al. Antigenic heterogeneity of lipopolysaccharide among Burkholderia pseudomallei clinical isolates. Southeast Asian J Trop Med Public Health 2000; 31(suppl. 1):146-52.
44. Gal-Tanamy M, Keck Z Y, Yi M, McKeating J A, Patel A H, Foung S K, et al. In vitro selection of a neutralization-resistant hepatitis C virus escape mutant. Proc Natl. Acad Sci USA 2008; 105(49):19450-5.
45. Quenee L E, Cornelius C A, Ciletti N A, Elli D, Schneewind O. Yersinia pestis caf1 variants and the limits of plague vaccine protection. Infect Immun 2008; 76(5):2025-36.
46. Ovsyannikova I G, Pankratz V S, Vierkant R A, Jacobson R M, Poland G A. Human leukocyte antigen haplotypes in the genetic control of immune response to measles-mumps-rubella vaccine. J Infect Dis 2006; 193(5):655-63.
47. Oster P, Lennon D, O'Hallahan J, Mulholland K, Reid S, Martin D. MeNZB: a safe and highly immunogenic tailor-made vaccine against the New Zealand Neisseria meningitidis serogroup B disease epidemic strain. Vaccine 2005; 23(17-18):2191-6.
48. Oster P, O'Hallahan J, Aaberge I, Tilman S, Ypma E, Martin D. Immunogenicity and safety of a strain-specific MenB OMV vaccine delivered to under 5-year olds in New Zealand. Vaccine 2007; 25(16):3075-9.
49. Boutriau D, Poolman J, Borrow R, Findlow J, Domingo J D, Puig-Barbera J, et al. Immunogenicity and safety of three doses of a bivalent (B:4:p 1.19, 15 and B:4:p 1.7-2, 4) meningococcal outer membrane vesicle vaccine in healthy adolescents. Clin Vaccine Immunol 2007; 14(1):65-73.
50. Lindblad E B. Aluminium compounds for use in vaccines. Immunol Cell Biol 2004; 82(5):497-505.
51. Bergman M A, Cummings L A, Barrett S L, Smith K D, Lara J C, Aderem A, et al. CD4+ T cells and toll-like receptors recognize Salmonella antigens expressed in bacterial surface organelles. Infect Immun 2005; 73 (3):1350-6.
52. Blander J M, Medzhitov R. Toll-dependent selection of microbial antigens for presentation by dendritic cells. Nature 2006; 440(7085):808-12.
53. Owen S J, Batzloff M, Chehrehasa F, Meedeniya A, Casart Y, Logue C A, et al. Nasal-associated lymphoid tissue and olfactory epithelium as portals of entry for Burkholderia pseudomallei in Murine Melioidosis. J Infect Dis 2009; 199(12):1761-70.
54. Bhattacharjee A K, Izadjoo M J, Zollinger W D, Nikolich M P, Hoover D L. Comparison of protective efficacy of subcutaneous versus intranasal immunization of mice with a Brucella melitensis lipopolysaccharide subunit vaccine. Infect Immun 2006; 74(10):5820-5.
55. Haque A, Easton A, Smith D, O'Garra A, Van Rooijen N, Lertmemongkolchai G, et al. Role of T cells in innate and adaptive immunity against murine Burkholderia pseudomallei infection. J Infect Dis 2006; 193(3):370-9.
56. Wong K T, Vadivelu J, Puthucheary S D, Tan K L. An immunohistochemical method for the diagnosis of melioidosis. Pathology 1996; 28(2): 188-91.
57. Wong K T, Puthucheary S D, Vadivelu J. The histopathology of human melioidosis. Histopathology 1995; 26(1):51-5.
58. Santanirand P, Harley V S, Dance D A, Drasar B S, Bancroft G J. Obligatory role of gamma interferon for host survival in a murine model of infection with Burkholderia pseudomallei. Infect Immun 1999; 67(7):3593-600.
59. Lindenstrom T, Agger E M, Korsholm K S, Darrah P A, Aagaard C, Seder R A, et al. Tuberculosis subunit vaccination provides long-term protective immunity characterized by multifunctional CD4 memory T cells. J Immunol 2009; 182(12):8047-55.
60. Darrah P A, Patel D T, De Luca P M, Lindsay R W, Davey D F, Flynn B J, et al. Multifunctional TH1 cells define a correlate of vaccine-mediated protection against Leishmania major. Nat Med 2007; 13(7):843-50.
61. Roestenberg M, McCall M, Hopman J, Wiersma J, Luty A J, van Gemert G J, et al. Protection against a malaria challenge by sporozoite inoculation. N Engl J Med 2009; 361 (5):468-77.
62. Bomberger J M, Maceachran D P, Coutermarsh B A, Ye S, O'Toole G A, Stanton B A. Long-distance delivery of bacterial virulence factors by Pseudomonas aeruginosa outer membrane vesicles. PLoS Pathog 2009; 5(4):e1000382.
63. Lertmemongkolchai G, Cai G, Hunter C A, Bancroft G J. Bystander activation of CD8+ T cells contributes to the rapid production of IFN-gamma in response to bacterial pathogens. J Immunol 2001; 166(2):1097-105.

### Example 9

The bacterium, B. pseudomallei (Bps), is the causative agent of melioidosis, a disease endemic in parts of Southeast Asia and Northern Australia. Bps is listed as a category B select agent due to its high lethality, innate resistance to antibiotics, and historical threat as a biological weapon. There is currently no effective vaccine against this organism. Gram-negative bacteria, including Bps, secrete outer membrane vesicles (OMVs), which are enriched with nucleic acids, lipids, and proteins. OMVs have been successfully utilized as a vaccine against serogroup B Neisseria meningitidis. The immunogenicity and protective efficacy of Bps-derived native OMVs (nOMVs) is described in the following exemplary study using BALB/c mice and an aerosol challenge model.

B. pseudomallei (Bps) is a Gram-negative, intracellular bacterium and the causative agent of melioidosis. The disease may manifest as acute septicemia, pneumonia and/or chronic infection and is associated with significant morbidity and mortality. Bps is naturally resistant to most antibiotics and there is currently no approved vaccine against systemic or inhalational infection. Previous vaccine strategies against Bps included inactivated whole cell preparations, live attenuated strains, subunit and DNA vaccines, amongst others, but none have achieved sterile immunity against high dose challenge. It has also been extremely difficult to protect against airborne infection. The immune responses believed to be protective against Bps infection include both antibody and cell-mediated immunity.

In this example, nOMVs prepared from Bps liquid culture were demonstrated as a novel vaccine candidate against pneumonic and septicemic melioidosis. In this exemplary work, the immunogenicity and protective efficacy of Bps-derived native OMVs (nOMVs) using BALB/c mice and an aerosol challenge model was tested.

### Methods:

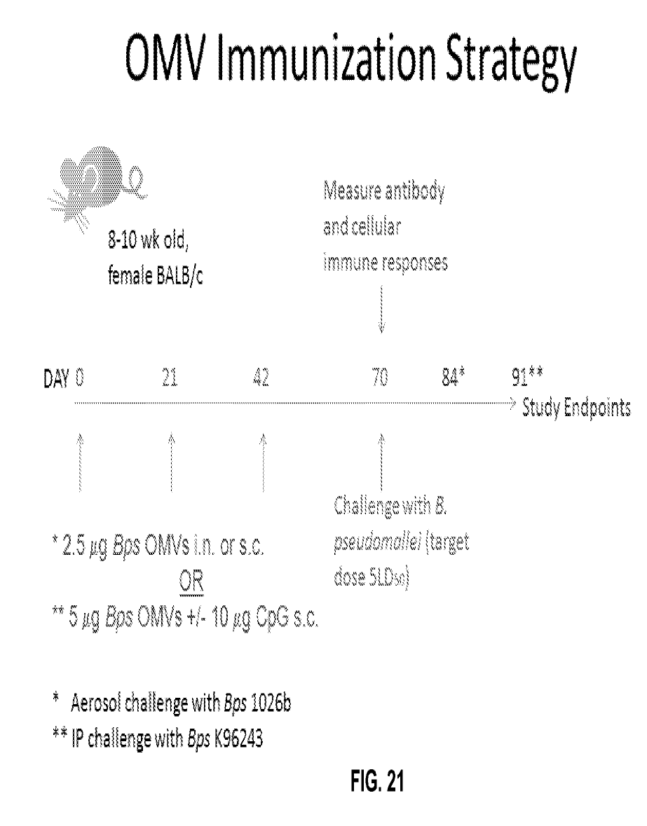

Bps and E. coli nOMVs were purified using density gradient centrifugation and visually confirmed by SDS-PAGE analysis and Cryo-Transmission Electron microscopy. Groups of BALB/c mice (n=20) were immunized subcutaneously (s.c.) or intranasally (i.n.) with 2.5 μg of Bps nOMV or E. coli nOMVs (i.n. only) without any exogenous adjuvant and boosted on days 21 and 42. Serial bleeds were performed over the course of immunization for measurement of serum antibody titers. Mice were challenged on day 70 with 5 LD₅₀ (1000 cfu) of Bps strain 1026b by aerosol. Survival was closely monitored for 2 weeks and tissues were harvested from survivors to determine bacterial burdens. An illustration of the exemplary OMV immunization strategy employed in this example is provided at FIG. 21.

In a separate experiment, mice were immunized s.c. with 5 μg of Bps nOMVs+/−10 μg CpG ODN and challenged intraperitoneally (i.p.) with 5 LD₅₀ (105 cfu) Bps strain K96243.

### Results:

Bps nOMVs were highly immunogenic in BALB/c mice and induced high titers of antigen-specific serum IgG after a single boost. No cross-reactive antibody was detected in serum from mice immunized with E. coli nOMVs. Significant protection against pneumonic melioidosis was achieved in mice vaccinated s.c. with Bps nOMVs. Protection against challenge with a heterologous strain of Bps was also achieved and was enhanced by the addition of CpG. LPS- and CPS-specific serum IgG and OMV-specific CD8⁺ memory T cells were significantly higher in protected groups of mice and represent immune correlates of protection to the OMV vaccine.

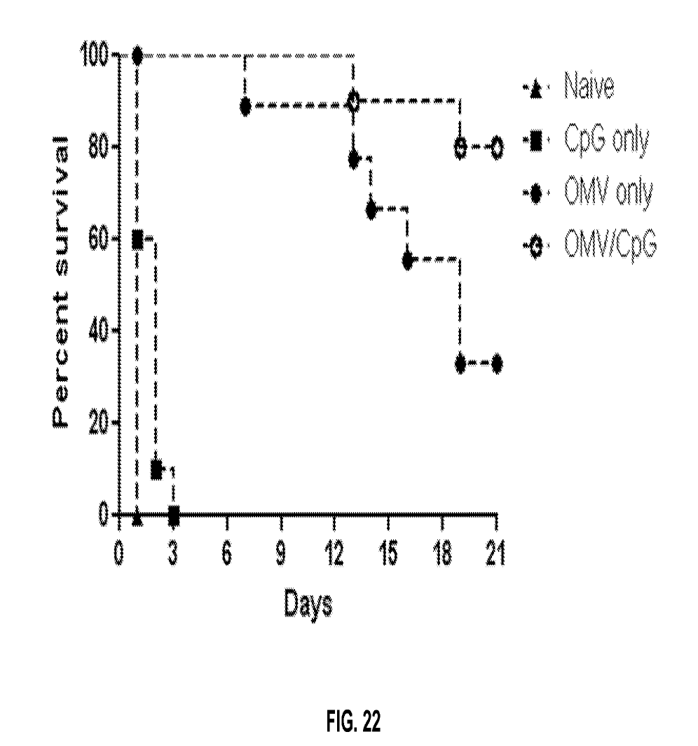

Mice immunized s.c. with Bps OMVs were significantly protected from pneumonic and septicemic melioidosis. At FIG. 22, the graph demonstrates that mice immunized with 2.5 mg OMVs s.c., but not i.n., were significantly protected from aerosol challenge. Mice that were immunized s.c. with 5 mg OMVs were significantly protected from i.p. challenge and protection was enhanced by the addition of CpG adjuvant. ** p<0.01; *** p<0.001.

Mice immunized with OMVs i.n. (Bp IN) and s.c. (Bp SC) produced similar levels of OMV-specific IgG and IgA. Microtiter plates were coated with Bps OMVs and serum antibody was measures by ELISA. Sera from naïve mice and those immunized with E. coli OMVs (Ec IN) did not react with Bps OMVs. *p<0.05

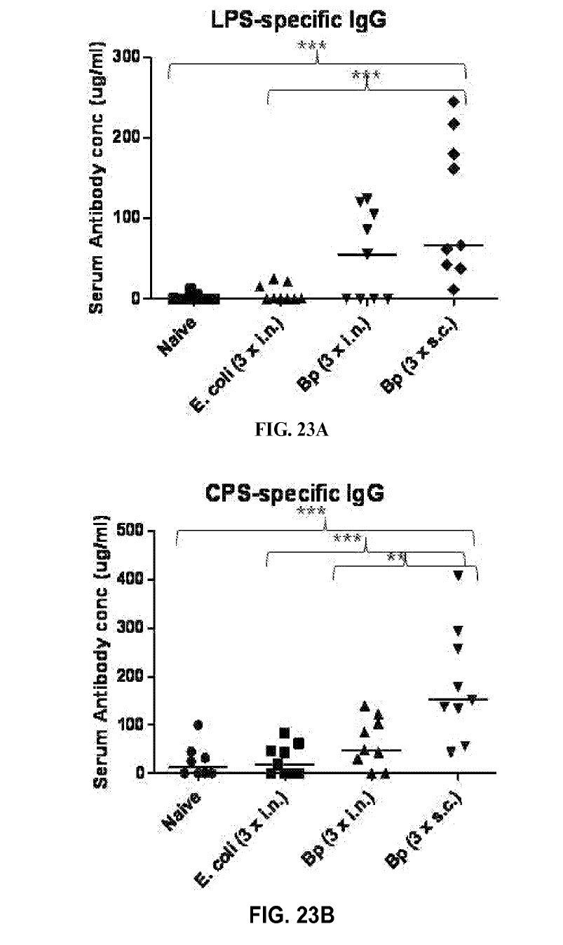

FIG. 23 demonstrates that mice immunized s.c. with Bps OMVs produced significantly higher concentrations of LPS-serum IgG (see FIG. 23A) and CPS-specific serum IgG (see FIG. 23B). Microtiter plates were coated with purified Bth LPS or Bps CPS and serum IgG was measured by ELISA. ** p<0.01; ***p<0.001

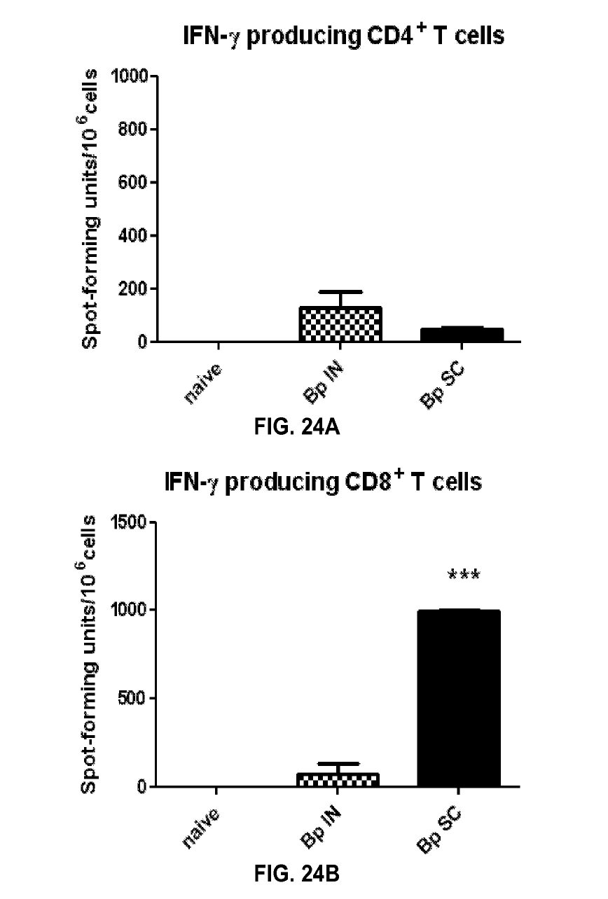

FIG. 24 demonstrates that mice IFN-γ-producing CD8+ T cells are significantly increased in mice immunized s.c. with Bps OMVs. Purified, splenic CD4⁺ (see FIG. 24A) and CD8⁺ T cells (see FIG. 24B) were re-stimulated with Bps OMVs and the frequency of IFN-γ producing cells was enumerated by ELlspot. *** p<0.001

B. pseudomallei nOMVs represent a safe, inexpensive, and efficacious vaccine against pneumonic and septicemic melioidosis. Protection in the Bps OMV s.c. immunized group is associated with high titers of LPS- and CPS-specific serum IgG and significantly higher IFN-γ-producing CD8⁺ T cells. The above study demonstrates that antibody and cellular immune responses to Bps nOMVs are specific. Further, this study demonstrates that addition of CpG ODN to the OMV vaccine enhanced protection.

### Example 10

### Prevention of Burkholderia Pulmonary Infection Using Bacterial-Derived Outer Membrane Vesicles

The opportunistic Burkholderia cepacia complex (Bcc), including B. cenocepacia and B. multivorans, has emerged as a significant cause of rapidly fatal pulmonary infection in individuals with cystic fibrosis (CF) in Europe, Canada, and the U.S. Preventive measures such as active immunization could dramatically reduce the incidence of disease yet there is currently no commercially available vaccine against any member of the Burkholderia. As demonstrated in the Examples above, immunization with Burkholderia pseudomallei-derived outer membrane vesicles (OMVs) provided significant protection against highly lethal pulmonary infection which was associated with rapid clearance of bacteria from the lungs. This Example describes OMVs as constituting a multi-antigen, safe, and inexpensive vaccine platform that can be rapidly developed to prevent Bcc lung infection in individuals with CF. It is imperative that innovative vaccine strategies, such as certain embodiments described herein, are utilized to halt the Bcc epidemic in the CF population.

Unlike most bacterial infections in patients with CF, infection with the Bcc can lead to a rapidly progressive necrotizing pneumonia known as “cepacia syndrome”. Due to its lethality, individuals colonized with Bcc may be restricted from attending CF social events or receiving a lung transplant. Considering the virulence of Bcc in CF individuals and its profound impact on their mental and physical well-being, prevention strategies targeting the Bcc are warranted. Indeed, the inherent multidrug resistance of the Bcc highlights the need for preventive rather than therapeutic options to reduce morbidity and mortality in CF.

B. pseudomallei (Bps), the causative agent of melioidosis, is a close-relative of the Burkholderia cepcia complex (Bcc), which includes B. cenocepacia and B. multivorans. The above Examples describe exemplary vaccine strategies against Bps utilizing outer membrane vesicles (OMVs). OMVs are constitutively shed from the surface of Gram-negative bacteria and contain numerous protective antigens, including polysaccharides and proteins. Immunization of mice with OMVs provided significant protection against pulmonary infection with Bps (see FIG. 17; (1)) and was associated with rapid clearance of bacteria from the lungs (1). The vaccine-mediated protection described in the Examples herein are surprisingly superior in comparison to other known non-living vaccine candidates against lethal pulmonary Bps infection in a mouse model. The Examples above provide basis for utilizing OMVs in vaccine strategies against other pathogenic Burkholderia. Described below are OMVs derived from B. multivorans (Bm) that will provide protection against pulmonary infection with Bm and will mediate cross-protection against other Bcc, such as B. cenocepacia (Bc).

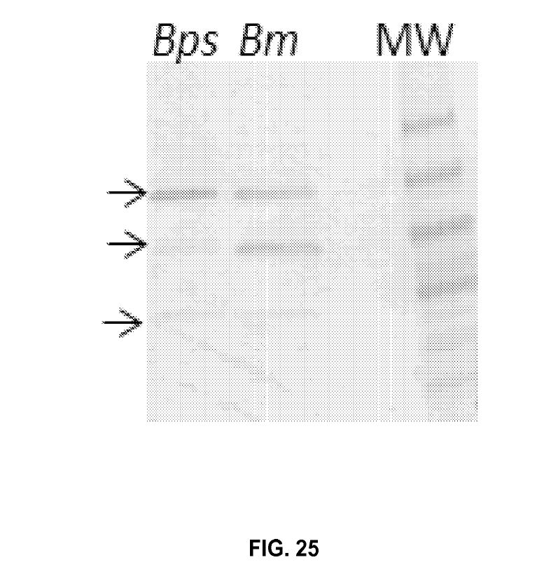

OMVs were purified from Bm and the presence of cross-reactive antigens in Bm and Bps using sera from mice immunized with Bps OMVs was confirmed by Western blot (FIG. 25, arrows). The presence of conserved antigens in the OMVs of these two Burkholderia species supports the likelihood of conserved antigens in other Bcc as well and the potential for OMV-mediated cross protection.

This Example describes the protective efficacy of Bm-derived OMVs against pulmonary Bm infection to be evaluated. OMVs purified from Bm will be used to immunize BALB/c mice. Mice will be challenged with Bm by the intranasal (i.n.) route and vaccine efficacy will be assessed by survival, bacterial burden, and histopathology. Mucosal and systemic OMV- and Bm-specific antibody responses will be measured.

This Example also describes the protective efficacy of Bm-derived OMVs against pulmonary Bc infection. Mice will be immunized with Bm OMVs as described above, but challenged with Bc to assess cross-protection.

### Methods:

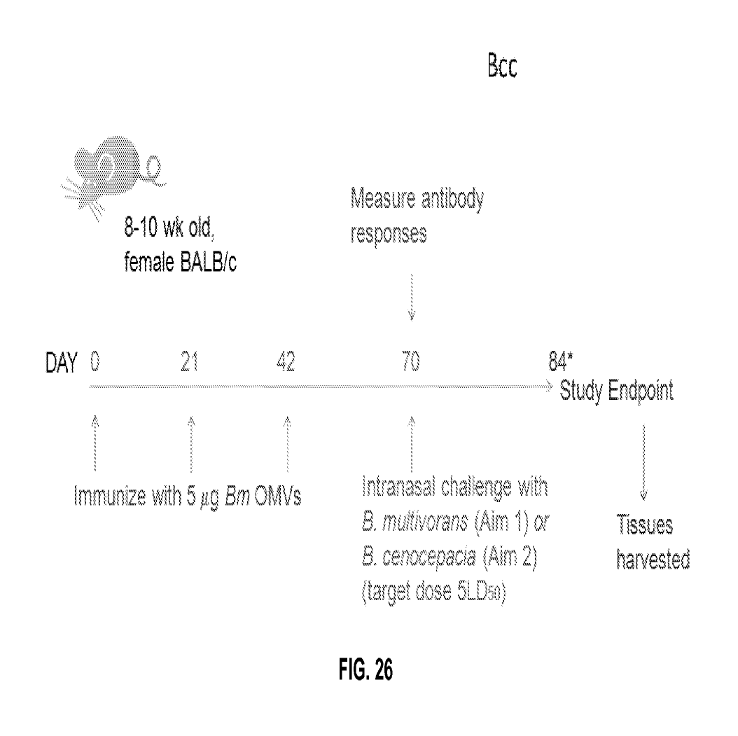

The experimental design is illustrated in FIG. 26. Mice (n=20) will be immunized subcutaneously with 5 μg of Bm-derived OMVs suspended in 100 μl PBS on day 0 and boosted on days 21 and 42. Sham-immunized mice (n=20) will receive PBS only. No exogenous adjuvant will be used since OMVs are highly immunogenic on their own. See, for example, Nieves et al., Vaccine, 29(46):8381-9 (2011), the disclosure of which is incorporated herein by reference. Four weeks after the last immunization, 10 mice from each group will be infected by the i.n. route with 5 LD₅₀ of Bm or Bc. The remaining 10 mice in each group will not be challenged but will be utilized for measurement of antigen-specific antibody responses. Infected mice will be monitored for survival for a two-week period.

Systemic and tissue bacterial burdens will be determined in euthanized animals and insurvivors sacrificed at the study endpoint by serial dilution plating of blood and tissue homogenates. Cytokine production will be measured in blood and lung homogenates using a Luminex multi-cytokine assay. Histopathology will be performed by a “blinded” Tulane pathologist who will score the sections on a graded scale as previously described, for example, in Morici, L. et al., Microbial pathogenesis, 48(1):9-17 (2010), the disclosure of which is incorporated herein by reference. Antigen-specific antibody will be measured in the sera and bronchoalveolar lavage fluid (BAL) of OMV-immunized and control animals on days 0 (pre-immune), 21, 42, and 70 to assess antibody responses over the course of immunization and prior to challenge. Antigen-specific IgG, IgG1, IgG2a, IgG3, and IgA will be measured by ELISA as previously described, for example, in Nieves et al. (2011). In order to determine the ability of antibodies generated to the OMV vaccine to promote bacterial killing, opsonophagocytic activity and complement-mediated killing of Bm and Bc will be assayed ex vivo as described, for example, in Ho et al., Infect Immun., 65(9):3648-53 (1997), the disclosure of which is incorporated herein by reference.

### Results:

Immunization with OMVs will provide significant protection against Bc and/or Bm which will be associated with rapid bacterial clearance, reduced histopathology and inflammation, and high titers of OMV-specific systemic and mucosal antibody. Larger scale efficacy study in CFTR knockout mice will be conducted and a non-human primate model of Burkholderia infection. The previous Examples presented herein describe purification of OMVs from Bm, Bps, and numerous other Gram-negative bacteria and further show these to be free of bacterial contamination so as to proceed with immunization studies. If immunization of mice with Bm-derived OMVs show reduced efficacy against heterologous challenge with Bc, then OMVs from Bc will be purified and used for immunization and challenge studies with Bc. A mixture of OMVs derived from various Bcc members could be utilized as a single vaccine formula to achieve broad-spectrum protection against the Burkholderia.

### REFERENCES

1. Nieves et al., Vaccine, 29(46):8381-9 (2011)
2. Morici, L. et al., Microbial pathogenesis, 48(1):9-17 (2010)
3. Ho et al., Infect Immun., 65(9):3648-53 (1997)

### Example 11

This Example describes use of OMV vaccines in a non-human primate (NHP) model of pneumonic melioidosis that was described in the exemplary Examples above. OMV immunization of rhesus macaques will induce protective antibody and CMI responses against Bps.

This Example describes protective immune responses to Bps OMV immunization in the rhesus macaque. Antigen-specific antibody, CD4+ and CD8+ T cells will be quantitated and compared in OMV- and sham-immunized animals (n=2 per group). Bactericidal antibody and effector T cell assays will also be performed ex vivo as a qualitative measure of immune responses.

This Example also describes protection of OMV-immunized macaques against aerosol challenge with Bps. Four weeks after immunization, the animals will be challenged with a lethal dose of Bps by aerosol. Survival and disease progression will be closely monitored for 21 days. Systemic and mucosal bacterial burdens, histopathology, and immune responses will be determined in euthanized animals and in survivors at the study endpoint.

OMV immunization of rhesus macaques will induce similar protective antibody and T cell responses to that previously observed in mice. Protective efficacy of the OMV vaccine in the model that most closely resembles human melioidosis will also be determined.

### Background

Bps is a major public health concern in the endemic regions of southeast Asia and northern Australia yet the organism has a worldwide distribution and cases are likely under-reported (1). In northeast Thailand, the mortality rate associated with Bps infection is over 40%, making it the 3rd most common cause of death from infectious disease in that region after HIV/AIDS and TB (2). The inherent resistance of Bps to multiple antibiotics impairs treatment, prompting aggressive prophylaxis for up to 6 months with relapse common (3-5). Beyond its public health significance, Bps is considered a potential biological warfare agent by the U.S. DHHS and was recently recommended for Tier 1 classification, a status also assigned to Yersinia pestis and Bacillus anthracis, among others. A protective vaccine against Bps is the best option to reduce morbidity and mortality in endemic areas and to provide a safeguard against biological attack with this organism because aggressive antibiotic treatment often fails, but no ideal candidate against Bps has yet emerged from preclinical studies.

A live-attenuated vaccine strain, 2D2, induces humoral and cellular mediated immune (CMI) responses and confers significant protection against systemic Bps challenge in mice (6), but Bps's ability to establish latent infection poses safety concerns for live vaccine applications. Vaccine formulations utilizing purified polysaccharides, recombinant proteins (i.e. Type 3 secretion system or outer membrane proteins) and DNA vaccines have shown only limited success, particularly against aerosol challenge (7-9). Furthermore, none of these vaccines achieved sterilizing immunity against high dose challenge with this persistent pathogen (10). By contrast, the above representative Examples herein demonstrated that immunization of mice with naturally derived OMVs provided significant protection against aerosol challenge with Bps. Data provided in the representative Examples above demonstrate that significant bacterial clearance in tissues known to harbor persistent Bps was achieved, which may be associated with the observed induction of CD4+ and CD8+ T cell responses to the OMVs. This observation is significant because non-living vaccine formulations often fail to elicit robust CD8+ T cell responses yet OMVs appear capable of doing so. The pursuit of vesicles as a vaccine platform is further supported by recent work showing vesicle-mediated delivery of virulence factors by M. tuberculosis (11) and B. anthracis (12), a process that is neither random nor passive but rather selective and directed by the bacteria (13). The representative Examples herein provide an innovative vaccine strategy against Bps that is safe, inexpensive, and efficacious against aerosol infection—a feat not yet achieved by any other vaccine candidate.

Vaccine platforms that are effective against intracellular bacterial pathogens remain a high priority. In addition to the global impact of intracellular bacterial infections on public health, the alarming increase in multidrug resistant strains, such as Mycobacterium tuberculosis, and the potential threat of biological attack with select agents, such as B. pseudomallei (Bps) and B. mallei, highlight the urgent need for safe and effective vaccines against this collective group of pathogens. A vaccine that can elicit a range of immune responses, including antibody, helper CD4+ and cytotoxic CD8+ T cells is especially desirable for bacteria that establish intracellular infection. The representative Examples above demonstrate that immunization with naturally-derived outer membrane vesicles (OMVs) could provide protection against an aerosolized, intracellular bacterium, Bps. Furthermore, OMV vaccines provided in the above representative Examples provide superior protection to any other Bps vaccine candidate tested thus far. OMVs are constitutively shed by gram-negative bacteria and are often enriched with numerous virulence factors and Toll-like receptor agonists, which make them ideal multi-antigen vaccine candidates. In support of this, data from the above representative Examples demonstrate that: (1) Bps OMVs contained the T-independent antigens, lipo- and capsular polysaccharide, as well as multiple immunogenic proteins that may have collectively contributed to protection; (2) OMV immunization induced antigen-specific humoral and cellular-mediated immune (CMI) responses in mice; and (3) OMV immunization protected highly susceptible BALB/c mice from lethal intraperitoneal and aerosol challenge with Bps.

The OMV vaccine work presented herein represents a departure from the status quo regarding the majority of OMV vaccine studies to date. Immunization studies using vesicles have addressed predominantly extracellular pathogens, such as N. meningitides (14), Vibrio cholerae (15), and B. anthracis (12) and have thus largely emphasized antibody-mediated protection. Other studies which utilized OMVs to express heterologous antigens or as vaccine delivery vehicles also targeted humoral immunity (16-18). In contrast, in one aspect of the invention, the representative Examples presented herein confirm that OMVs constitute a non-living, multi-antigen vaccine formulation that can induce antigen-specific antibody and T cell responses to an intracellular pathogen. OMVs can deliver virulence factors directly into the host cytoplasm via fusion of OMVs with lipid rafts in the host plasma membrane (19) but degradation of OMVs in lysosomal compartments has also been observed (20). These features may facilitate antigen presentation of OMV cargo via both MHC Class I and Class II, respectively. While others have shown that S. typhimurium OMVs elicit robust B and CD4+ T cell responses during infection (21, 22), the representative Examples presented herein demonstrates OMV induction of CD8+ T cells. MHC Class I and Class II presentation of OMV cargo is surprising, and highly advantageous benefit for use in a vaccine platform against intracellular bacteria. The use of OMVs to elicit cellular immunity is a novel and inventive contribution to the OMV field, and can be applied to other intracellular, persistent bacteria, such as M. tuberculosis, using their own homologous vesicles (11). Alternatively, studies using native vesicles can guide rational vaccine design of synthetic nanoparticles or liposomes engineered to express essential, protective antigens.

Despite enhanced research and vaccine efforts in recent years, traditional vaccine strategies employing attenuated bacterial strains, recombinant proteins, or purified lipo- or capsular-polysaccharide have failed to elicit complete protection against aerosol challenge with Bps (23). Inhalation represents the primary route of infection in a deliberate biological attack and it is imperative that vaccine candidates are efficacious against this route of challenge. The above representative Examples have demonstrated that highly susceptible BALB/c mice immunized with OMVs by the subcutaneous route (SC) displayed 60% survival against lethal Bps aerosol challenge (5 LD50, approx. 1000 cfu/lung) compared to 0% survival in naïve animals (see FIG. 17; (24)). This represents the best vaccine-mediated protection attained thus far by a non-living vaccine candidate against lethal pneumonic melioidosis in the mouse model.

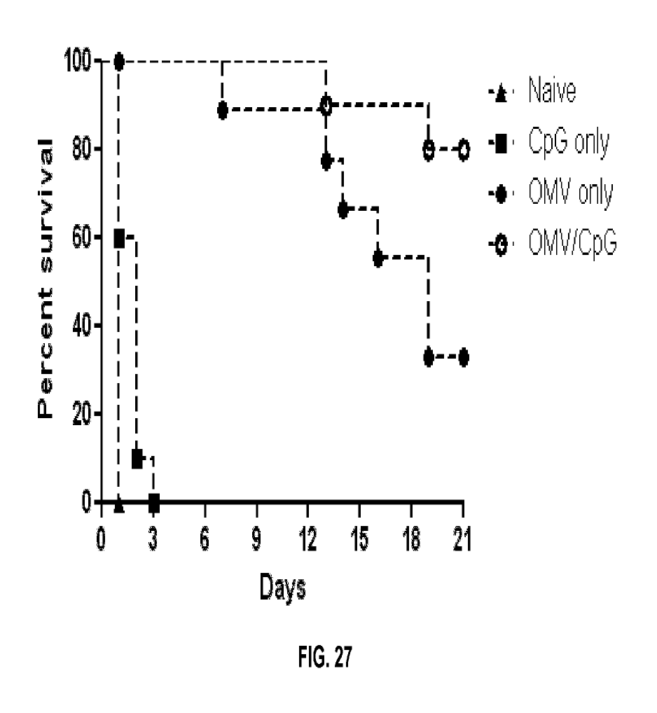

As described in the Examples above, embodiments of the OMV vaccine formulations presented herein contain no additional exogenous adjuvant and utilized a very low amount of antigen (2.5 μg OMV protein). The effects of adding an exogenous adjuvant, CpG ODN, and/or increasing the amount of antigen to enhance OMV protective capacity was evaluated. Mice immunized SC with 5 μg of OMVs were significantly protected against intraperitoneal (IP) challenge with 5 LD50 (approx. 8×105 cfu) of Bps K96243, while 20 out of 20 control mice succumbed within 72 hrs of challenge (see FIG. 27). Incorporation of CpG adjuvant into the OMV formula significantly improved protection (see FIG. 27). These data indicate that incorporation of CpG adjuvant improves OMV vaccine-mediated protection and provide evidence that OMVs derived from Bps strain 1026b provide protection against a heterologous Bps strain (K96243).

FIG. 27 demonstrates that CpG adjuvant improved OMV vaccine-mediated protection against Bps. Mice (n=10 per group) were challenged with 5 LD50 of Bps K96243 by IP injection. Mice immunized with 5 μg OMVs (derived from strain 1026b) or 5 μg OMVs admixed with 10 μg CpG ODS were significantly protected compared to control mice (mice that received CpG only or naïve mice) (***P<0.001; **P<0.01 using a log rank Mantel-Cox survival analysis). Note: Two mice in the OMV/CpG group were euthanized due to abscess formation at the site of injection and technically did not succumb to infection.

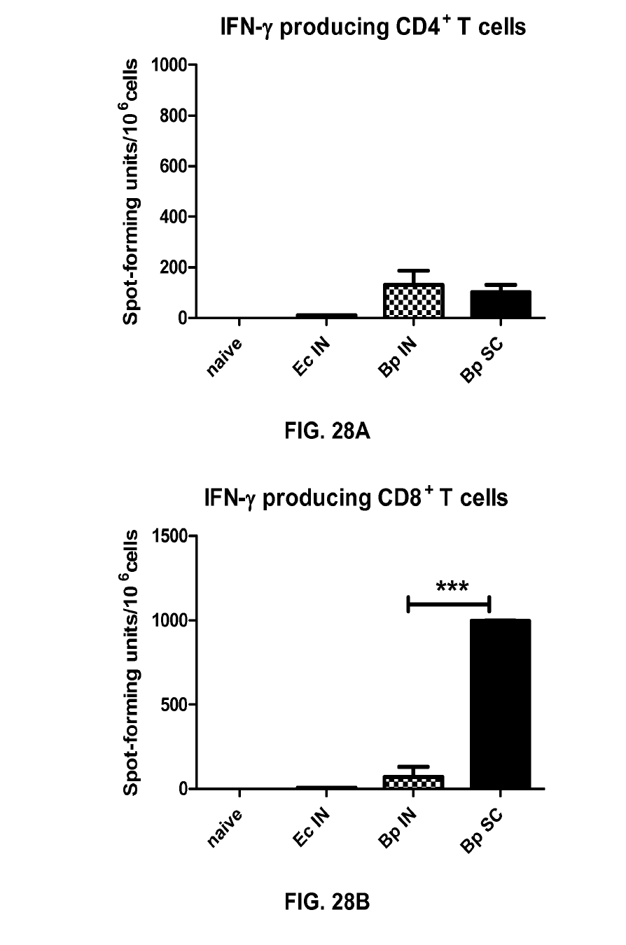

Unlike mice immunized SC, mice immunized intranasally (IN) with OMVs were not protected against Bps aerosol challenge (see FIG. 17; (24)). See, for example, Nieves, W. et al., Vaccine, 29:8381-8389 (2011), the disclosure of which is incorporated herein by reference. Differences in protection between SC- and IN-immunized mice could not be discriminated based upon total OMV-specific antibody responses. Nieves, W. et al. (2011) showed that Bps OMVs contain the protective antigens, lipopolysaccharide (LPS) and capsular polysaccharide (CPS), which suggests that these T-independent bacterial antigens may contribute to OMV-mediated vaccine protection. In support of this, mice immunized SC generated significantly higher concentrations of LPS- and CPS-specific serum IgG compared to controls as well as significantly higher concentrations of CPS-specific serum IgG compared to IN-immunized mice (see FIG. 28). These observations could account for the differences in protection observed in Bps OMV SC- and IN-immunized mice.

FIG. 28 demonstrates that OMV immunization induced protective LPS- and CPS-specific antibody. Mice were immunized IN (3×i.n.) or SC (3×s.c.) with 2.5 μg of Bps OMVs. Controls received nothing (naïve) or 2.5 μg OMVs. E. coli-derived OMVs (3×i.n.). Serum was obtained 1 month after the last immunization and used to measure LPS- or CPS-specific IgG by ELISA. ***p<0.001; **p<0.01 using a one-way ANOVA.

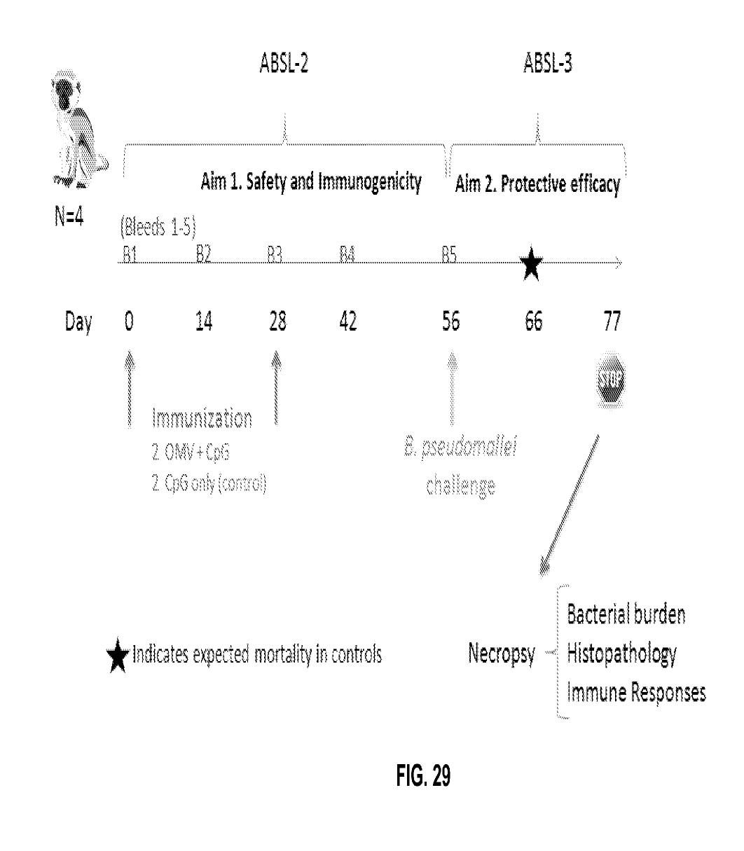

In addition, mice immunized SC exhibited significantly higher numbers of OMV-specific IFN-γ producing CD8+ T cells compared to non-protected groups (see FIG. 29). While not wishing to be bound by any particular theory, differences in survival observed between SC- and IN-immunized animals may indicate that LPS- and CPS-specific antibody and/or memory CD8+ T cells represent immune correlates of protection to the OMV vaccine. This will be further examined and characterized in the rhesus macaque.

FIG. 29 demonstrates that SC immunization with OMVs induced memory CD4+ and CD8+ T cells from immunized mice (n=5 per group) were re-stimulated with OMVs and the number of IFN-γ producing cells were enumberated by ELIspot. Unstimulated cells and PMA/ionomycin-stimulated cells were used as negative and positive controls respectively. ***p<0.001 using a one-way ANOVA

### Part 1—Evaluation of Protective Immune Responses to Bps OMV Immunization in the Rhesus Macaque

OMV vaccine-mediated protection has previously been shown to be largely antibody mediated which may be why extracellular bacterial pathogens have been predominantly targeted thus far. See, for example, references (15, 16, 25), the disclosures of each of which are incorporated herein by reference. This attribute of OMVs is advantageous against Bps because antibody responses in concert with CMI responses provide better protection against Bps than CMI alone. See, for example, reference (26), the disclosure of which is incorporated herein by reference. In addition to complement-activation, Fc receptor-mediated lysosomal targeting could enhance protection against Bps as demonstrated for other intracellular bacteria. See, for example, reference (27), the disclosure of which is incorporated herein by reference. Thus, antibody responses induced by OMV immunization may play a significant role in protection against Bps, especially during the early stages of disease. This is supported by both passive and active immunization studies which have shown that antibody specific for LPS or CPS can mediate protection to acute infection with Bps (7, 28-30).

As demonstrated in the representative Examples above, OMVs can also stimulate memory T cell responses in immunized mice, but the respective roles of CD4+ and CD8+ T cells in vaccine-mediated protection against Bps could be further elucidated to better understand the essential elements of acquired immunity. While not wishing to be bound by any particular theory, antibodies produced against LPS and CPS may confer short-term protective immunity while OMV proteins promote T cell dependent sterilizing immunity, which accounts for the effectiveness of the OMV vaccine. This Example evaluates the ability of OMVs to induce both humoral and CMI responses in rhesus macaques.

### Methods:

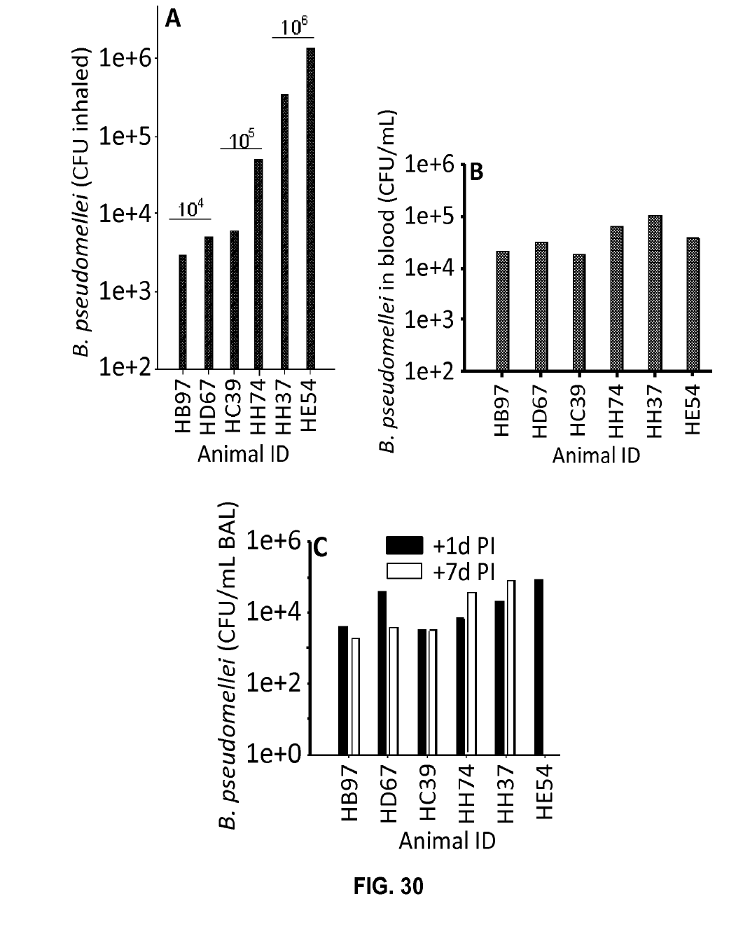

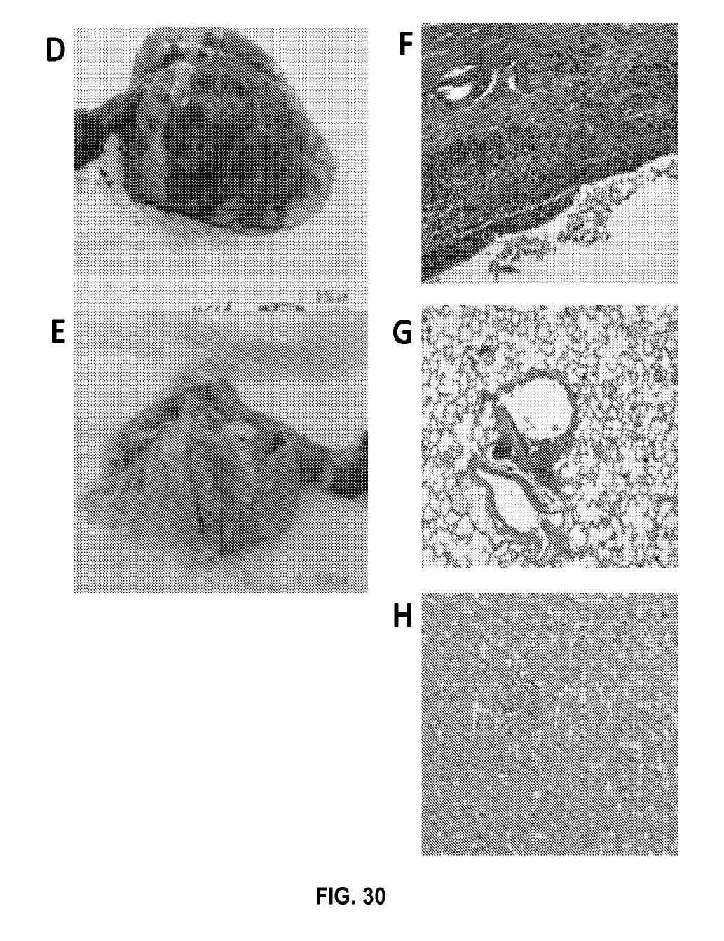

Four rhesus macaques will be utilized in this pilot study. Two animals will be immunized with 100 μg of OMV admixed with 400 μg CpG ODN 2395 and two animals will be given 400 μg CpG only. Others in the field have shown that CpG enhances vaccine protection against Bps. See, for example, references (8, 31, and 32), the disclosures of each of which are incorporated herein by reference. The amount of OMV vaccine administered to macaques is increased from the amount utilized in mice due to 1) the increase in body mass and 2) similar amounts of protein antigen/CpG have been shown to induce robust humoral and cellular immune responses in macaques. This approach is described, for example, in reference (33), the disclosure of which is incorporated herein by reference. The total amount of LPS administered as part of the OMV vaccine is 20 μg/dose, well below the endotoxin limits for NHP in pre-clinical research as described, for example, in reference (34), the disclosure of which is incorporated herein by reference. However, safety and toxicity of the OMV vaccine will be monitored by blood chemistry and by daily health observations. The experimental design for the study is illustrated in FIG. 30. Animals will be immunized on day 0 and boosted on day 28.

Antigen-specific antibody will be measured in the sera of OMV-immunized and control animals on days 0 (pre-immune), 14, 28, 42, and 56 to assess antibody responses over the course of immunization and prior to challenge. Antigen-specific serum IgM, IgG, and IgA will be measured separately by ELISA as previously described, for example, in references (24 and 35), the disclosures of each of which is incorporated herein by reference. Microliter plates will be coated with inactivated whole bacteria, purified OMV, LPS, or CPS, and antigen-specific antibody titers will be measured by serial dilution of sera. To determine the ability of antibodies generated to the OMV vaccine to promote bacterial killing, opsonophagocytic activity and complement-mediated killing of Bps will be assayed in vitro using sera obtained on days 0, 28, 42, and 56 as previously described, for example, in reference (36), the disclosure of which is incorporated herein by reference. Three individual experiments, each performed in triplicate, will be conducted.

Antigen-specific T cell responses to the OMV vaccine will be measured on days 0, 28, and 56 using PBMCs isolated from blood. PBMCs obtained from immunized and control animals will be re-stimulated with inactivated whole bacteria or purified OMVs, and the number and frequency of single- and multi-cytokine (IFN-γ, TNF-α, IL-2)-producing CD4+ and CD8+ T cells will be determined by intracellular cytokine staining and flow cytometry with the assistance of the TNPRC Immunology Core. In a parallel experiment, PBMCs will be sorted using a FACS-Aria cell sorter to isolate CD4+ and CD8+ T cells. Isolated T cells will be co-cultured with primate macrophages (derived from day 0 PBMCs) that have been infected with Bps and killing of intracellular bacteria will be measured to assess T cell effector responses as previously described, for example, in (37). Three individual experiments, each performed in triplicate, will be conducted.

Immunogenicity and safety of OMVs in NHPs will be demonstrated to corroborate that both humoral and cellular immune responses are elicited by the OMV vaccine. Specifically, high titers (>1:1000) of OMV-, LPS- and CPS-specific IgG in the serum of OMV-immunized animals by day 42 will be detected. This will indicate a robust humoral immune response in these animals which may provide protection against subsequent aerosol challenge with Bps. OMV immunization will also stimulate antigen-specific cellular immune responses by day 56. Specifically, an increase in the number of IFN-γ or triple-cytokine-producing CD4+ and CD8+ T cells in response to OMV immunization will be observed. Recent evidence has suggested that helper T cells capable of producing multiple antimicrobial and proliferative cytokines TNF-α, IL-2) in the same cell are the best correlate of protection for effective vaccination against a variety of intracellular pathogens including Leishmania major, M. tuberculosis, and Plasmodium falciparum. See, for example, references (38-40), the disclosures of each of which are incorporated herein by reference. OMV induction of effector memory T cells will eliminate intracellular bacteria as assessed in the ex vivo co-culture assay and by enumeration of tissue bacterial burdens.

Four bleeds prior to challenge will be implemented to assess antibody responses to the OMV vaccine. If a significant IgG response is not seen by day 42, a second boost will be administered on day 56. Additionally, the amount of antigen will be increased if no toxicity has been observed with the first two doses of vaccine and/or incorporate aluminum hydroxide as an adjuvant in order to boost antibody titers, so as to increase the likelihood of protection.

Part 2—Evaluation of Protection of OMV-Immunized Macaques Against Aerosol Challenge with Bps

The OMV multi-antigen vaccine preparation described in the representative Examples herein unexpectedly and surprisingly provide superior protection against Bps aerosol challenge in comparison to other known vaccines tested in the murine model. See, for example, reference (24), the disclosure of which is incorporated herein by reference. OMV vaccination will confer protection against acute pneumonic melioidosis in rhesus macaques. Further, OMV immunization will reduce or eliminate bacterial persistence and pathology in the lungs, livers, and spleens of infected animals.

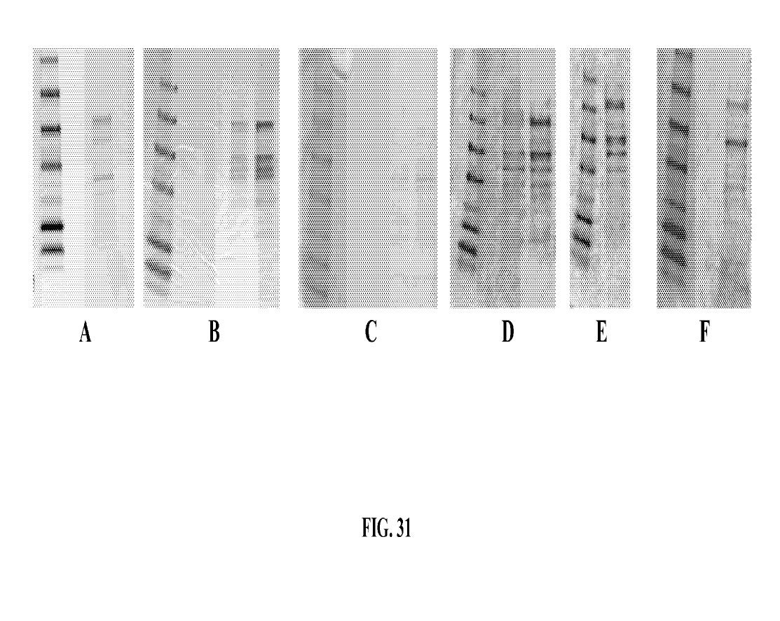

A study with six rhesus macaques was performed to establish the lethal dose for aerosolized Bps strain 1026b in these animals. Rhesus macaques were challenged with 104-106 cfu of aerosolized Bps (see FIG. 31A). Macaques that received 105 cfu displayed signs and symptoms of infection yet ultimately survived challenge. In contrast, macaques that received 106 cfu had rapid onset of illness and succumbed within 7-10 days of challenge. All animals possessed similar numbers of bacteria in the blood and bronchoalveolar lavage (BAL) fluid within 7 days post-exposure (FIGS. 31 B and 31C), but only animals challenged with 106 cfu developed pulmonary hemorrhage and systemic pathology (FIG. 31D-H). The disease progression and gross pathology observed in experimentally-infected rhesus macaques is consistent with that reported for a naturally-infected macaque and closely resembles human melioidosis, thus providing a highly relevant animal model to evaluate vaccine efficacy. See, for example, references (4) and (41), the disclosures of each of which are incorporated herein by reference. FIG. 31 demonstrates the effects of primates exposed by aerosol to B. pseudomallei 1026b at three target doses: (A) with significant bacteria in the blood; by +1d PI (B); and in BAL (C) at +1d and +7d PI. Lungs show signs of hemorrhage from an animal succumbing to disease at +7d PI (D). Animal exposed to approximately <1 log in challenge dose shows less trauma to lung (E). Histopathological analysis indicates focal tracheal necrosis Histopathological analysis indicates focal tracheal necrosis (F), lymphoid hyperplasia (G), and focal inflammation in the liver (H).

### Methods:

Four weeks after the last immunization, the animals from Part 1 of this Example will be challenged with Bps by aerosol. Macaques will be challenged with 2×106 cfu in order to achieve lethality in control animals within 10 days (day 66 in FIG. 31 above). Disease progression and survival will be monitored for 21 days. Systemic and tissue bacterial burdens will be determined in euthanized animals and in survivors sacrificed at the study endpoint by serial dilution plating of blood, BAL fluid, and tissue homogenates. Complete (RBL) necropsy will be performed by a TNPRC veterinary pathologist. Cytokine production will be measured in blood, BAL and lung homogenates of euthanized animals and in survivors at the study endpoint using a Luminex multi-cytokine assay. The primary endpoint for establishment of vaccine protective efficacy is survival of immunized animals compared to controls. Secondary endpoints include increased median time to death and/or reduction in tissue pathology and bacterial burden. Both qualitative and quantitative measurements of Bps-specific antibody and T cells will be performed in Part 1 of this Example to assess the potential for protection and to adjust immunization regimes accordingly.

OMV immunization will provide some level of protection in macaques, which may manifest as survival, delayed time to death, reduced pathology, and/or reduction in bacterial burdens. Furthermore, the outcome for each animal will be evaluated in the context of their individual immune responses which will help elucidate immune correlates of resistance versus susceptibility.

### REFERENCES

1. Currie B J, Fisher D A, Howard D M, Burrow J N, Selvanayagam S, Snelling P L, et al. The epidemiology of melioidosis in Australia and Papua New Guinea. Acta Trop. 2000; 74(2-3):121-7. PubMed PMID: 10674639.
2. Limmathurotsakul D, Wongratanacheewin S, Teerawattanasook N, Wongsuvan G, Chaisuksant S, Chetchotisakd P, et al. Increasing incidence of human melioidosis in Northeast Thailand. Am J Trop Med Hyg. 2010; 82(6):1113-7. Epub 2010 Jun. 4. doi: 10.4269/ajtmh.2010.10-0038. PubMed PMID: 20519609; PubMed Central PMCID: PMC2877420.
3. Limmathurotsakul D, Wuthiekanun V, Wongsuvan G, Pangmee S, Amornchai P, Teparrakkul P, et al. Repeat blood culture positive for B. pseudomallei indicates an increased risk of death from melioidosis. Am J Trop Med Hyg. 2011; 84(6):858-61. Epub 2011 Jun. 3. doi: 10.4269/ajtmh.2011.10-0618. PubMed PMID: 21633019; PubMed Central PMCID: PMC3110378.
4. Limmathurotsakul D, Peacock S J. Melioidosis: a clinical overview. Br Med Bull. 2011; 99:125-39. Epub 2011 May 12. doi: 10.1093/bmb/ldr007. PubMed PMID: 21558159.
5. Chaowagul W, Chierakul W, Simpson A J, Short J M, Stepniewska K, Maharjan B, et al. Open-label randomized trial of oral trimethoprim-sulfamethoxazole, doxycycline, and chloramphenicol compared with trimethoprim-sulfamethoxazole and doxycycline for maintenance therapy of melioidosis. Antimicrob Agents Chemother. 2005; 49(10):4020-5. PubMed PMID: 16189075.
6. Atkins T, Prior R G, Mack K, Russell P, Nelson M, Oyston P C, et al. A mutant of Burkholderia pseudomallei, auxotrophic in the branched chain amino acid biosynthetic pathway, is attenuated and protective in a murine model of melioidosis. Infect Immun. 2002; 70(9):5290-4. PubMed PMID: 12183585.
7. Nelson M, Prior J L, Lever M S, Jones H E, Atkins T P, Titball R W. Evaluation of lipopolysaccharide and capsular polysaccharide as subunit vaccines against experimental melioidosis. J Med Microbiol. 2004; 53(Pt 12):1177-82. PubMed PMID: 15585494.
8. Harland D N, Chu K, Haque A, Nelson M, Walker N J, Sarkar-Tyson M, et al. Identification of a LolC homologue in Burkholderia pseudomallei, a novel protective antigen for melioidosis. Infect Immun. 2007; 75(8):4173-80. PubMed PMID: 17517877.
9. Chen Y S, Hsiao Y S, Lin H H, Yen C M, Chen S C, Chen Y L. Immunogenicity and anti-Burkholderia pseudomallei activity in Balb/c mice immunized with plasmid DNA encoding flagellin. Vaccine. 2006; 24(6):750-8. PubMed PMID: 16169637.
10. Sarkar-Tyson M, Titball R W. Progress toward development of vaccines against melioidosis: A review. Clin Ther. 2010; 32(8):1437-45. Epub 2010 Aug. 24. doi: 10.1016/j.clinthera.2010.07.020. PubMed PMID: 20728758.
11. Prados-Rosales R, Baena A, Martinez L R, Luque-Garcia J, Kalscheuer R, Veeraraghavan U, et al. Mycobacteria release active membrane vesicles that modulate immune responses in a TLR2-dependent manner in mice. J Clin Invest. 2011; 121(4):1471-83. Epub 2011 Mar. 3. doi: 10.1172/JCI44261. PubMed PMID: 21364279; PubMed Central PMCID: PMC3069770.
12. Rivera J, Cordero R J, Nakouzi A S, Frases S, Nicola A, Casadevall A. Bacillus anthracis produces membrane-derived vesicles containing biologically active toxins. Proc Natl Acad Sci USA. 2010; 107(44):19002-7. Epub 2010 Oct. 20. doi: 10.1073/pnas.1008843107. PubMed PMID: 20956325; PubMed Central PMCID: PMC2973860.
13. Haurat M F, Aduse-Opoku J, Rangarajan M, Dorobantu L, Gray M R, Curtis M A, et al. Selective sorting of cargo proteins into bacterial membrane vesicles. J Biol Chem. 2011; 286(2):1269-76. Epub 2010 Oct. 9. doi: 10.1074/jbc.M110.185744. PubMed PMID: 21056982; PubMed Central PMCID: PMC3020734.
14. Holst J, Martin D, Arnold R, Huergo C C, Oster P, O'Hallahan J, et al. Properties and clinical performance of vaccines containing outer membrane vesicles from Neisseria meningitidis. Vaccine. 2009; 27 Suppl 2:B3-12. PubMed PMID: 19481313.
15. Schild S, Nelson E J, Camilli A. Immunization with Vibrio cholerae outer membrane vesicles induces protective immunity in mice. Infect Immun. 2008; 76(10):4554-63. PubMed PMID: 18678672.
16. Muralinath M, Kuehn M J, Roland K L, Curtiss R, 3rd. Immunization with Salmonella enterica serovar Typhimurium-derived outer membrane vesicles delivering the pneumococcal protein PspA confers protection against challenge with Streptococcus pneumoniae. Infect Immun. 2011; 79(2):887-94. Epub 2010 Dec. 1. doi: 10.1128/IAL00950-10. PubMed PMID: 21115718; PubMed Central PMCID: PMC3028854.
17. Chen D J, Osterrieder N, Metzger S M, Buckles E, Doody A M, DeLisa M P, et al. Delivery of foreign antigens by engineered outer membrane vesicle vaccines. Proc Natl Acad Sci USA. 2010; 107(7):3099-104. Epub 2010 Feb. 6. doi: 10.1073/pnas.0805532107. PubMed PMID: 20133740; PubMed Central PMCID: PMC2840271.
18. Kim J Y, Doody A M, Chen D J, Cremona G H, Shuler M L, Putnam D, et al. Engineered bacterial outer membrane vesicles with enhanced functionality. J Mol Biol. 2008; 380(1):51-66. PubMed PMID: 18511069.
19. Bomberger J M, Maceachran D P, Coutermarsh B A, Ye S, O'Toole G A, Stanton B A. Long-distance delivery of bacterial virulence factors by Pseudomonas aeruginosa outer membrane vesicles. PLoS Pathog. 2009; 5(4):e1000382. PubMed PMID: 19360133.
20. Amano A, Takeuchi H, Furuta N. Outer membrane vesicles function as offensive weapons in host-parasite interactions. Microbes Infect. 2010; 12(11):791-8. Epub 2010 Aug. 6. doi: 10.1016/j.micinf.2010.05.008. PubMed PMID: 20685339.
21. Alaniz R C, Deatherage B L, Lara J C, Cookson B T. Membrane vesicles are immunogenic facsimiles of Salmonella typhimurium that potently activate dendritic cells, prime B and T cell responses, and stimulate protective immunity in vivo. J Immunol. 2007; 179(11):7692-701. PubMed PMID: 18025215.
22. Bergman M A, Cummings L A, Barrett S L, Smith K D, Lara J C, Aderem A, et al. CD4+ T cells and toll-like receptors recognize Salmonella antigens expressed in bacterial surface organelles. Infect Immun. 2005; 73(3):1350-6. PubMed PMID: 15731032.
23. Bondi S K, Goldberg J B. Strategies toward vaccines against Burkholderia mallei and Burkholderia pseudomallei. Expert Rev Vaccines. 2008; 7(9):1357-65. PubMed PMID: 18980539.
24. Nieves W, Asakrah S, Qazi O, Brown K A, Kurtz J, Aucoin D P, et al. A naturally derived outer-membrane vesicle vaccine protects against lethal pulmonary Burkholderia pseudomallei infection. Vaccine. 2011. Epub 2011 Aug. 30. doi: 10.1016/j.vaccine.2011.08.058. PubMed PMID: 21871517.
25. Oster P, Lennon D, O'Hallahan J, Mulholland K, Reid S, Martin D. MeNZB: a safe and highly immunogenic tailor-made vaccine against the New Zealand Neisseria meningitidis serogroup B disease epidemic strain. Vaccine. 2005; 23(17-18):2191-6. PubMed PMID: 15755593.
26. Healey G D, Elvin S J, Morton M, Williamson E D. Humoral and cell-mediated adaptive immune responses are required for protection against Burkholderia pseudomallei challenge and bacterial clearance postinfection. Infect Immun. 2005; 73(9):5945-51. PubMed PMID: 16113315.
27. Joller N, Weber S S, Muller A J, Sporri R, Selchow P, Sander P, et al. Antibodies protect against intracellular bacteria by Fc receptor-mediated lysosomal targeting. Proc Natl Acad Sci USA. 2010; 107(47):20441-6. Epub 2010 Nov. 5. doi: 10.1073/pnas.1013827107. PubMed PMID: 21048081; PubMed Central PMCID: PMC2996673.
28. Jones S M, Ellis J F, Russell P, Griffin K F, Oyston P C. Passive protection against Burkholderia pseudomallei infection in mice by monoclonal antibodies against capsular polysaccharide, lipopolysaccharide or proteins. J Med Microbiol. 2002; 51(12):1055-62. PubMed PMID: 12466403.
29. Ngugi S A, Ventura V V, Qazi O, Harding S V, Kitto G B, Estes D M, et al. Lipopolysaccharide from Burkholderia thailandensis E264 provides protection in a murine model of melioidosis. Vaccine. 2010; 28(47):7551-5. Epub 2010 Sep. 15. doi: 10.1016/j.vaccine.2010.08.058. PubMed PMID: 20837078.
30. Zhang S, Feng S H, Li B, Kim H Y, Rodriguez J, Tsai S, et al. In Vitro and In Vivo studies of monoclonal antibodies with prominent bactericidal activity against Burkholderia pseudomallei and Burkholderia mallei. Clin Vaccine Immunol. 2011; 18(5):825-34. Epub 2011 Apr. 1. doi: 10.1128/CV1.00533-10. PubMed PMID: 21450976; PubMed Central PMCID: PMC3122519.
31. Chen Y S, Hsiao Y S, Lin H H, Liu Y, Chen Y L. CpG-modified plasmid DNA encoding flagellin improves immunogenicity and provides protection against Burkholderia pseudomallei infection in BALB/c mice. Infect Immun. 2006; 74(3):1699-705. PubMed PMID: 16495541.
32. Elvin S J, Healey G D, Westwood A, Knight S C, Eyles J E, Williamson E D. Protection against heterologous Burkholderia pseudomallei strains by dendritic cell immunization. Infect Immun. 2006; 74(3):1706-11. PubMed PMID: 16495542.
33. Cheng C, Pal S, Bettahi I, Oxford K L, Barry P A, de la Maza L M. Immunogenicity of a vaccine formulated with the Chlamydia trachomatis serovar F, native major outer membrane protein in a nonhuman primate model. Vaccine. 2011; 29(18):3456-64. Epub 2011 Mar. 8. doi: 10.1016/j.vaccine.2011.02.057. PubMed PMID: 21376796; PubMed Central PMCID: PMC3084512.
34. Malyala P, Singh M. Endotoxin limits in formulations for preclinical research. Journal of pharmaceutical sciences. 2008; 97(6):2041-4. Epub 2007 Sep. 12. doi: 10.1002/jps.21152. PubMed PMID: 17847072.
35. Nieves W, Heang J, Asakrah S, Honer zu Bentrup K, Roy C J, Morici L A. Immunospecific responses to bacterial elongation factor Tu during Burkholderia infection and immunization. PLoS One. 2010; 5(12):e14361. Epub 2010 Dec. 24. doi: 10.1371/journal.pone.0014361. PubMed PMID: 21179405; PubMed Central PMCID: PMC3003680.
36. Ho M, Schollaardt T, Smith M D, Perry M B, Brett P J, Chaowagul W, et al. Specificity and functional activity of anti-Burkholderia pseudomallei polysaccharide antibodies. Infect Immun. 1997; 65(9):3648-53. PubMed PMID: 9284132.
37. Collazo C M, Meierovics A I, De Pascalis R, Wu T H, Lyons C R, Elkins K L. T cells from lungs and livers of Francisella tularensis-immune mice control the growth of intracellular bacteria. Infect Immun. 2009; 77(5):2010-21. PubMed PMID: 19237526.
38. Darrah P A, Patel D T, De Luca P M, Lindsay R W, Davey D F, Flynn B J, et al. Multifunctional TH1 cells define a correlate of vaccine-mediated protection against Leishmania major. Nat Med. 2007; 13(7):843-50. PubMed PMID: 17558415.
39. Lindenstrom T, Agger E M, Korsholm K S, Darrah P A, Aagaard C, Seder R A, et al. Tuberculosis subunit vaccination provides long-term protective immunity characterized by multifunctional CD4 memory T cells. J Immunol. 2009; 182(12):8047-55. PubMed PMID: 19494330.
40. Roestenberg M, McCall M, Hopman J, Wiersma J, Luty A J, van Gernert G J, et al. Protection against a malaria challenge by sporozoite inoculation. N Engl J Med. 2009; 361(5):468-77. PubMed PMID: 19641203.
41. Fritz P E, Miller J G, Slayter M, Smith T J. Naturally occurring melioidosis in a colonized rhesus monkey (Macaca mulatta). Laboratory animals. 1986; 20(4):281-5. Epub 1986 Oct. 1. PubMed PMID: 3773432.

### Example 12

### Purification of OMVs

Described herein is an exemplary protocol that was used to extract B. thailandensis (Bt) or B. pseudomallei (Bp) naturally derived outer membrane vesicles (n-OMV) and eliminate other contaminants such as monomeric LPS (30 kD), whole cell bacteria and cellular fragments with the aid of the OptiPrep gradient buffer. Filter sterilization was used in this exemplary protocol to eliminate whole cell bacteria or large bacterial fragments. Ammonium sulfate precipitation was utilized to precipitate OMVs out of solution. See, for example, Moe et al., Infect. Immun., Vol. 70 No. 11 (2002); Bauman and Khuen, Microbes and Infection, 8 2400e2408 (2006); and Horstman and Khuen, J. Biol. Chem., Vol. 275 No. 17 (2000), the disclosures of all of which is hereby incorporated by reference.

This exemplary procedure protocol is formatted for a 500 mL culture supernatant which yields approximately 0.45 mg/mL n-OMV in a 300 ul-500 ul total volume. In a preferred embodiment, yields of OMVs was achieved from a total 1 L culture supernatant.

### Day 1:

5 mL culture of B. thailandensis (Bt) or B. pseudomallei (Bp) 1026b was grown overnight. One colony of Bt grown on a PIA plate (streaked from glycerol stock) was obtained to inoculate 5 mL LB broth. Grown overnight (0/N), 37° C., 233 rpm.

### Day 2:

Performed 1:100 dilution of the 0/N Bt culture into 495 mL of LB broth. Grew for 16 hours to late log phase-early stationary phase (OD ˜6.0), 37° C., 233 rpm.

### Day 3:

(1) Pelleted the whole Bt cells by centrifuging 6,000×g (6,300 rpm), 10 min, 4° C. (Use the SLA-1500 rotor for the Sorvall centrifuge).

(1)(a) Filled tubes to 80% avoiding overfill or spilled supernatant that may cause rotor to be unbalanced during spin.

(1)(b) Stored bacterial pellets at −80° C. for extraction of whole cell lysate (WCL), total membrane protein (TMP) and outer membrane protein (OMP) as described herein.

(1)(c) The supernatant contained the n-OMV. Repeated this step one more time to ensure no bacteria in the supernatant.

(2) Filtered the supernatant through a 0.22 um (sterile filtration) Millipore PES filter (Cat # SCGPU10RE) to remove any remaining bacteria or large bacterial fragments. Repeated this step one more time to ensure no bacteria in the supernatant.

(2)(a) To avoid clogging of the filters due to large amounts of bacteria left in supernatant, the 500 ml supernatant was filtered in two 250 mL filter containers.

(2)(b) Obtained 1 mL from step (b) and plated onto PIA agar. Incubated O/N, 37° C. where there was no growth. Allowed plate to stay in incubator up to 48 hrs (if needed) to further corroborate no bacterial growth as a quality control step (QC).

(3) Harvested the membrane vesicles in the filtered supernatant by slowly adding 1.5 M solid ammonium sulfate while slowly stirring ((NH2)4SO4 is from Fisher # A702-3). Incubated at 4° C. overnight (for a maximum of 48 hrs). The vesicles precipitated along with other contaminants (precipitate was a light brown color). Obtained 1 mL from step (c) and plated onto PIA agar. Incubated O/N, 37° C. There was no growth. Allowed plate to stay in incubator up to 48 hrs (if needed) to further corroborate no bacterial growth as a quality control step.

### Day 4:

(1) Ensured no growth in the PIA plate as bacterial growth is an indicator of bacterial contamination. Because there was no growth, proceeded with n-OMV extraction.

(2) The OMVs were pelleted by centrifugation at 11,000×g (8,500 rpm), 20 min, 4° C. using the SLA-1500 rotor for the Sorvall centrifuge. During this spin, prepared the Opti-Prep gradients (fresh).

(3) The precipitate (a light, thin smear) was resuspended in 2 ml, 10 mM HEPES/0.85% NaCl, pH 7.4 (HEPES-NaCl weight/volume). This was the crude vesicle preparation.

(4) Using the crude OMVs, 45% OptiPrep (Sigma) or other density gradient (ie. sucrose) was added in 10 mM HEPES/0.85% NaCl to approximately 4 mL total volume.

(5) To obtain debris-free OMV preparation, a density gradient was prepared as followed: layered on the bottom of a 26.3 mL centrifuge bottle (Beckman Coulter, 355618) the 4 mL of crude OMV from step (4) above; and very gently and slowly, layered over 4 mL 40%, 4 mL 35%, 6 mL 30%, 4 ml 25%, and 4 ml of 20% OptiPrep or Sucrose in HEPES-NaCl (w/v). The differences in the gradients reflected optimization in separating flagella and other soluble material from the vesicles.

(6) Centrifuged at 200,000×g (˜40,600 rpm), 1.5 hr, 4° C. using the Beckman rotor Type 50.2 Ti.

(7) 4 mL fractions were gently sequentially removed from the top and stored in 15 ml conical tubes at 4° C. (or continue to the next step).

### Analysis of Fraction Purity:

A portion of each fraction (˜1 mL from each fraction) was used to precipitate the OMVs with 20% tri-chloroacetic acid (TCA). The precipitated OMVs were used for western blotting in which Coomasie or silver staining gels with a 4-20% SDS-PAGE gel (Bio-Rad) was used. The most consistent fractions were pooled, and fractions containing unusual banding patterns indicative of contaminants were discarded.

### Purification of Vesicles:

Vesicles were recovered by pooling the peak fractions into a Beckman polycarbonate bottle as previously described herein. To make up the rest of the volume, 10 mM HEPES, pH6.8 was used. The n-OMVs were pelleted by centrifuging 200,000×g (40,600 rpm), 1.5 hr, 4° C. using the Beckman rotor 50.2 Ti as previously described herein.

(1) A small pellet containing pure OMVs resulted. The light brown gel-like pellet was resuspended in LPS-free water from Lonza (depending on pellet size). This was the final OMV preparation.

(2) Plates were spotted with at least 10% of the extracted OMVs (˜10 ul) on PIA agar and LB agar to ensure no bacterial contamination in the final OMV preparation.

(3) In an alternative embodiment, fractions were pooled in a 15 ml (max capacity) 100 kD Amicon tube to desalt the Opti-Prep out and to concentrate pooled OMVs. Centrifuge 2300×g, 25 min, 4° C. until all fractions were pooled. The final 2 spins were with 2 ml LPS-free water.

(4) The Bradford assay was performed to quantify final OMVs. Runs were pooled, desalted, and concentrated fractions (˜5 ug) were analyzed on an SDS-PAGE gel and stained with Coomasie (see attached TIF with SDS-PAGE analysis of OMV batches A-F. Each OMV batch was produced independently over a 1 year period to confirm reproducibility of the purification method).

### Storage:

Resuspended OMVs were aliquoted into 50-100 ul and stored at −20° C. In an alternative embodiment, OMVs were lyophilized for storage at 4° C. or at room temperature. The vesicles were checked for cleanliness (flagella and cell debris-free) by performing cryo transmission electron microscopy (TEM) as described, for example, in Nieves et al. (2010), the disclosure of which is incorporated herein by reference.

What is claimed is:

1. A composition comprising outer membrane vesicles of at least one Gram-negative bacteria.

2. The composition of claim 1, wherein said outer membrane vesicles further comprises lipopolysaccharide, and lacks adjuvant.

3. The composition of claim 1, wherein said outer membrane vesicles are derived from at least one Burkholderia spp.

4. The composition of claim 1 further comprising a pharmaceutically acceptable carrier.

5. The composition of claim 1 for protecting a mammal against infection caused by Gram-negative bacteria.

6. The composition of claim 5, wherein said Gram-negative bacteria is a Burkholderia species and the outer membrane vesicles are derived from the Burkholderia species.

7. A composition produced by the process of:
a. growing a culture of Gram-negative bacteria;
b. optionally subjecting the culture to oxidative or other environmental stress during said growth;
c. subjecting said culture to centrifugation, thereby obtaining a cell pellet and a supernatant fraction;
d. harvesting outer membrane vesicles from the supernatant fraction;
e. further purifying the outer membrane vesicles by gradient centrifugation; and
f. collecting said outer membrane vesicles.

8. The composition of claim 7, wherein said Gram-negative bacteria are Burkholderia.

9. An immunogenic composition comprising:
at least one purified outer membrane vesicle derived from at least one species of Burkholderia.

10. The immunogenic composition of claim 9, wherein the purified outer membrane vesicles further comprise lipopolysaccharide (LPS) and capsular polysaccharide (CPS).

11. The immunogenic composition of claim 9, wherein the purified outer membrane vesicles are derived from at least Burkholderia pseudomallei and/or Burkholderia mallei.

12. The immunogenic composition of claim 9, wherein the immunogenic composition is formulated as a vaccine.

13. The immunogenic composition of claim 9, wherein the immunogenic composition further comprises at least one adjuvant.

14. The immunogenic composition of claim 13, wherein the at least one adjuvant is selected from the group consisting of methylated CpG oligodeoxynucleotides (CpG ODN), aluminum hydroxide (alum), MPL-monophosphate lipid A, flagellin, cytokines, and toxins.

15. The immunogenic composition of claim 14, wherein the toxin is E. coli heat-labile enterotoxin and/or cholera toxin.

16. The immunogenic composition of claim 13, wherein the at least one adjuvant is an emulsions.

17. An immunogenic composition comprising purified outer membrane vesicles derived from at least one species of Burkholderia for protecting a subject against infection caused by at least one species of Burkholderia, wherein administration of the immunogenic composition provides protection against infection.

18. The immunogenic composition of claim 17, wherein the immunogenic composition is administered subcutaneously, intranasally, and/or intramuscularly.

19. The immunogenic composition of claim 17, wherein administration of the immunogenic composition produces protective humoral and cellular immunity to at least one species of Burkholderia.

20. The immunogenic composition of claim 19, wherein the protective humoral immunity in the subject comprises production of IgG and/or IgA specific to the administered outer membrane vesicles.

21. The immunogenic composition of claim 20, wherein production of IgG specific to the administered outer membrane vesicles increases by at least about 1-log when the immunogenic composition is subsequently administered.

22. The immunogenic composition of claim 20, wherein the IgG specific to the administered outer membrane vesicles comprises IgG1 and/or IgG2a.

23. The immunogenic composition of claim 19, wherein the protective cellular immunity in the subject comprises activation of memory T cells in response to the administered outer membrane vesicles.

24. The immunogenic composition of claim 23, wherein activation of memory T cells comprises production of interferon-gamma (IFN-γ) by Th1 memory cells.

25. The immunogenic composition of claim 19, wherein administration of the immunogenic composition provides protection when the subject is subsequently exposed to an aerosol challenge comprising at least one species of Burkholderia.

26. The immunogenic composition of claim 25, wherein the aerosol challenge comprises a lethal dose of the at least one species of Burkholderia.

27. The immunogenic composition of claim 19, wherein the subject is protected against infection caused by Burkholderia pseudomallei and/or Burkholderia mallei, and
wherein the immunogenic composition comprises purified outer membrane vesicles derived from at least Burkholderia pseudomallei and/or Burkholderia mallei.

28. An immunogenic composition according to claim 9 for inducing an immune response to at least one species of Burkholderia in a subject, wherein the immunogenic composition is administered to a subject in an amount effective to elicit production of antibodies specific to the at least one species of Burkholderia.

29. The immunogenic composition of claim 28, wherein the immunogenic composition is produced by:
a. growing a culture of Gram-negative bacteria;
b. subjecting said culture to centrifugation, thereby obtaining a cell pellet and a supernatant fraction;
c. harvesting outer membrane vesicles from the supernatant fraction;
d. purifying the outer membrane vesicles harvested from step (c) by gradient centrifugation; and
e. collecting the outer membrane vesicles purified from step (d).

30. The immunogenic composition of claim 29, wherein the gradient centrifugation of step (d) comprises high-speed centrifugation followed by density-gradient centrifugation.

31. An immunogenic composition comprising purified outer membrane vesicles derived from at least one species of Burkholderia for preventing respiratory infection in a subject wherein the respiratory infection is caused by at least one species of Burkholderia, wherein the immunogenic composition is administering to the subject, and wherein administration of the immunogenic composition prevents at least one symptom of said respiratory infection.

32. The immunogenic composition of claim 31, wherein the immunogenic composition is administered subcutaneously, intranasally, and/or intramuscularly.

33. The immunogenic composition of claim 31, wherein the respiratory infection is caused by Burkholderia pseudomallei and/or Burkholderia mallei, and
wherein the immunogenic composition comprises purified outer membrane vesicles derived from at least Burkholderia pseudomallei and/or Burkholderia mallei.

34. An immunogenic composition comprising purified outer membrane vesicles derived from at least one species of Burkholderia for preventing meliodosis in a subject wherein the meliodosis is caused by at least one species of Burkholderia, wherein the immunogenic composition is administered to the subject, and wherein administration of the immunogenic composition produces immunity to said at least one species of Burkholderia.

35. The immunogenic composition of claim 34, wherein the immunogenic composition is administered subcutaneously, intranasally, and/or intramuscularly.

36. The immunogenic composition of claim 34, wherein the immunity in the subject is protective humoral and cellular immunity.

37. The immunogenic composition of claim 34, wherein the protective humoral immunity in the subject comprises production of IgG and/or IgA specific to the administered outer membrane vesicles when the subject is exposed to at least one species of Burkholderia after administration of the immunogenic composition.

38. The immunogenic composition of claim 34, wherein the protective cellular immunity in the subject comprises activation of memory T cells in response to the administered outer membrane vesicles.

39. The immunogenic composition of claim 38, wherein activation of memory T cells comprises production of interferon-gamma (IFN-γ) by CD4+ and/or CD8+ T cells.

40. The immunogenic composition of claim 34, wherein administration of the immunogenic composition provides protection when the subject is subsequently exposed to an aerosol challenge comprising the at least one species of Burkholderia.

41. The immunogenic composition of claim 34, wherein the meliodosis is pneumonic meliodosis and/or septicemic meliodosis.

42. The immunogenic composition of claim 34, wherein the meliodosis is caused by Burkholderia pseudomallei, and
wherein the immunogenic composition comprises purified outer membrane vesicles derived from at least Burkholderia pseudomallei.

43. The immunogenic composition of claim 34, wherein the immunogenic composition further comprises at least one adjuvant.

44. The immunogenic composition of claim 43, wherein the at least one adjuvant is selected from the group consisting of methylated CpG oligodeoxynucleotides (CpG ODN), aluminum hydroxide (alum), MPL-monophosphate lipid A, flagellin, cytokines, and toxins.

45. The immunogenic composition of claim 44, wherein the toxin is E. coli heat-labile enterotoxin and/or cholera toxin.

46. The immunogenic composition of claim 43, wherein the at least one adjuvant is an emulsions.

47. An immunogenic composition comprising purified outer membrane vesicles derived from at least one species of Burkholderia cepacia complex for preventing respiratory infection in a subject wherein the respiratory infection is caused by at least one species of Burkholderia cepacia complex, wherein the immunogenic composition is administered to the subject, wherein administration of the immunogenic composition produces immunity to said at least one species of Burkholderia cepacia complex, and wherein administration of the immunogenic composition prevents at least one symptom of said respiratory infection.

48. The immunogenic composition of claim 47, wherein the immunity in the subject is protective humoral and/or cellular immunity.

49. The immunogenic composition of claim 47, wherein the respiratory infection is rapidly fatal pulmonary infection.

50. The immunogenic composition of claim 47, wherein the subject is afflicted with cystic fibrosis.

51. The immunogenic composition of claim 47, wherein the respiratory infection is caused by Burkholderia cenocepacia and/or Burkholderia multivorans, and
wherein the immunogenic composition comprises purified outer membrane vesicles derived from Burkholderia cenocepacia and/or Burkholderia multivorans.
